(19) United States
(12) Patent Application Publication
(10) Pub. No.: US 2025/0195448 A1
(43) Pub. Date: Jun. 19, 2025

(54) CELL TYPE SELECTIVE DELIVERY OF EXOSOME CARGO USING NOTCH LIGAND-RECEPTOR SYSTEM

(71) Applicant: Northwestern University, Evanston, IL (US)

(72) Inventors: Jeffrey Nicholas Savas, Evanston, IL (US); Yi-Zhi Wang, Evanston, IL (US)

(21) Appl. No.: 18/985,235

(22) Filed: Dec. 18, 2024

Related U.S. Application Data
(60) Provisional application No. 63/611,324, filed on Dec. 18, 2023.

(51) Int. Cl. A61K 9/51; A61K 31/711; C07K 14/705

(52) U.S. Cl. CPC A61K 9/5176; A61K 31/711; C07K 14/705

(57) ABSTRACT

Provided herein are engineered exosomes and uses thereof for cell-type selective delivery of cargo. In particular, provided herein are engineered exosomes expressing a Notch ligand binding domain and uses thereof for targeted delivery of therapeutic cargo to cells expressing Notch ligand, such as neurons.

### PRIORITY STATEMENT

This application claims priority to U.S. Provisional Application No. 63/611,324, filed Dec. 18, 2023, the entire contents of which are incorporated herein by reference for all purposes.

### STATEMENT REGARDING FEDERALLY SPONSORED RESEARCH

This invention was made with government support under AG061787 awarded by the National Institutes of Health. The government has certain rights in the invention.

### SEQUENCE LISTING

The text of the computer readable sequence listing filed herewith, titled “NWEST_42603_202_SequenceListing.xml”, created Dec. 17, 2024, having a file size of 42,682 bytes, is hereby incorporated by reference in its entirety.

### FIELD

Provided herein are engineered exosomes and uses thereof for cell-type selective delivery of cargo. In particular, provided herein are engineered exosomes expressing a Notch ligand binding domain and uses thereof for targeted delivery of therapeutic cargo to cells expressing Notch ligand, such as neurons.

### BACKGROUND

Neurons communicate with each other by selectively sending and receiving chemical and electrical signals. Recently, extracellular vesicles (EVs) have emerged as additional potential mediators of intercellular communication in the central nervous system (CNS). However, methods to selectively target EVs to a desired cell type are lacking, as an EV receptor system in the CNS has not been identified. There is a need for safe, effective, and targeted delivery of therapies such as proteins, small molecules, nucleic acids, RNA, and DNA to treat various disorders including neurological disorders (e.g. lysosomal storage disorders) and cancers of the central nervous system. In particular, diseases where enzymes that degrade proteins, lipids, or carbohydrates are dysfunctional don't have sufficient therapies, and are in need of cell-type specific targeting of enzyme replacement.

Notch is a highly-conserved, single-pass transmembrane protein expressed in multiple cell types in most animals. Notch is involved in organismal development and cell differentiation. However, the Notch ligand and receptor system has never previously been utilized as a mechanism by which exosomes can be engineered to target delivery of cargo to specific cell types, such as neurons.

### SUMMARY

In some aspects, provided herein are engineered exosomes comprising a Notch ligand binding domain and a therapeutic cargo. The engineered exosomes provided herein are internalized by cells expressing a Notch receptor ligand, such as neurons, and thus find use in methods of treating neurological disease in a cell-type-specific manner. For example, the engineered exosomes herein may be used in enzyme replacement therapy (ERT) for neurological disorders such as lysosomal storage disorders.

In some aspects, provided herein is an engineered exosome comprising a Notch ligand binding domain, wherein the Notch ligand binding domain is expressed on an extracellular surface of the engineered exosome; and a therapeutic cargo. In some embodiments, binding of a Notch ligand to the Notch ligand binding domain induces internalization of the engineered exosome into cells expressing the Notch ligand. In some embodiments, the cell is a neuron.

In some embodiments, the Notch ligand binding domain comprises a polypeptide having at least 80% sequence identity with SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24. In some embodiments, the Notch ligand binding domain comprises SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24.

In some embodiments, the engineered exosome comprises at least a portion of an extracellular domain of a Notch receptor, wherein the extracellular domain includes the Notch ligand binding domain. In some embodiments, the Notch receptor is human Notch1, human Notch2, human Notch3, or human Notch4. In some embodiments, the at least a portion of the extracellular domain of the Notch receptor comprises a polypeptide having at least 80% sequence identity with SEQ ID NO: 5, SEQ ID NO: 6, SEQ ID NO: 7, or SEQ ID NO: 8. In some embodiments, the engineered exosome comprises SEQ ID NO: 5, SEQ ID NO: 6, SEQ ID NO: 7, or SEQ ID NO: 8. In some embodiments, the engineered exosome comprises the extracellular domain and the transmembrane domain of SEQ ID NO: 1, SEQ ID NO: 2, SEQ ID NO: 3, or SEQ ID NO: 4.

In some embodiments, the engineered exosome natively expresses a wildtype Notch receptor, such that the Notch ligand binding domain is natively expressed on the extracellular surface of the engineered exosome. For example, in some embodiments the exosome natively expresses a polypeptide having at least 80% identity with SEQ ID NO: 1, SEQ ID NO: 2, SEQ ID NO: 3, or SEQ ID NO: 4. For example, in some embodiments the exosome natively expresses the Notch receptor of SEQ ID NO: 1, SEQ ID NO: 2, SEQ ID NO: 3, or SEQ ID NO: 4.

In some embodiments, the therapeutic cargo is an agent for the treatment of a neurological disease or condition, a central nervous system cancer, or a central nervous system injury. Any suitable therapeutic cargo may be used, including a nucleic acid, a peptide, a protein, an antibody, an aptamer, or a small molecule. For example, in some embodiments therapeutic cargo is an mRNA.

In some aspects, provided herein is a method comprising providing to a subject an engineered exosome described herein. In some embodiments, the subject has or is suspected of having a neurological disease or condition, a central nervous system cancer, or a central nervous system injury.

In some aspects, provided herein is a method of treating a neurological disease or condition, a central nervous cancer, or a central nervous system injury in a subject, comprising providing to the subject an engineered exosome described herein. In some embodiments, the therapeutic cargo is selectively delivered to neurons in the subject after internalization of the engineered exosome into neurons expressing a Notch ligand.

### BRIEF DESCRIPTION OF THE DRAWINGS

FIGS. 1A-1F show synaptic NMDAR activation triggers secretion of EVs containing Notch1 and Notch2 proteins. (FIG. 1A) Experimental scheme depicting EV purification workflow and cargo analyses. (FIG. 1B) Representative images of EV particles present in SEC fraction 2 (F2) visualized by negative staining EM. Scale bar, 500 nm, inset scale bar, 50 nm. (FIG. 1C) The diameter range of EV-like particles. n=299 particles from two biological replicates. (FIG. 1D) The EV markers Alix, Tsg101, and CD81 but not GM130 are selectively detected in SEC F2 isolated from glycine stimulated neuronal cultures. (FIG. 1E) Proteomic analysis reveals that Notch1, and Notch2 are highly abundant in SEC F2 along with other known EV protein markers. Mean±S.E.M. from 3 biological replicates. Insert: Notch 1 and 2 peptides identified by MS/MS. (FIG. 1F) WB validation of Notch1_{ICD} and Notch2_{ICD} in SEC F2 (top). Silver-stained gel indicating the total amount of protein loaded across the 10 fractions (bottom).

FIGS. 2A-2I show notch receptor-ligand interactions mediate EV internalization selectively by neurons. (FIG. 2A) Proteinase K (PK) treatment reduces the apparent Notch1 and Notch2 molecular weight by ˜10 kDa, consistent with removal of the extracellular portion of Notch_{ICD}. No intact CD81 was detected in the PK treated EVs presumably due to multiple cleavage events, while Sdcbp was unaffected by PK treatment. (FIG. 2B) Schematic illustrating the arrangement of Notch1, Notch2, CD81 and Sdcbp in EVs. (FIG. 2C) Representative ICC images showing Notch ligands Jag 1, Jag2, Dll1 and Dll4 co-localize with internalized neuronal EVs, labeled with CFSE. Top, neuronal soma, scale bar, 10 μm, bottom, dendrites, scale bar, 5 μm. (FIG. 2D) Quantification of the percentage of CFSE-labelled EVs colocalized with the indicated Notch ligands in neuronal soma. One-tailed Student's t-test, n=12-15 from two cultures. NS, not significant. *p-value (Jag1 vs Jag2)=0.0204, **p-value (Jag1 vs Dll1)=0.0082, **p-value (Jag1 vs Dll4)=0.0051, p-value (Jag2 vs Dll1)=0.2947, p-value (Jag2 vs Dll4)=0.2476, p-value (Dll1 vs Dll4)=0.4396. (FIG. 2E) Left, CFSE labeled EVs that are internalized by primary cultured rat hippocampal neurons remain generally intact and punctate. Right, neurons fail to internalize PK-treated EVs. Scale bar, 10 μm. (FIG. 2F) Following a 60-minute incubation of primary cultured rat hippocampal neurons with EVs, there is a notable increase in the levels of activated-Notch1, -Notch2, and Hes1 proteins. This effect is not observed with EVs treated with Delta^{MAX} or with Delta^{MAX} treatment alone. (FIG. G, FIG. 2H, FIG. 2I), Quantifications of (FIG. 2F). One-tailed Student's t-test, n=4 biological. (FIG. 2G) ***p-value (Veh vs EV)=0.0002, ***p-value (EV vs Delta^{MAX}-treated EV)=0.0004, NS p-value (Delta^{MAX}-treated EV vs Delta^{MAX})=0.4453. (FIG. 2H) **p-value (Veh vs EV)=0.0043, **p-value (EV vs Delta^{MAX}-treated EV)=0.0042, NS p-value (Delta^{MAX}-treated EV vs Delta^{MAX})=0.4927. (FIG. 2I) ***p-value (Veh vs EV)=0.0004, **p-value (EV vs Delta^{MAX}-treated EV)=0.0011, NS p-value (Delta^{MAX}-treated EV vs Delta^{MAX}) 0.2677.

FIGS. 3A-3E show activation of synaptic NMDARs drives Notch target gene expression. (FIG. 3A) Top, experimental scheme showing the analysis time points for bulk neuron RNA analysis. Bottom, biological replicates cluster by analysis time point in multidimensional scaling plots. (FIG. 3B) Heatmap showing a panel of Notch target genes that are activated by Mg²⁺-free glycine treatment. (FIG. 3C) Volcano plot depicting comparison of mRNA levels in Veh-treated neurons (5 mins after treatment) and Mg²⁺-free glycine-treated neurons (60 mins after treatment). One-tailed Student's t-test. (FIG. 3D) Representative WB blot showing Mg²⁺-free glycine treatment also elevated the levels of activated-Notch1, -Notch2 and Hes1in primary cultured rat hippocampal neurons (90 mins after treatment), which can be inhibited by either D-2-amino-5-phosphonovalerate (APV) or dynasore. (FIG. 3E) Quantification of (FIG. 3D). n=4 biological replicates. One-tailed Student's t-test. Hes1: **p-value (Veh vs Gly)=0.0065, *p-value (Gly vs +APV)=0.0102, **p-value (+DMSO vs +Dynasore)=0.0076. Activated-Notch1: **p-value (Veh vs Gly)=0.0003, **p-value (Gly vs +APV)=0.0008, *p-value (+DMSO vs +Dynasore)=0.0102. Activated-Notch2: ***p-value (Veh vs Gly)=7.466E-05, ***p-value (Gly vs +APV)=5.215E-05, **p-value (+DMSO vs +Dynasore)=0.0015.

FIGS. 4A-4L show activation of synaptic NMDARs is insufficient to trigger the Notch EV signaling pathway in Alix^{-/-} neurons. (FIG. 4A) Top, representative fluorescent detection of Mg²⁺-free glycine-induced EVs from neurons with indicated genotypes. Scale bar, m. Bottom, quantification of Mg²⁺-free glycine-induced neuronal EVs immunocaptured from indicated neuron genotypes. n=3 biological replicates (BR). Each chip contains three technical replicates. One-tailed Student's t-test. ***p-value (Alix^{+/+} vs Alix^{+/-})=7.821-E07, ***p-value (Alix^{+/+} vs Alix^{-/-})=3.143-E15, **p-value (Alix^{+/-} vs Alix^{-/-})=0.0060. (FIG. 4B) Left, WB analysis showing Mg²⁺-free glycine stimulation failed to induce EV release in Alix^{-/-} hippocampal neurons. Right, silver-stained gel indicating the total amount of protein recovered across the 10 size exclusion fractions. (FIG. 4C) WB analysis showing Mg²⁺-free glycine stimulation failed to upregulate the level of activated-Notch1, -Notch2 and Hes1in Alix^{-/-} hippocampal neurons. (FIG. 4D) Quantification of (FIG. 4C). n=3 biological replicates. One-tailed Student's t-test. NS, not significant. Activated-Notch1: p-value=0.3598. Activated-Notch2: p-value=0.2162, Hes1: p-value=0.2737. (FIG. 4E) WB analysis showing Mg²⁺-free glycine stimulation elevated the levels of activated-Notch1, -Notch2 and Hes1in Alix^{+/+} but not Alix^{-/-} hippocampal neurons from littermates. (FIG. 4F) Quantification of (FIG. 4E). n=4 cultures each genotype. One-tailed Student's t-test. Activated-Notch1: *p-value=0.0128, Activated-Notch2: ***p-value=0.0007, Hes1, ***p-value=0.0004. (FIG. 4G) Mg²⁺-free glycine stimulation leads to Alix phosphorylation, which can be inhibited by PKA inhibitor H89. p-S/T, phosphorylated serine or threonine. Stau, staurosporine. (FIG. 4H) Quantification of (FIG. 4G). n=4 biological replicates. One-tailed Student's t-test. NS, not significant. ***p-value (Veh+DMSO vs Gly+DMSO)=1.050E-07, p-value (Veh+DMSO vs Gly+H89)=0.1624, ***p-value (Gly+DMSO vs Gly+H89)=2.443E-07, p-value (Gly+DMSO vs Gly+Stau)=0.3850. (FIG. 4I) Representative MS² spectra indicating Alix phosphorylation at S717 from rat hippocampal neuron whole cell extracts treated with Mg²⁺-free glycine. Assigned fragment ions are indicated in b (blue), y (red) and those containing phosphorylated Serine 717 are labeled. (FIG. 4J) AlphaFold 2-predicted 3D protein structures of rat Alix, indicating the position of S717. (FIG. 4K) Overexpression of mCherry-Alix or mCherry-Alix-S717D rescues Mg²⁺-free glycine-induced EV release from Alix^{-/-} neurons. Scale bar, 10 μm. (FIG. 4L) Quantification of (FIG. 4K). n=4-7 biological replicates. Each chip contains three technical replicates. One-tailed Student's t-test. NS, not significant. ***p-value (mCherry vs mCherry-Alix)=4.249E-06, p-value (mCherry vs mCherry-Alix-S717A)=0.2110, ***p-value (mCherry vs mCherry-Alix-S717D)=6.004E-13, ***p-value (mCherry-Alix vs mCherry-Alix-S717A)=5.468E-10, p-value (mCherry-Alix vs mCherry-Alix-S717D)=0.1312.

FIGS. 5A-5C show notch pathway in hippocampus of juvenile Alix^{-/-} mouse. (FIG. 5A) WB analyses showing that, at P0 and P4, the levels of activated-Notch1, -Notch2, Notch1_{ICD}, Notch2_{ICD} and Hes1 are similar in Alix^{+/+} and Alix^{-/-} hippocampi. However, at P14, the levels of activated-Notch1, -Notch2 and Hes1 are significantly reduced in Alix^{-/-} hippocampus. (FIG. 5B) Quantification of (FIG. 5A). n=3 mice for each group. One-tailed Student's t-test, NS, not significant. P0: Activated Notch1 p-value=0.3752, Activated Notch2 p-value=0.3865, Notch1_{ICD} p-value=0.2047, Notch2_{ICD} p-value=0.3590, Hes1 p-value=0.1232. P4: Activated Notch1 p-value=0.2149, Activated Notch2 p-value=0.2743, Notch1_{ICD}p-value=0.3837, Notch2_{ICD} p-value=0.4283, Hes1 p-value=0.0836. P14: Activated Notch1 *p-value=0.0324, Activated Notch2 *p-value=0.0265, Notch1_{ICD} p-value=0.1029, Notch2_{ICD} p-value=0.4227, Hes1 **p-value=0.0042. (FIG. 5C) At P0 and P4, the expression patterns of Notch1_{ICD} and Notch2_{ICD} are similar in Alix^{+/+} and Alix^{-/-} hippocampal CA1 regions. At P14, the amount of nuclear-localized Notch1_{ICD} and Notch2_{ICD} are much less in Alix^{-/-} hippocampal CA1 regions, compared to Alix^{+/+} hippocampus. Scale bar, 10 μm.

FIGS. 6A-6D show conditional deletion of Alix in adult mouse hippocampus reduces Notch signaling pathway activation. (FIG. 6A) The levels of activated-Notch1, -Notch2 and Hes1 are significantly reduced in hippocampus from ˜2-month-old Camk2a-cre::Alix^{fl/fl} mice, compared to Alix^{fl/fl} mice. M=mouse. (FIG. 6B) Quantification of (FIG. 6A), n=4 mouse per group. One-tailed Student's t-test, NS, not significant. Activated Notch1: *p-value=0.0143, Activated Notch2: **p-value=0.0075, Notch1_{ICD}: p-value=0.0942, Notch2_{ICD}: p-value=0.4709, Hes1: *p-value=0.0218, Alix: ***p-value=5.346E-05. (FIG. 6C) Lack of Alix in adult hippocampus led to alteration of nuclear-localized Notch1_{ICD} and Notch2_{ICD}. Scale bar, 10 μm. (FIG. 6D) Quantification of (FIG. 6C), n=5 mouse per group. One-tailed Student's t-test, NS, not significant. Nucleic Notch1_{ICD}/cytosolic Notch1_{ICD}: CA1, **p-value=0.0042, CA3, **p-value=0.0037, DG, ***p-value=0.0002. Nucleic Notch2_{ICD}/cytosolic Notch2_{ICD}: CA1, **p-value=0.0026, CA3, ***p-value=0.0009, DG, p-value=0.3942.

FIGS. 7A-7D show conditional deletion of Alix in adult mouse hippocampus disrupts glutamatergic synapse protein expression. (FIG. 7A) Experimental design to examine subregion-specific proteomic differences in the hippocampus between Camk2a-cre:: Alix^{fl/fl} and Alix^{fl/fl} mice. (FIG. 7B) The t-SNE (t-distributed stochastic neighbor embedding) plot illustrate dissimilar protein expression in of all samples. BR, biological replicate. (FIG. 7C) Volcano plots depicting comparison of Camk2a-cre:: Alix^{fl/fl} and Alix^{fl/fl} hippocampal subregion specific proteomes. n=4 mice per experimental group. Pie charts summarizing proteins differently expressed by WT and cKO mice in each hippocampal region. Two-way ANOVA. (FIG. 7D) GO::CC gene annotation analysis suggests lack of Alix expression in adult hippocampus mainly affects glutamatergic synapse in CA1, CA3 an DG. List of the top 10 most significantly enriched terms for both significantly down- and up-regulated proteins.

FIGS. 8F-8K show glycine treatment increases excitatory synapse activity in primary cultured hippocampal neurons, related FIG. 1. (FIG. 8A) Schematic cartoon of NMDAR-dependent mechanism underlying enhancement of excitatory synapse activity by Mg²⁺-free glycine stimulation. (FIG. 8B) Mg²⁺-free glycine treatment induces phosphorylation of Ser295 (pS295) in PSD95 by activation of NMDAR. (FIG. 8C) Quantification of (FIG. 8B). n=5 biological replicates. One-tailed Student's t-test, NS, not significant. ***p-value (Veh vs Gly)=0.0002, p-value (Veh vs Gly+APV)=0.1816. (FIG. 8D) Representative voltage clamp recordings of mEPSCs during baseline and 25-30 mins after glycine treatment in absence of (top) or presence of APV (50 μM) (bottom). (FIG. 8E) Time course of glycine-induced potentiation of mEPSCs. (FIG. 8F) Summary of the effect of glycine on mEPSC amplitude on individual recordings. (FIG. 8G) Summary of the effect of glycine on mEPSC amplitude on individual recordings in the presence of D-APV. Individual recordings were characterized as “no potentiation” if the amplitude at 25-30 mins after glycine was not increased by more than 10% from the mean of the 5-minute baseline mEPSC amplitude. (FIG. 8H) Top, representative images showing Mg²⁺-free glycine treatment enlarged dendritic spines. Bottom, quantification of spine enlargement. Mean □ S.E, n=26 dendritic segments from four biological replicates, one-tailed Student's t-test, NS, not significant. p-value (−20 min vs 0 min)=0.3666, p-value (7 min vs 0 min)=0.3114, ***p-value (10 min vs 0 min)=4.656E-08, ***p-value (20 min vs 0 min)=7.252E-05, ***p-value (40 min vs 0 min)=1.622E-09. Scare bar, 1 μm. (FIG. 8I) Glycine treatment does not cause plasma membrane rapture. n=3 biological replicates. One-tailed Student's t-test, NS, not significant. p-value (medium vs Veh)=0.4870, p-value (Veh vs Gly)=0.4686. (FIG. 8J) Representative negative staining EM images of SEC F3 of Mg²⁺-free glycine-treated neuronal cultures. Scale bar=500 nm. (FIG. 8K) Representative negative staining EM images of SEC F2 and F3 of vehicle-treated neuronal cultures. Scale bar=500 nm.

FIGS. 9A-9H show notch ligands are predominantly expressed in neurons and dendrites are the major sites of synaptic NMDAR activation-induced neuronal EVs release, related FIG. 2. (FIG. 9A) Representative ICC images showing that in neuron-glia mixed cultures, most Notch ligands are expressed in neurons. Scale bar, 10 μm. (FIG. 9B) Most Jag1, Jag2, Dll1 and Dll4 are co-stained with excitatory presynaptic terminal marker, VGluT1. A part of Jag1 only co-stained with excitatory postsynaptic marker, homer1. Arrowhead, Notch ligands co-stained with both VGluT1 and homer1. Arrow, Jag1 only co-stained with homer1. Scale bar, 1 μm. (FIG. 9C) Quantification of (FIG. 9B). n=15-18 dendritic segments from two cultures. One-tailed Student's t-test, NS, not significant. Co-localization with Homer1: ***p-value (Jag1 vs Jag2)=6.802E-08, ***p-value (Jag1 vs Dll1)=3.118E-10, ***p-value (Jag1 vs Dll4)=1.955E-07, p-value (Jag2 vs Dll1)=0.4200, *p-value (Jag2 vs Dll4)=0.0452, *p-value (Dll1 vs Dll4)=0.0308. Co-localization with VGluT1: ***p-value (Jag1 vs Jag2)=8.433E-12, ***p-value (Jag1 vs Dll1)=2.860E-13, ***p-value (Jag1 vs Dll4)=1.814E-13, p-value (Jag2 vs Dll1)=0.3067, p-value (Jag2 vs Dll4)=0.2326, p-value (Dll1 vs Dll4)=0.3997. (FIG. 9D) Notch1 and 2 proteins are colocalized with excitatory postsynaptic protein marker PSD95. Arrow head, Notch proteins only co-stained with PSD95 but not VGluT1. Scale bar, 1 μm. (FIG. 9E) Subcellular fractionation analysis of mouse cortex reveals the presence of Alix, Tsg101, Sdcbp, Notch1_{ICD}, Notch2_{ICD}, Dll1, and Dll4 in the postsynaptic density (PSD) fraction, while they are absent in the presynaptic (Pre) fraction. Jag1 and Jag2 are detected in both the Pre and PSD fractions. S1, supernatant 1, P1, pellet 1, S2 supernatant 2, P2, pellet 2, SS, synaptosome. (FIG. 9F) In neurons overexpressing mCherry-Alix and GFP, several mCherry-positive protrusions (indicated by *) showed at the tips of dendritic spines after Mg²⁺-free glycine stimulation. Scale bars, 1 μm. (FIG. 9G) Mg²⁺-free glycine stimulation failed to induce mCherry-positive protrusion in neurons overexpressing mCherry and GFP. Scale bars, 1 μm. (FIG. 9H) Mg²⁺-free glycine stimulation cannot induce mCherry-positive protrusion out of soma of neurons overexpressing mCherry-Alix and GFP. Scale bar, 10 μm.

FIGS. 10A-10J show neurons preferentially internalize neuronal EVs induced by synaptic NMDAR activation, related FIG. 2 (FIG. 10A) The lipid dye CM-Dil labelled neuronal EVs are internalized by neurons and kept the membrane integrity. Top, neuronal soma, Scale bar, 10 μm; bottom, dendrite, Scale bar, 1 μm. (FIG. 10B) Incubation protein dye CFSE labelled EVs in glia-neuron mixed cultures, most EVs are specifically internalized by neurons. Scale bar, 100 μm. (FIG. 10C) Quantification of (FIG. 10B). n=16 field from two biological replicates. (FIG. 10D) Diagram illustrating the procedure for treating neurons with Notch1-myc EVs. The 6× myc tags was put into the C-terminal of mouse Notch1₁₋₂₁₈₄. (FIG. 10E) Top: the majority of Notch1-myc EVs were taken up by neurons, as indicated by the presence of MAP2-positive cells. Arrowheads mark the instances of myc and DAPI colocalization, suggesting the translocation of Notch1_{ICD} into the nucleus. Scale bar, 100 μm. Bottom: The colocalization of myc and DAPI in a Notch1-myc EVs-treated neuron. Scale bar, 10 μm. (FIG. 10F) Incubation PK-treated EVs in glia-neuron mixed cultures, nearly no EV integrity uptake was observed. The week smeared CFSE signals in somas may be due to the fusion of EV membrane to neuronal plasma membrane and release CFSE-labelled protein cargo. Scale bar, 100 μm. (FIG. 10G) Sixty-minute incubation of primary cultured rat hippocampal neurons with EVs, but not PK-treated EVs, causes elevated levels of activated-Notch1, -Notch2 and Hes1 proteins. (FIG. 10H, FIG. 10I, FIG. 10J), Quantifications of (FIG. 10G). One-tailed Student's t-test, n=3 biological. (FIG. 10H) **p-value (Veh vs EV)=0.0021, **p-value (EV vs PK-treated EV)=0.0064. (FIG. 10I) **p-value (Veh vs EV)=0.0054, *p-value (EV vs PK-treated EV)=0.0124. (FIG. 10J) ***p-value (Veh vs EV)=0.0006, **p-value (EV vs PK-treated EV)=0.0015.

FIGS. 11A-11G show deletion of Alix has a minor effect on the subcellular localization of Notch1 protein, related FIGS. 3 & 4. (FIG. 11A) Exposure primary cultured rat hippocampal neurons to EVs for 60 minutes results in an increase in dendritic spine density. Scale bar, 4 μm. (FIG. 11B) Quantification of (FIG. 11A), The analysis was conducted on 26 dendrite segments from neurons treated with PBS and 29 segments from neurons treated with EVs. Neurons were obtained from three separate cultures. Spine density, ***p-value=4.9134E-5, spine length, NS p-value=0.4221, spine head diameter, NS p-value=0.1081. (FIG. 11C) WB analysis of Alix protein level in Alix^{+/+}, Alix^{+/-} and Alix^{-/-} hippocampal neurons. (FIG. 11D) The amount of CD81 positive particles captured by normal hamster or rat IgG was used to estimate the amount of sample loading in each ExoView chips. Scale bar, 10 μm. (FIG. 11E) Quantification of (FIG. 11D), n=3 biological replicates. Each chip contains three technical replicates. One-tailed Student's t-test, NS, not significant. Particles captured by rat IgG trap: p-value (Alix^{+/+} vs Alix^{+/-})=0.1086, p-value (Alix^{+/+} vs Alix^{-/-})=0.3153, p-value (Alix^{+/-} vs Alix^{-/-})=0.1682. Particles captured by hamster IgG trap: *p-value (Alix^{+/+} vs Alix^{+/-})=0.0106, p-value (Alix^{+/+} vs Alix^{-/-})=0.2113, p-value (Alix^{+/-} vs Alix^{-/-})=0.4614. (FIG. 11F) Representative ICC images showing the colocalization of Notch1 with Tsg101 or LBPA in primary cultured Alix^{+/+} and Alix^{-/-} hippocampal neurons. Arrow head pointed the colocalized puncta. Scale bar, 1 μm. (FIG. 11G) Quantification of (FIG. 11F), n=15-34 dendritic segments from 2 biological replicates. One-tailed Student's t-test. Colocalization of Notch1 with Tsg101: *p-value=0.0237, Colocalization of Notch1 with LBPA: ***p-value=0.0003.

FIG. 12A-12G Mg²⁺-free glycine stimulation leads to Alix phosphorylation, related FIG. 4. (FIG. 12A) Lambda phosphatase (λ-PP) treatment greatly reduced the level of p-S/T in the immunoprecipitants of anti-Alix from Mg²⁺-free glycine-treated neurons. (FIG. 12B) NMDAR inhibitor APV significantly reduced the phosphorylation level of Alix in Mg²⁺-free glycine-treated hippocampal neurons. (FIG. 12C) Quantification of (FIG. 12B). n=5 biological replicates. One-tailed Student's t-test. ***p-value (Veh+DMSO vs Gly+DMSO)=0.0004, **p-value (Gly+DMSO vs Gly+APV)=0.0046. (FIG. 12D) Camk2 inhibitors AIP and KN93 failed to block Mg²⁺-free glycine-induced Alix phosphorylation. (FIG. 12E) APV and H89 blocked Mg²⁺-free glycine-induced EV release. (FIG. 12F) The amount of CD81 positive particles captured by normal hamster or rat IgG was used to estimate the amount of sample loading in each ExoView chips. (FIG. 12G) Quantification of (FIG. 12F). n=4-7 biological replicates. Each chip contains three technical replicates. One-tailed Student's t-test, NS, not significant. Particles captured by rat IgG trap: *p-value (mCherry vs mCherry-Alix)=0.0489, **p-value (mCherry vs mCherry-Alix-S717A)=0.0059, **p-value (mCherry vs mCherry-Alix-S717D)=0.0032, p-value (mCherry-Alix vs mCherry-Alix-S717A)=0.2392, ***p-value (mCherry-Alix vs mCherry-Alix-S717D)=0.0003. Particles captured by hamster IgG trap: p-value (mCherry vs mCherry-Alix)=0.1436, ***p-value (mCherry vs mCherry-Alix-S717A)=0.0010, **p-value (mCherry vs mCherry-Alix-S717D)=0.0021, ***p-value (mCherry-Alix vs mCherry-Alix-S717A)=0.0004, ***p-value (mCherry-Alix vs mCherry-Alix-S717D)=5.305E-06. Scale bar, 10 μm.

FIGS. 13A-13B show Alix global KO mice have reduced nucleic Notch1_{ICD} and Notch2_{ICD} levels in the hippocampus of P14 mice, related FIG. 5. (FIG. 13A) At P0 and P4, the expression patterns of Notch1_{ICD} are similar in Alix^{+/+} and Alix^{-/-} hippocampal CA3 and DG regions. At P14, the amount of nuclear-localized Notch1_{ICD} is much less in Alix^{-/-} hippocampal regions, compared to Alix^{+/+} hippocampus. Scale bar, 10 μm. (FIG. 13B) At P0 and P4, the expression patterns of Notch2_{ICD} are similar in Alix^{+/+} and Alix^{-/-} hippocampal CA3 and DG regions. At P14, the amount of nuclear-localized Notch2_{ICD} are reduced in Alix^{-/-} hippocampal CA3 regions. However, the expression patterns of Notch2_{ICD} are similar in Alix^{+/+} and Alix^{-/-} DG. Scale bar, 10 μm.

FIGS. 14A-14B show hippocampus-region specific synaptic protein alteration caused by conditional Alix deletion in adult mice, related FIG. 7. (FIG. 14A) The Venn diagrams illustrate the differences in proteomic changes in the CA1, CA3, and DG regions, resulting from the conditional deletion of Alix. (FIG. 14B) The heatmap summarizes the hippocampal region-specific synaptic protein alterations in Alix cKO mice in comparison to WT mice. Each data point represents the mean of TMT-MS measurements obtained from four mice.

FIGS. 15A-15E show characterization of RNA cargo in neuronal extracellular vesicles (EV) induced by three chemical synaptic stimulations (glycine, forskolin, and DHPG). (FIG. 15A) Experimental scheme depicting EV purification workflow and RNA sequencing. (FIG. 15B) Pie charts summarizing RNA sequencing results for glycine-induced EVs (Gly-EVs), forskolin-induced EVs (Fos-EVs), DHPG-induced EVs (DHPG-EVs), and the negative control (Neg-CTRL). (FIG. 15C) Upset plot illustrating the intersections of RNAs identified in Gly-EVs, Fos-EVs, DHPG-EVs, and Neg-CTRL. (FIG. 15D) Volcano plot comparing RNA cargos between Gly-EVs and Neg-CTRL. n=5. (FIG. 15E) Top 10 terms of gene annotation (GO::CC) analysis of RNA cargos significantly enriched in Gly-EVs.

FIGS. 16A-16E show analysis of protein synthesis in recipient neurons resulting from mRNAs delivered through Gly-EVs. (FIG. 16A) Schematic of the experimental workflow for assessing protein synthesis in recipient neurons induced by mRNAs delivered via Gly-EVs. (FIG. 16B) Summary of R-squared values from Hill model fitting, with values greater than 0.8 indicating a good fit. (FIG. 16C) Pie chart showing that 563 mRNAs present in Gly-EVs can lead to corresponding protein synthesis in recipient neurons. (FIG. 16D, FIG. 16E) Top 10 terms of gene annotation (GO::BP (D) and GO::MF (E)) analyses of these 563 mRNAs.

### DETAILED DESCRIPTION

### 1. Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to ±10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

“Antibody” and “antibodies” as used herein refers to antibodies and fragments thereof. For example, “antibody” may refer to monoclonal antibodies, polyclonal antibodies, monospecific antibodies (e.g., which can either be monoclonal, or may also be produced by other means than producing them from a common germ cell), multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, antibody fragments, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence of the present disclosure after aligning the two sequences and introducing gaps, if necessary, to achieve the maximum percent identity. Hence, in case a nucleic acid or protein is longer than a reference sequence, additional nucleotides or amino acids that do not align with the reference sequence are not taken into account for determining sequence identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

### 2. Exosomes

In some aspects, provided herein are exosomes. The terms “exosomes”, “extracellular vesicles” and “EVs” are used interchangeably herein and refer to a family of membrane bound nanoparticles used for therapeutic cargo delivery. Exosomes are enclosed structures surrounded by a membrane (e.g. a lipid bilayer membrane) and are thus capable of carrying various molecules including proteins, DNA, and RNA. Exosomes are extracellular vesicles produced in and released from eukaryotic cells. In some embodiments, the exosomes herein are produced in and released from neurons. In some embodiments, the exosomes herein are produced in and released from neurons, such as by activating NMDA receptors in neurons. For example, in some embodiments the exosomes herein are produced by stimulating neurons with a solution comprising glycine, which activates NMDA receptors and stimulates release of exosomes expressing Notch proteins. In some embodiments, the exosomes herein are released from neurons by stimulating neurons with a magnesium-free glycine solution. The exosomes provided herein find use in delivery of cargo to a desired cell type. Accordingly, the exosomes provided herein are particularly useful in targeted delivery of a therapeutic agent (e.g. proteins, small molecules), which minimizes side effect of the agent that may otherwise occur due to nontargeted delivery.

The exosomes provided herein are predicated at least in part on the surprising discovery that Notch proteins are expressed on the surface of central nervous system (CNS) exosomes (e.g. exosomes produced in and released from neurons). These Notch proteins are shown herein to interact with Notch ligands expressed on the surface of neurons. Interaction between the Notch protein (e.g. receptor) and the Notch ligand is shown herein to lead to selective internalization of the exosomes into neurons expressing the Notch ligand. Disruption of these receptor-ligand interaction disrupts this internalization. Accordingly, the Notch receptor-ligand interaction can be harnessed to generate engineered exosomes for targeted delivery of therapeutic cargo to cells expressing Notch ligands, such as neurons.

In some embodiments, provided herein are exosomes comprising a Notch ligand binding domain. In some embodiments, the Notch ligand binding domain is derived from a Notch receptor. In some embodiments, the Notch ligand binding domain is a synthetic variant of a Notch ligand binding domain of a Notch receptor. Notch ligands include JAG1, JAG2, DLL1, DLL3, and DLL4. The Notch ligand binding domain may bind to any one or more Notch ligands.

The terms “Notch receptor”, “Notch protein”, or “Notch receptor protein” are used interchangeably herein and refer to a family of transmembrane receptor proteins involved in neurogenesis and embryonic development, among other pathways. Mammals possess at least four different Notch receptors, Notch 1, Notch 2, Notch 3, and Notch 4. Generally, Notch receptors comprise a large extracellular domain (ECD), also referred to herein as Notch_{ECD}, a single transmembrane domain, and a small intracellular domain (ICD), also referred to herein as Notch_{ICD}. Binding of a ligand to the ECD induces proteolytic cleavage and release of the intracellular domain, which enters the cell nucleus and modifies gene expression. This ligand binding and subsequent cleavage is also referred to herein as Notch receptor activation, which generates an activated Notch protein (e.g. activated Notch 1, activated Notch 2, etc.).

In some embodiments, the exosomes provided herein comprise a Notch ligand binding domain. The extracellular domain of the Notch receptor comprises the Notch ligand binding domain(s) of the Notch receptor. In some embodiments, the exosomes comprise the extracellular domain of a Notch receptor. In some embodiments, the exosomes comprise at least a portion of the extracellular domain of a Notch receptor. In some embodiments, the exosomes comprise at least a portion of the extracellular domain comprising a Notch ligand binding domain. In some embodiments, the exosomes comprise the extracellular domain and the transmembrane domain of the Notch receptor. In some embodiments, the exosomes comprise the extracellular domain, the transmembrane domain, and at least a portion of the intracellular domain of a Notch receptor. In some embodiments, the exosomes comprise the extracellular domain, the transmembrane domain, and the intracellular domain of the Notch receptor. In some embodiments, the exosomes natively express a wildtype form of a Notch receptor, such as Notch 1, Notch 2, Notch 3, or Notch 4. For example, in some embodiments exosome release from neurons is stimulated, as described herein, and the exosomes are collected natively express a wildtype form of a Notch receptor and are further engineered to contain a cargo. For example, the experiments conducted herein demonstrate that stimulation of exosome release from neurons using a solution comprising glycine (e.g. a magnesium-free glycine solution) causes activation of NMDA receptors and release of exosomes that natively express Notch1 and Notch2. In such embodiments, the exosomes may natively express the wildtype form of the Notch receptor, in which case the exosomes natively express the Notch ligand binding portion of the Notch receptor without further engineering required.

Exemplary sequences for wildtype human Notch proteins are provided below. For the following Notch sequences (i.e. human Notch1, human Notch 2, human Notch3, and human Notch4), the extracellular domain (ECD) is identified in bold, the transmembrane domain is underlined, and the intracellular domain (ICD) is italicized. Notch proteins interact with ligands jagged1 (JAG1), jagged 2 (JAG2), delta like canonical Notch ligands (DLL1, DLL3, and DLL4). The ligand binding domain for JAG1 and JAG1 is shown in bold and underlined, the ligand binding domain for DLL1-4 is bold and italicized.

Human Notch1:
(SEQ ID NO: 1)
MPPLLAPLLCLALLPALAARGPRCSQPGETCLNGGKCEAANGTEA
CVCGGAFVGPRCQDPNPCLSTPCKNAGTCHVVDRRGVADYACSCA
LGFSGPLCLTPLDNACLTNPCRNGGTCDLLTLTEYKCRCPPGWSG
KSCQQADPCASNPCANGGQCLPFEASYICHCPPSFHGPTCRQDVN
ECGQKPGLCRHGGTCHNEVGSYRCVCRATHTGPNCERPYVPCSPS
PCQNGGTCRPTGDVTHECACLPGFTGQNCEENIDDCPGNNCKNGG
ACVDGVNTYNCRCPPEWTGQYCTEDVDECQLMPNACQNGGTCHNT
HGGYNCVCVNGWTGEDCSENIDDCASAACFHGATCHDRVASFYCE
CPHGRTGLLCHLNDACISNPCNEGSNCDTNPVNGKAICTCPSGYT
GPACSQDVDECSLGANPCEHAGKCINTLGSFECQCLQGYTGPRCE
IDVNECVSNPCQNDATCLDQIGEFQCICMPGYEGVHCEVNTDECA
SSPCLHNGRCLDKINEFQCECPTGFTGHLCQYDVDECASTPCKNG
AKCLDGPNTYTCVCTEGYTGTHCEVDIDECDPDPCHYGSCKDGVA
TFTCLCRPGYTGHHCETNINECSSQPCRHGGTCQDRDNAYLCFCL
KGTTGPNCEINLDDCASSPCDSGTCLDKIDGYECACEPGYTGSMC
NINIDECAGNPCHNGGTCEDGINGFTCRCPEGYHDPTCLSEVNEC
NSNPCVHGACRDSLNGYKCDCDPGWSGTNCDINNNECESNPCVNG
GTCKDMTSGYVCTCREGFSGPNCQTNINECASNPCLNQGTCIDDV
AGYKCNCLLPYTGATCEVVLAPCAPSPCRNGGECRQSEDYESFSC
VCPTGWQGQTCEVDINECVLSPCRHGASCQNTHGGYRCHCQAGYS
GRNCETDIDDCRPNPCHNGGSCTDGINTAFCDCLPGFRGTFCEED
INECASDPCRNGANCTDCVDSYTCTCPAGFSGIHCENNTPDCTES
SCFNGGTCVDGINSFTCLCPPGFTGSYCQHDVNECDSQPCLHGGT
CQDGCGSYRCTCPQGYTGPNCQNLVHWCDSSPCKNGGKCWQTHTQ
YRCECPSGWTGLYCDVPSVSCEVAAQRQGVDVARLCQHGGLCVDA
GNTHHCRCQAGYTGSYCEDLVDECSPSPCQNGATCTDYLGGYSCK
CVAGYHGVNCSEEIDECLSHPCQNGGTCLDLPNTYKCSCPRGTQG
VHCEINVDDCNPPVDPVSRSPKCFNNGTCVDQVGGYSCTCPPGFV
GERCEGDVNECLSNPCDARGTQNCVQRVNDFHCECRAGHTGRRCE
SVINGCKGKPCKNGGTCAVASNTARGFICKCPAGFEGATCENDAR
TCGSLRCLNGGTCISGPRSPTCLCLGPFTGPECQFPASSPCLGGN
PCYNQGTCEPTSESPFYRCLCPAKFNGLLCHILDYSFGGGAGRDI
PPPLIEEACELPECQEDAGNKVCSLQCNNHACGWDGGDCSLNFND
PWKNCTQSLQCWKYFSDGHCDSQCNSAGCLFDGFDCQRAEGQCNP
LYDQYCKDHFSDGHCDQGCNSAECEWDGLDCAEHVPERLAAGTLV
VVVLMPPEQLRNSSFHFLRELSRVLHTNVVFKRDAHGQQMIFPYY
GREEELRKHPIKRAAEGWAAPDALLGQVKASLLPGGSEGGRRRRE
LDPMDVRGSIVYLEIDNRQCVQASSQCFQSATDVAAFLGALASLG
SLNIPYKIEAVQSETVEPPPPAQLHFMYVAAAAFVLLFFVGCGVL
LSRKRRRQHGQLWFPEGFKVSEASKKKRREPLGEDSVGLKPLKNA
SDGALMDDNQNEWGDEDLETKKFRFEEPVVLPDLDDQTDHRQWTQ
QHLDAADLRMSAMAPTPPQGEVDADCMDVNVRGPDGFTPLMIASC
SGGGLETGNSEEEEDAPAVISDFIYQGASLHNQTDRTGETALHLA
ARYSRSDAAKRLLEASADANIQDNMGRTPLHAAVSADAQGVFQIL
IRNRATDLDARMHDGTTPLILAARLAVEGMLEDLINSHADVNAVD
DLGKSALHWAAAVNNVDAAVVLLKNGANKDMQNNREETPLFLAAR
EGSYETAKVLLDHFANRDITDHMDRLPRDIAQERMHHDIVRLLDE
YNLVRSPQLHGAPLGGTPTLSPPLCSPNGYLGSLKPGVQGKKVRK
PSSKGLACGSKEAKDLKARRKKSQDGKGCLLDSSGMLSPVDSLES
PHGYLSDVASPPLLPSPFQQSPSVPLNHLPGMPDTHLGIGHLNVA
AKPEMAALGGGGRLAFETGPPRLSHLPVASGTSTVLGSSSGGALN
FTVGGSTSLNGQCEWLSRLQSGMVPNQYNPLRGSVAPGPLSTQAP
SLQHGMVGPLHSSLAASALSQMMSYQGLPSTRLATQPHLVQTQQV
QPQNLQMQQQNLQPANIQQQQSLQPPPPPPQPHLGVSSAASGHLG
RSFLSGEPSQADVQPLGPSSLAVHTILPQESPALPTSLPSSLVPP
VTAAQFLTPPSQHSYSSPVDNTPSHQLQVPEHPFLTPSPESPDQW
SSSSPHSNVSDWSEGVSSPPTSMQSQIARIPEAFK.
Human Notch2:
(SEQ ID NO: 2)
MPALRPALLWALLALWLCCAAPAHALQCRDGYEPCVNEGMCVTYH
NGTGYCKCPEGFLGEYCQHRDPCEKNRCQNGGTCVAQAMLGKATC
RCASGFTGEDCQYSTSHPCFVSRPCLNGGTCHMLSRDTYECTCQV
GFTGKECQWTDACLSHPCANGSTCTTVANQFSCKCLTGFTGQKCE
TDVNECDIPGHCQHGGTCLNLPGSYQCQCPQGFTGQYCDSLYVPC
APSPCVNGGTCRQTGDFTFECNCLPGFEGSTCERNIDDCPNHRCQ
NGGVCVDGVNTYNCRCPPQWTGQFCTEDVDECLLQPNACQNGGTC
ANRNGGYGCVCVNGWSGDDCSENIDDCAFASCTPGSTCIDRVASE
SCMCPEGKAGLLCHLDDACISNPCHKGALCDTNPLNGQYICTCPQ
GYKGADCTEDVDECAMANSNPCEHAGKCVNTDGAFHCECLKGYAG
PRCEMDINECHSDPCQNDATCLDKIGGFTCLCMPGFKGVHCELEI
NECQSNPCVNNGQCVDKVNRFQCLCPPGFTGPVCQIDIDDCSSTP
CLNGAKCIDHPNGYECQCATGFTGVLCEENIDNCDPDPCHHGQCQ
DGIDSYTCICNPGYMGAICSDQIDECYSSPCLNDGRCIDLVNGYQ
CNCQPGTSGVNCEINFDDCASNPCIHGICMDGINRYSCVCSPGFT
GQRCNIDIDECASNPCRKGATCINGVNGFRCICPEGPHHPSCYSQ
VNECLSNPCIHGNCTGGLSGYKCLCDAGWVGINCEVDKNECLSNP
CQNGGTCDNLVNGYRCTCKKGFKGYNCQVNIDECASNPCLNQGTC
FDDISGYTCHCVLPYTGKNCQTVLAPCSPNPCENAAVCKESPNFE
SYTCLCAPGWQGQRCTIDIDECISKPCMNHGLCHNTQGSYMCECP
PGFSGMDCEEDIDDCLANPCQNGGSCMDGVNTFSCLCLPGFTGDK
CQTDMNECLSEPCKNGGTCSDYVNSYTCKCQAGFDGVHCENNINE
CTESSCFNGGTCVDGINSFSCLCPVGFTGSFCLHEINECSSHPCL
NEGTCVDGLGTYRCSCPLGYTGKNCQTLVNLCSRSPCKNKGTCVQ
KKAESQCLCPSGWAGAYCDVPNVSCDIAASRRGVLVEHLCQHSGV
CINAGNTHYCQCPLGYTGSYCEEQLDECASNPCQHGATCSDFIGG
YRCECVPGYQGVNCEYEVDECQNQPCQNGGTCIDLVNHFKCSCPP
GTRGLLCEENIDDCARGPHCLNGGQCMDRIGGYSCRCLPGFAGER
CEGDINECLSNPCSSEGSLDCIQLTNDYLCVCRSAFTGRHCETFV
DVCPQMPCLNGGTCAVASNMPDGFICRCPPGFSGARCQSSCGQVK
CRKGEQCVHTASGPRCFCPSPRDCESGCASSPCQHGGSCHPQRQP
PYYSCQCAPPFSGSRCELYTAPPSTPPATCLSQYCADKARDGVCD
EACNSHACQWDGGDCSLTMENPWANCSSPLPCWDYINNQCDELCN
TVECLFDNFECQGNSKTCKYDKYCADHFKDNHCDQGCNSEECGWD
GLDCAADQPENLAEGTLVIVVLMPPEQLLQDARSFLRALGTLLHT
NLRIKRDSQGELMVYPYYGEKSAAMKKQRMTRRSLPGEQEQEVAG
SKVFLEIDNRQCVQDSDHCFKNTDAAAALLASHAIQGTLSYPLVS
VVSESLTPERTQLLYLLAVAVVIILFIILLGVIMAKRKRKHGSLW
LPEGFTLRRDASNHKRREPVGQDAVGLKNLSVQVSEANLIGTGTS
EHWVDDEGPQPKKVKAEDEALLSEEDDPIDRRPWTQQHLEAADIR
RTPSLALTPPQAEQEVDVLDVNVRGPDGCTPLMLASLRGGSSDLS
DEDEDAEDSSANIITDLVYQGASLQAQTDRTGEMALHLAARYSRA
DAAKRLLDAGADANAQDNMGRCPLHAAVAADAQGVFQILIRNRVT
DLDARMNDGTTPLILAARLAVEGMVAELINCQADVNAVDDHGKSA
LHWAAAVNNVEATLLLLKNGANRDMQDNKEETPLFLAAREGSYEA
AKILLDHFANRDITDHMDRLPRDVARDRMHHDIVRLLDEYNVTPS
PPGTVLTSALSPVICGPNRSFLSLKHTPMGKKSRRPSAKSTMPTS
LPNLAKEAKDAKGSRRKKSLSEKVQLSESSVTLSPVDSLESPHTY
VSDTTSSPMITSPGILQASPNPMLATAAPPAPVHAQHALSFSNLH
EMQPLAHGASTVLPSVSQLLSHHHIVSPGSGSAGSLSRLHPVPVP
ADWMNRMEVNETQYNEMFGMVLAPAEGTHPGIAPQSRPPEGKHIT
TPREPLPPIVTFQLIPKGSIAQPAGAPQPQSTCPPAVAGPLPTMY
QIPEMARLPSVAFPTAMMPQQDGQVAQTILPAYHPFPASVGKYPT
PPSQHSYASSNAAERTPSHSGHLQGEHPYLTPSPESPDQWSSSSP
HSASDWSDVTTSPTPGGAGGGQRGPGTHMSEPPHNNMQVYA
Human Notch3:
(SEQ ID NO: 3)
MGPGARGRRRRRRPMSPPPPPPPVRALPLLLLLAGPGAAAPPCLD
GSPCANGGRCTQLPSREAACLCPPGWVGERCQLEDPCHSGPCAGR
GVCQSSVVAGTARFSCRCPRGFRGPDCSLPDPCLSSPCAHGARCS
VGPDGRFLCSCPPGYQGRSCRSDVDECRVGEPCRHGGTCLNTPGS
FRCQCPAGYTGPLCENPAVPCAPSPCRNGGTCRQSGDLTYDCACL
PGFEGQNCEVNVDDCPGHRCLNGGTCVDGVNTYNCQCPPEWTGQF
CTEDVDECQLQPNACHNGGTCFNTLGGHSCVCVNGWTGESCSQNI
DDCATAVCFHGATCHDRVASFYCACPMGKTGLLCHLDDACVSNPC
HEDAICDTNPVNGRAICTCPPGFTGGACDQDVDECSIGANPCEHL
GRCVNTQGSFLCQCGRGYTGPRCETDVNECLSGPCRNQATCLDRI
GQFTCICMAGFTGTYCEVDIDECQSSPCVNGGVCKDRVNGFSCTC
PSGFSGSTCQLDVDECASTPCRNGAKCVDQPDGYECRCAEGFEGT
LCDRNVDDCSPDPCHHGRCVDGIASFSCACAPGYTGTRCESQVDE
CRSQPCRHGGKCLDLVDKYLCRCPSGTTGVNCEVNIDDCASNPCT
FGVCRDGINRYDCVCQPGFTGPLCNVEINECASSPCGEGGSCVDG
ENGFRCLCPPGSLPPLCLPPSHPCAHEPCSHGICYDAPGGFRCVC
EPGWSGPRCSQSLARDACESQPCRAGGTCSSDGMGFHCTCPPGVQ
GRQCELLSPCTPNPCEHGGRCESAPGQLPVCSCPQGWQGPRCQQD
VDECAGPAPCGPHGICTNLAGSFSCTCHGGYTGPSCDQDINDCDP
NPCLNGGSCQDGVGSFSCSCLPGFAGPRCARDVDECLSNPCGPGT
CTDHVASFTCTCPPGYGGFHCEQDLPDCSPSSCFNGGTCVDGVNS
FSCLCRPGYTGAHCQHEADPCLSRPCLHGGVCSAAHPGFRCTCLE
SFTGPQCQTLVDWCSRQPCQNGGRCVQTGAYCLCPPGWSGRLCDI
RSLPCREAAAQIGVRLEQLCQAGGQCVDEDSSHYCVCPEGRTGSH
CEQEVDPCLAQPCQHGGTCRGYMGGYMCECLPGYNGDNCEDDVDE
CASQPCQHGGSCIDLVARYLCSCPPGTLGVLCEINEDDCGPGPPL
DSGPRCLHNGTCVDLVGGFRCTCPPGYTGLRCEADINECRSGACH
AAHTRDCLQDPGGGFRCLCHAGFSGPRCQTVLSPCESQPCQHGGQ
CRPSPGPGGGLTFTCHCAQPFWGPRCERVARSCRELQCPVGVPCQ
QTPRGPRCACPPGLSGPSCRSFPGSPPGASNASCAAAPCLHGGSC
RPAPLAPFFRCACAQGWTGPRCEAPAAAPEVSEEPRCPRAACQAK
RGDQRCDRECNSPGCGWDGGDCSLSVGDPWRQCEALQCWRLFNNS
RCDPACSSPACLYDNFDCHAGGRERTCNPVYEKYCADHFADGRCD
QGCNTEECGWDGLDCASEVPALLARGVLVLTVLLPPEELLRSSAD
FLQRLSAILRTSLRFRLDAHGQAMVFPYHRPSPGSEPRARRELAP
EVIGSVVMLEIDNRLCLQSPENDHCFPDAQSAADYLGALSAVERL
DFPYPLRDVRGEPLEPPEPSVPLLPLLVAGAVLLLVILVLGVMVA
RRKREHSTLWFPEGFSLHKDVASGHKGRREPVGQDALGMKNMAKG
ESLMGEVATDWMDTECPEAKRLKVEEPGMGAEEAVDCRQWTQHHL
VAADIRVAPAMALTPPQGDADADGMDVNVRGPDGFTPLMLASFCG
GALEPMPTEEDEADDTSASIISDLICQGAQLGARTDRTGETALHL
AARYARADAAKRLLDAGADTNAQDHSGRTPLHTAVTADAQGVFQI
LIRNRSTDLDARMADGSTALILAARLAVEGMVEELIASHADVNAV
DELGKSALHWAAAVNNVEATLALLKNGANKDMQDSKEETPLFLAA
REGSYEAAKLLLDHFANREITDHLDRLPRDVAQERLHQDIVRLLD
QPSGPRSPPGPHGLGPLLCPPGAFLPGLKAAQSGSKKSRRPPGKA
GLGPQGPRGRGKKLTLACPGPLADSSVTLSPVDSLDSPRPFGGPP
ASPGGFPLEGPYAAATATAVSLAQLGGPGRAGLGRQPPGGCVLSL
GLLNPVAVPLDWARLPPPAPPGPSFLLPLAPGPQLLNPGTPVSPQ
ERPPPYLAVPGHGEEYPAAGAHSSPPKARFLRVPSEHPYLTPSPE
SPEHWASPSPPSLSDWSESTPSPATATGAMATTTGALPAQPLPLS
VPSSLAQAQTQLGPQPEVTPKRQVLA
Human Notch4:
(SEQ ID NO: 4)
MQPPSLLLLLLLLLLLCVSVVRPRGLLCGSFPEPCANGGTCLSLS
LGQGTCQCAPGFLGETCQFPDPCQNAQLCQNGGSCQALLPAPLGL
PSSPSPLTPSFLCTCLPGFTGERCQAKLEDPCPPSFCSKRGRCHI
QASGRPQCSCMPGWTGEQCQLRDFCSANPCVNGGVCLATYPQIQC
HCPPGFEGHACERDVNECFQDPGPCPKGTSCHNTLGSFQCLCPVG
QEGPRCELRAGPCPPRGCSNGGTCQLMPEKDSTFHLCLCPPGFIG
PDCEVNPDNCVSHQCQNGGTCQDGLDTYTCLCPETWTGWDCSEDV
DECETQGPPHCRNGGTCQNSAGSFHCVCVSGWGGTSCEENLDDCI
AATCAPGSTCIDRVGSFSCLCPPGRTGLLCHLEDMCLSQPCHGDA
QCSTNPLTGSTLCLCQPGYSGPTCHQDLDECLMAQQGPSPCEHGG
SCLNTPGSFNCLCPPGYTGSRCEADHNECLSQPCHPGSTCLDLLA
TFHCLCPPGLEGQLCEVETNECASAPCLNHADCHDLLNGFQCICL
PGFSGTRCEEDIDECRSSPCANGGQCQDQPGAFHCKCLPGFEGPR
CQTEVDECLSDPCPVGASCLDLPGAFFCLCPSGFTGQLCEVPLCA
PNLCQPKQICKDQKDKANCLCPDGSPGCAPPEDNCTCHHGHCQRS
SCVCDVGWTGPECEAELGGCISAPCAHGGTCYPQPSGYNCTCPTG
YTGPTCSEEMTACHSGPCLNGGSCNPSPGGYYCTCPPSHTGPQCQ
TSTDYCVSAPCENGGTCVNRPGTFSCLCAMGFQGPRCEGKLRPSC
ADSPCRNRATCQDSPQGPRCLCPTGYTGGSCQTLMDLCAQKPCPR
NSHCLQTGPSFHCLCLQGWTGPLCNLPLSSCQKAALSQGIDVSSL
CHNGGLCVDSGPSYFCHCPPGFQGSLCQDHVNPCESRPCQNGATC
MAQPSGYLCQCAPGYDGQNCSKELDACQSQPCHNHGTCTPKPGGF
HCACPPGFVGLRCEGDVDECLDQPCHPTGTAACHSLANAFYCQCL
PGHTGQWCEVEIDPCHSQPCFHGGTCEATAGSPLGFICHCPKGFE
GPTCSHRAPSCGFHHCHHGGLCLPSPKPGFPPRCACLSGYGGPDC
LTPPAPKGCGPPSPCLYNGSCSETTGLGGPGFRCSCPHSSPGPRC
QKPGAKGCEGRSGDGACDAGCSGPGGNWDGGDCSLGVPDPWKGCP
SHSRCWLLFRDGQCHPQCDSEECLFDGYDCETPPACTPAYDQYCH
DHFHNGHCEKGCNTAECGWDGGDCRPEDGDPEWGPSLALLVVLSP
PALDQQLFALARVLSLTLRVGLWVRKDRDGRDMVYPYPGARAEEK
LGGTRDPTYQERAAPQTQPLGKETDSLSAGFVVVMGVDLSRCGPD
HPASRCPWDPGLLLRFLAAMAAVGALEPLLPGPLLAVHPHAGTAP
PANQLPWPVLCSPVAGVILLALGALLVLQLIRRRRREHGALWLPP
GFTRRPRTQSAPHRRRPPLGEDSIGLKALKPKAEVDEDGVVMCSG
PEEGEEVGQAEETGPPSTCQLWSLSGGCGALPQAAMLTPPQESEM
EAPDLDTRGPDGVTPLMSAVCCGEVQSGTFQGAWLGCPEPWEPLL
DGGACPQAHTVGTGETPLHLAARFSRPTAARRLLEAGANPNQPDR
AGRTPLHAAVAADAREVCQLLLRSRQTAVDARTEDGTTPLMLAAR
LAVEDLVEELIAAQADVGARDKWGKTALHWAAAVNNARAARSLLQ
AGADKDAQDNREQTPLFLAAREGAVEVAQLLLGLGAARELRDQAG
LAPADVAHQRNHWDLLTLLEGAGPPEARHKATPGREAGPFPRART
VSVSVPPHGGGALPRCRTLSAGAGPRGGGACLQARTWSVDLAARG
GGAYSHCRSLSGVGAGGGPTPRGRRFSAGMRGPRPNPAIMRGRYG
VAAGRGGRVSTDDWPCDWVALGACGSASNIPIPPPCLTPSPERGS
PQLDCGPPALQEMPINQGGEGKK.

In some embodiments, the exosome comprises a protein having at least 80% identity (e.g. at least 80%, at least 85%, at least 90%, at least 91%, at least 92%, at least 93%, at least 94%, at least 95%, at least 96%, at least 97%, at least 98%, at least 99% identity) with SEQ ID NO: 1, SEQ ID NO: 2, SEQ ID NO: 3 or SEQ ID NO: 4.

In some embodiments, the exosome comprises the extracellular domain of one of SEQ ID NO: 1, SEQ ID NO: 2, SEQ ID NO: 3, or SEQ ID NO: 4. For example, in some embodiments the exosome comprises one of the following:

(SEQ ID NO: 5)
ARGPRCSQPGETCLNGGKCEAANGTEACVCGGAFVGPRCQDPNPC
LSTPCKNAGTCHVVDRRGVADYACSCALGFSGPLCLTPLDNACLT
NPCRNGGTCDLLTLTEYKCRCPPGWSGKSCQQADPCASNPCANGG
QCLPFEASYICHCPPSFHGPTCRQDVNECGQKPGLCRHGGTCHNE
VGSYRCVCRATHTGPNCERPYVPCSPSPCQNGGTCRPTGDVTHEC
ACLPGFTGQNCEENIDDCPGNNCKNGGACVDGVNTYNCRCPPEWT
GQYCTEDVDECQLMPNACQNGGTCHNTHGGYNCVCVNGWTGEDCS
ENIDDCASAACFHGATCHDRVASFYCECPHGRTGLLCHLNDACIS
NPCNEGSNCDTNPVNGKAICTCPSGYTGPACSQDVDECSLGANPC
EHAGKCINTLGSFECQCLQGYTGPRCEIDVNECVSNPCQNDATCL
DQIGEFQCICMPGYEGVHCEVNTDECASSPCLHNGRCLDKINEFQ
CECPTGFTGHLCQYDVDECASTPCKNGAKCLDGPNTYTCVCTEGY
TGTHCEVDIDECDPDPCHYGSCKDGVATFTCLCRPGYTGHHCETN
INECSSQPCRHGGTCQDRDNAYLCFCLKGTTGPNCEINLDDCASS
PCDSGTCLDKIDGYECACEPGYTGSMCNINIDECAGNPCHNGGTC
EDGINGFTCRCPEGYHDPTCLSEVNECNSNPCVHGACRDSLNGYK
CDCDPGWSGTNCDINNNECESNPCVNGGTCKDMTSGYVCTCREGF
SGPNCQTNINECASNPCLNQGTCIDDVAGYKCNCLLPYTGATCEV
VLAPCAPSPCRNGGECRQSEDYESFSCVCPTGWQGQTCEVDINEC
VLSPCRHGASCQNTHGGYRCHCQAGYSGRNCETDIDDCRPNPCHN
GGSCTDGINTAFCDCLPGFRGTFCEEDINECASDPCRNGANCTDC
VDSYTCTCPAGFSGIHCENNTPDCTESSCFNGGTCVDGINSFTCL
CPPGFTGSYCQHDVNECDSQPCLHGGTCQDGCGSYRCTCPQGYTG
PNCQNLVHWCDSSPCKNGGKCWQTHTQYRCECPSGWTGLYCDVPS
VSCEVAAQRQGVDVARLCQHGGLCVDAGNTHHCRCQAGYTGSYCE
DLVDECSPSPCQNGATCTDYLGGYSCKCVAGYHGVNCSEEIDECL
SHPCQNGGTCLDLPNTYKCSCPRGTQGVHCEINVDDCNPPVDPVS
RSPKCFNNGTCVDQVGGYSCTCPPGFVGERCEGDVNECLSNPCDA
RGTQNCVQRVNDFHCECRAGHTGRRCESVINGCKGKPCKNGGTCA
VASNTARGFICKCPAGFEGATCENDARTCGSLRCLNGGTCISGPR
SPTCLCLGPFTGPECQFPASSPCLGGNPCYNQGTCEPTSESPFYR
CLCPAKFNGLLCHILDYSFGGGAGRDIPPPLIEEACELPECQEDA
GNKVCSLQCNNHACGWDGGDCSLNFNDPWKNCTQSLQCWKYFSDG
HCDSQCNSAGCLFDGFDCQRAEGQCNPLYDQYCKDHFSDGHCDQG
CNSAECEWDGLDCAEHVPERLAAGTLVVVVLMPPEQLRNSSFHFL
RELSRVLHTNVVFKRDAHGQQMIFPYYGREEELRKHPIKRAAEGW
AAPDALLGQVKASLLPGGSEGGRRRRELDPMDVRGSIVYLEIDNR
QCVQASSQCFQSATDVAAFLGALASLGSLNIPYKIEAVQSETVEP
PPPAQLH;
(SEQ ID NO: 6)
LQCRDGYEPCVNEGMCVTYHNGTGYCKCPEGFLGEYCQHRDPCEK
NRCQNGGTCVAQAMLGKATCRCASGFTGEDCQYSTSHPCFVSRPC
LNGGTCHMLSRDTYECTCQVGFTGKECQWTDACLSHPCANGSTCT
TVANQFSCKCLTGFTGQKCETDVNECDIPGHCQHGGTCLNLPGSY
QCQCPQGFTGQYCDSLYVPCAPSPCVNGGTCRQTGDFTFECNCLP
GFEGSTCERNIDDCPNHRCQNGGVCVDGVNTYNCRCPPQWTGQFC
TEDVDECLLQPNACQNGGTCANRNGGYGCVCVNGWSGDDCSENID
DCAFASCTPGSTCIDRVASFSCMCPEGKAGLLCHLDDACISNPCH
KGALCDTNPLNGQYICTCPQGYKGADCTEDVDECAMANSNPCEHA
GKCVNTDGAFHCECLKGYAGPRCEMDINECHSDPCQNDATCLDKI
GGFTCLCMPGFKGVHCELEINECQSNPCVNNGQCVDKVNRFQCLC
PPGFTGPVCQIDIDDCSSTPCLNGAKCIDHPNGYECQCATGFTGV
LCEENIDNCDPDPCHHGQCQDGIDSYTCICNPGYMGAICSDQIDE
CYSSPCLNDGRCIDLVNGYQCNCQPGTSGVNCEINFDDCASNPCI
HGICMDGINRYSCVCSPGFTGQRCNIDIDECASNPCRKGATCING
VNGFRCICPEGPHHPSCYSQVNECLSNPCIHGNCTGGLSGYKCLC
DAGWVGINCEVDKNECLSNPCQNGGTCDNLVNGYRCTCKKGFKGY
NCQVNIDECASNPCLNQGTCFDDISGYTCHCVLPYTGKNCQTVLA
PCSPNPCENAAVCKESPNFESYTCLCAPGWQGQRCTIDIDECISK
PCMNHGLCHNTQGSYMCECPPGFSGMDCEEDIDDCLANPCQNGGS
CMDGVNTFSCLCLPGFTGDKCQTDMNECLSEPCKNGGTCSDYVNS
YTCKCQAGFDGVHCENNINECTESSCFNGGTCVDGINSFSCLCPV
GFTGSFCLHEINECSSHPCLNEGTCVDGLGTYRCSCPLGYTGKNC
QTLVNLCSRSPCKNKGTCVQKKAESQCLCPSGWAGAYCDVPNVSC
DIAASRRGVLVEHLCQHSGVCINAGNTHYCQCPLGYTGSYCEEQL
DECASNPCQHGATCSDFIGGYRCECVPGYQGVNCEYEVDECQNQP
CQNGGTCIDLVNHFKCSCPPGTRGLLCEENIDDCARGPHCLNGGQ
CMDRIGGYSCRCLPGFAGERCEGDINECLSNPCSSEGSLDCIQLT
NDYLCVCRSAFTGRHCETFVDVCPQMPCLNGGTCAVASNMPDGFI
CRCPPGFSGARCQSSCGQVKCRKGEQCVHTASGPRCFCPSPRDCE
SGCASSPCQHGGSCHPQRQPPYYSCQCAPPFSGSRCELYTAPPST
PPATCLSQYCADKARDGVCDEACNSHACQWDGGDCSLTMENPWAN
CSSPLPCWDYINNQCDELCNTVECLFDNFECQGNSKTCKYDKYCA
DHFKDNHCDQGCNSEECGWDGLDCAADQPENLAEGTLVIVVLMPP
EQLLQDARSFLRALGTLLHTNLRIKRDSQGELMVYPYYGEKSAAM
KKQRMTRRSLPGEQEQEVAGSKVFLEIDNRQCVQDSDHCFKNTDA
AAALLASHAIQGTLSYPLVSVVSESLTPERTQ;
(SEQ ID NO: 7)
PPCLDGSPCANGGRCTQLPSREAACLCPPGWVGERCQLEDPCHSG
PCAGRGVCQSSVVAGTARFSCRCPRGFRGPDCSLPDPCLSSPCAH
GARCSVGPDGRFLCSCPPGYQGRSCRSDVDECRVGEPCRHGGTCL
NTPGSFRCQCPAGYTGPLCENPAVPCAPSPCRNGGTCRQSGDLTY
DCACLPGFEGQNCEVNVDDCPGHRCLNGGTCVDGVNTYNCQCPPE
WTGQFCTEDVDECQLQPNACHNGGTCFNTLGGHSCVCVNGWTGES
CSQNIDDCATAVCFHGATCHDRVASFYCACPMGKTGLLCHLDDAC
VSNPCHEDAICDTNPVNGRAICTCPPGFTGGACDQDVDECSIGAN
PCEHLGRCVNTQGSFLCQCGRGYTGPRCETDVNECLSGPCRNQAT
CLDRIGQFTCICMAGFTGTYCEVDIDECQSSPCVNGGVCKDRVNG
FSCTCPSGFSGSTCQLDVDECASTPCRNGAKCVDQPDGYECRCAE
GFEGTLCDRNVDDCSPDPCHHGRCVDGIASFSCACAPGYTGTRCE
SQVDECRSQPCRHGGKCLDLVDKYLCRCPSGTTGVNCEVNIDDCA
SNPCTFGVCRDGINRYDCVCQPGFTGPLCNVEINECASSPCGEGG
SCVDGENGFRCLCPPGSLPPLCLPPSHPCAHEPCSHGICYDAPGG
FRCVCEPGWSGPRCSQSLARDACESQPCRAGGTCSSDGMGFHCTC
PPGVQGRQCELLSPCTPNPCEHGGRCESAPGQLPVCSCPQGWQGP
RCQQDVDECAGPAPCGPHGICTNLAGSFSCTCHGGYTGPSCDQDI
NDCDPNPCLNGGSCQDGVGSFSCSCLPGFAGPRCARDVDECLSNP
CGPGTCTDHVASFTCTCPPGYGGFHCEQDLPDCSPSSCFNGGTCV
DGVNSFSCLCRPGYTGAHCQHEADPCLSRPCLHGGVCSAAHPGFR
CTCLESFTGPQCQTLVDWCSRQPCQNGGRCVQTGAYCLCPPGWSG
RLCDIRSLPCREAAAQIGVRLEQLCQAGGQCVDEDSSHYCVCPEG
RTGSHCEQEVDPCLAQPCQHGGTCRGYMGGYMCECLPGYNGDNCE
DDVDECASQPCQHGGSCIDLVARYLCSCPPGTLGVLCEINEDDCG
PGPPLDSGPRCLHNGTCVDLVGGFRCTCPPGYTGLRCEADINECR
SGACHAAHTRDCLQDPGGGFRCLCHAGFSGPRCQTVLSPCESQPC
QHGGQCRPSPGPGGGLTFTCHCAQPFWGPRCERVARSCRELQCPV
GVPCQQTPRGPRCACPPGLSGPSCRSFPGSPPGASNASCAAAPCL
HGGSCRPAPLAPFFRCACAQGWTGPRCEAPAAAPEVSEEPRCPRA
ACQAKRGDQRCDRECNSPGCGWDGGDCSLSVGDPWRQCEALQCWR
LFNNSRCDPACSSPACLYDNFDCHAGGRERTCNPVYEKYCADHFA
DGRCDQGCNTEECGWDGLDCASEVPALLARGVLVLTVLLPPEELL
RSSADFLQRLSAILRTSLRFRLDAHGQAMVFPYHRPSPGSEPRAR
RELAPEVIGSVVMLEIDNRLCLQSPENDHCFPDAQSAADYLGALS
AVERLDFPYPLRDVRGEPLEPPEPSVPL;
or
(SEQ ID NO: 8)
RGLLCGSFPEPCANGGTCLSLSLGQGTCQCAPGFLGETCQFPDPC
QNAQLCQNGGSCQALLPAPLGLPSSPSPLTPSFLCTCLPGFTGER
CQAKLEDPCPPSFCSKRGRCHIQASGRPQCSCMPGWTGEQCQLRD
FCSANPCVNGGVCLATYPQIQCHCPPGFEGHACERDVNECFQDPG
PCPKGTSCHNTLGSFQCLCPVGQEGPRCELRAGPCPPRGCSNGGT
CQLMPEKDSTFHLCLCPPGFIGPDCEVNPDNCVSHQCQNGGTCQD
GLDTYTCLCPETWTGWDCSEDVDECETQGPPHCRNGGTCQNSAGS
FHCVCVSGWGGTSCEENLDDCIAATCAPGSTCIDRVGSFSCLCPP
GRTGLLCHLEDMCLSQPCHGDAQCSTNPLTGSTLCLCQPGYSGPT
CHQDLDECLMAQQGPSPCEHGGSCLNTPGSFNCLCPPGYTGSRCE
ADHNECLSQPCHPGSTCLDLLATFHCLCPPGLEGQLCEVETNECA
SAPCLNHADCHDLLNGFQCICLPGFSGTRCEEDIDECRSSPCANG
GQCQDQPGAFHCKCLPGFEGPRCQTEVDECLSDPCPVGASCLDLP
GAFFCLCPSGFTGQLCEVPLCAPNLCQPKQICKDQKDKANCLCPD
GSPGCAPPEDNCTCHHGHCQRSSCVCDVGWTGPECEAELGGCISA
PCAHGGTCYPQPSGYNCTCPTGYTGPTCSEEMTACHSGPCLNGGS
CNPSPGGYYCTCPPSHTGPQCQTSTDYCVSAPCFNGGTCVNRPGT
FSCLCAMGFQGPRCEGKLRPSCADSPCRNRATCQDSPQGPRCLCP
TGYTGGSCQTLMDLCAQKPCPRNSHCLQTGPSFHCLCLQGWTGPL
CNLPLSSCQKAALSQGIDVSSLCHNGGLCVDSGPSYFCHCPPGFQ
GSLCQDHVNPCESRPCQNGATCMAQPSGYLCQCAPGYDGQNCSKE
LDACQSQPCHNHGTCTPKPGGFHCACPPGFVGLRCEGDVDECLDQ
PCHPTGTAACHSLANAFYCQCLPGHTGQWCEVEIDPCHSQPCFHG
GTCEATAGSPLGFICHCPKGFEGPTCSHRAPSCGFHHCHHGGLCL
PSPKPGFPPRCACLSGYGGPDCLTPPAPKGCGPPSPCLYNGSCSE
TTGLGGPGFRCSCPHSSPGPRCQKPGAKGCEGRSGDGACDAGCSG
PGGNWDGGDCSLGVPDPWKGCPSHSRCWLLFRDGQCHPQCDSEEC
LFDGYDCETPPACTPAYDQYCHDHFHNGHCEKGCNTAECGWDGGD
CRPEDGDPEWGPSLALLVVLSPPALDQQLFALARVLSLTLRVGLW
VRKDRDGRDMVYPYPGARAEEKLGGTRDPTYQERAAPQTQPLGKE
TDSLSAGFVVVMGVDLSRCGPDHPASRCPWDPGLLLRFLAAMAAV
GALEPLLPGPLLAVHPHAGTAPPANQLPW.

In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with SEQ ID NO: 5, SEQ ID NO: 6, SEQ ID NO: 7, or SEQ ID NO: 8.

In some embodiments, the exosome comprises a Notch ligand binding domain (also referred to herein as a Notch ligand binding portion) of one of SEQ ID NO: 1, SEQ ID NO: 2, SEQ ID NO: 3, or SEQ ID NO: 4. In some embodiments, the Notch ligand binding domain is expressed on the extracellular surface of the exosome. For example, in some embodiments the exosome comprises (e.g. expresses extracellularly) one or more of:

(SEQ ID NO: 9)
DVDECQLMPNACQNGGTCHNTHGGYNCVCVNGWTGEDCSENIDDC
ASAACFHGATCHDRVASFYCECPHGRTGLLCHLNDACISNPCNEG
SNCDTNPVNGKAICTCPSGYTGPACSQDVDECSLG,
(SEQ ID NO: 10)
ANPCEHAGKCINTLGSFECQCLQGYTGPRCEID,
and
(SEQ ID NO: 11)
VNECVSNPCQNDATCLDQIGEFQCICMPGYEGVHCEVNTDECASS
PCLHNGRCLDKINEFQCECPTGFTGHLCQ.

In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with the sequence of SEQ ID NO: 9, SEQ ID NO: 10, SEQ ID NO: 11, or a combination thereof. In some embodiments, the exosome comprises the polypeptide of SEQ ID NO: 9. In some embodiments, the exosome comprises the polypeptide of SEQ ID NO: 11. In some embodiments, the exosome comprises the polypeptide of SEQ ID NO: 9 and the polypeptide of SEQ ID NO: 11. In some embodiments, the exosome comprises the polypeptide of SEQ ID NO: 10. In some embodiments, the exosome comprises DVDECQLMPNACQNGGTCHNTHGGYNCVCVNGWTGEDCSENIDDCASAACFHGATC HDRVASFYCECPHGRTGLLCHLNDACISNPCNEGSNCDTNPVNGKAICTCPSGYTGPAC SQDVDECSLGANPCEHAGKCINTLGSFECQCLQGYTGPRCEIDVNECVSNPCQNDATCL DQIGEFQCICMPGYEGVHCEVNTDECASSPCLHNGRCLDKINEFQCECPTGFTGHLCQ (SEQ ID NO: 12). In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with SEQ ID NO: 12.

As another example, in some embodiments the exosome comprises (e.g. expresses extracellularly) one or more of:

(SEQ ID NO: 13)
DVDECLLQPNACQNGGTCANRNGGYGCVCVNGWSGDDCSENIDDC
AFASCTPGSTCIDRVASFSCMCPEGKAGLLCHLDDACISNPCHKG
ALCDTNPLNGQYICTCPQGYKGADCTE,
(SEQ ID NO: 14)
DVDECAMANSNPCEHAGKCVNTDGAFHCECLKGYAGPRCEMDINE
CHSDPCQNDATCLDKIGGFTCLCMPGFKGVHCE,
and
(SEQ ID NO: 15)
LEINECQSNPCVNNGQCVDKVNRFQCLCPPGFTGPVCQ.

In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with the sequence of SEQ ID NO: 13, SEQ ID NO: 14, SEQ ID NO: 15, or a combination thereof. In some embodiments, the exosome comprises the polypeptide of SEQ ID NO: 13. In some embodiments, the exosome comprises the polypeptide of SEQ ID NO: 15. In some embodiments, the exosome comprises the polypeptide of SEQ ID NO: 13 and the polypeptide of SEQ ID NO: 15. In some embodiments, the exosome comprises the polypeptide of SEQ ID NO: 14. In some embodiments, the exosome comprises DVDECLLQPNACQNGGTCANRNGGYGCVCVNGWSGDDCSENIDDCAFASCTPGSTCID RVASFSCMCPEGKAGLLCHLDDACISNPCHKGALCDTNPLNGQYICTCPQGYKGADCT EDVDECAMANSNPCEHAGKCVNTDGAFHCECLKGYAGPRCEMDINECHSDPCQNDAT CLDKIGGFTCLCMPGFKGVHCELEINECQSNPCVNNGQCVDKVNRFQCLCPPGFTGPVC Q (SEQ ID NO: 16). In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with SEQ ID NO: 16.

As another example, in some embodiments the exosome comprises (e.g. expresses extracellularly) one or more of:

(SEQ ID NO: 17)
NIDDCATAVCFHGATCHDRVASFYCACPMGKTGLLCHLDDACVSN
PCHEDAICDTNPVNGRAICTCPPGFTGGACDQDVDECSIGANPCE
HLGRCVNTQGSFLCQCGRGYTGPRCET,
(SEQ ID NO: 18)
DVNECLSGPCRNQATCLDRIGQFTCICMAGFTGTYCEVDIDECQS
SPCVNGGVCKDRVNGFSCTCPSGFSGSTCQ,
and
(SEQ ID NO: 19)
LDVDECASTPCRNGAKCVDQPDGYECRCAEGFEGTLCD

In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with the sequence of SEQ ID NO: 17, SEQ ID NO: 18, SEQ ID NO: 19, or a combination thereof. In some embodiments, the exosome comprises the polypeptide of SEQ ID NO: 17. In some embodiments, the exosome comprises the polypeptide of SEQ ID NO: 19. In some embodiments, the exosome comprises the polypeptide of SEQ ID NO: 17 and the polypeptide of SEQ ID NO: 19. In some embodiments, the exosome comprises the polypeptide of SEQ ID NO: 18. In some embodiments, the exosome comprises NIDDCATAVCFHGATCHDRVASFYCACPMGKTGLLCHLDDACVSNPCHEDAICDTNPV NGRAICTCPPGFTGGACDQDVDECSIGANPCEHLGRCVNTQGSFLCQCGRGYTGPRCET DVNECLSGPCRNQATCLDRIGQFTCICMAGFTGTYCEVDIDECQSSPCVNGGVCKDRVN GFSCTCPSGFSGSTCQLDVDECASTPCRNGAKCVDQPDGYECRCAEGFEGTLCD (SEQ ID NO: 20). In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with SEQ ID NO: 20.

As another example, in some embodiments the exosome comprises (e.g. expresses extracellularly) one or more of:

DVDECETQGPPHCRNGGTCQNSAGSFHCVCVSGWGGTSCEENLDDCIAATCAP GSTCIDRVGSFSCLCPPGRTGLLCHLEDMCLSQPCHGDAQCSTNPLTGSTLCLCQPGYSG PTCHQ (SEQ ID NO: 21), DLDECLMAQQGPSPCEHGGSCLNTPGSFNCLCPPGYTGSRCEADHNECLSQPCHPGSTC LDLLATFHCLCPPGLEGQLCE (SEQ ID NO: 22), and VETNECASAPCLNHADCHDLLNGFQCICLPGFSGTRCE (SEQ ID NO: 23). In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with the sequence of SEQ ID NO: 21, SEQ ID NO: 22, SEQ ID NO: 23, or a combination thereof. In some embodiments, the exosome comprises the polypeptide of SEQ ID NO: 21. In some embodiments, the exosome comprises the polypeptide of SEQ ID NO: 23. In some embodiments, the exosome comprises the polypeptide of SEQ ID NO: 21 and the polypeptide of SEQ ID NO: 23. In some embodiments, the exosome comprises the polypeptide of SEQ ID NO: 22. In some embodiments, the exosome comprises DVDECETQGPPHCRNGGTCQNSAGSFHCVCVSGWGGTSCEENLDDCIAATCAPGSTCID RVGSFSCLCPPGRTGLLCHLEDMCLSQPCHGDAQCSTNPLTGSTLCLCQPGYSGPTCHQ DLDECLMAQQGPSPCEHGGSCLNTPGSFNCLCPPGYTGSRCEADHNECLSQPCHPGSTC LDLLATFHCLCPPGLEGQLCEVETNECASAPCLNHADCHDLLNGFQCICLPGFSGTRCE (SEQ ID NO: 24). In some embodiments, the exosome comprises a polypeptide having at least 80% sequence identity (e.g. at least 80%, at least 85%, at least 90%, at least 91%, at least 92%, at least 93%, at least 94%, at least 95%, at least 96%, at least 97%, at least 98%, at least 99% identity) with SEQ ID NO: 24.

In some embodiments, the Notch ligand binding domain is a synthetic polypeptide that mimics a ligand binding domain of a wildtype Notch receptor. For example, in some embodiments the Notch ligand binding domain is a synthetic polypeptide that mimics the ligand binding domain of a wildtype Notch receptor and effectively binds to one or more Notch ligands (e.g. JAG1, JAG2, DLL1, DLL3, or DLL4). For example, in some embodiments the Notch ligand binding domain is a synthetic polypeptide containing one or more mutations in one or more domains relative to the wildtype form of the Notch receptor. For example, one or more mutations may be made in the extracellular domain compared to the wildtype Notch receptor to improve affinity for a Notch ligand. The Notch receptor may be Notch 1, Notch 2, Notch 3, or Notch 4. In some embodiments, the Notch ligand binding domain comprises an amino acid sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) with the amino acid sequence of an extracellular domain of a wildtype Notch receptor. In some embodiments, the ligand binding portion of the Notch receptor comprises an amino acid sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) with the amino acid sequence of a Notch ligand binding domain of a wildtype Notch receptor. In some embodiments, one or more ligand-binding domains from alternative receptor proteins are incorporated into the exosome-targeting/transmembrane sequence of the Notch receptor, thereby enabling a significantly broader range or even more discrete cell-type-specific cargo delivery. The Notch receptor may be from any suitable species, including humans, rodents, or other non-human mammals.

### 3. Cargo

The exosomes further comprise a therapeutic cargo. The therapeutic cargo may be any suitable cargo for which targeted delivery to neurons is desirable. For example, in some embodiments the therapeutic cargo is an agent for the treatment of a neurological disease or condition. For example, in some embodiments the therapeutic cargo is an agent for treatment of a neurological disease or condition such as a lysosomal storage disease, Parkinson's disease, Huntington's disease, Alzheimer's disease, multiple sclerosis, ALS, etc. In some embodiments, the therapeutic cargo is an agent for treatment of an injury of the central nervous system, such as a traumatic injury or a non-traumatic injury caused by stroke, ischemia, and the like. As another example, in some embodiments the therapeutic cargo is an agent for treatment of a cancer of the central nervous system (e.g. cancer in the brain or spinal cord). The cancer of the central nervous system may have originated in the central nervous system (e.g. may be a primary central nervous system cancer) or may be a different cancer type that has metastasized to the central nervous system. For example, the cancer of the central nervous system may be an astrocytoma, a glioblastoma, an oligodendroglioma, a medulloblastoma, an ependymoma, a lymphoma, a meningioma, or a cancer that has metastasized to the brain or the spinal cord. In some embodiments, the therapeutic cargo is a nucleic acid (e.g. DNA, RNA, mRNA, siRNA, shRNA, etc.), a peptide, a protein, an antibody (including antibody fragments, antibody-like molecules, antibody derivatives, etc.), an aptamer, a carbohydrate, a lipid, or a small molecule. In some embodiments, the therapeutic cargo is a nucleic acid. The experiments herein demonstrate that the exosomes provided herein are able to carry nucleic acid cargo (e.g. mRNA) and enter into neurons wherein the nucleic acid cargo is translated into a functional protein. As such, in some embodiments the therapeutic cargo may comprise a suitable nucleic acid therapy (e.g. DNA, RNA, mRNA, siRNA, shRNA, antisense oligonucleotide, etc.) for treatment of the disease or condition for which targeted delivery to neurons is beneficial (e.g. the neurological disease or condition, the cancer of the central nervous system). The therapeutic cargo may be loaded into the exosome by any suitable method, including, for example, electroporation, sonication, freeze-thaw cycles, extrusion, transfection, and endogenous loading, particularly transfection and endogenous loading. The therapeutic cargo can be loaded by a method suitable for the cargo itself.

### 4. Engineered Exosome

An exosome loaded with a therapeutic cargo is also referred to herein as an “engineered” exosome. In some embodiments, the exosomes used to generate the engineered exosomes herein are derived from the central nervous system. For example, in some embodiments the exosomes are derived from neurons. Neurons may be stimulated to release exosomes, which can be isolated and engineered to express the desired receptor and/or cargo. For example, neurons can be stimulated to release exosomes by activating synaptic N-methyl-D-aspartate receptors (NMDARs). In some embodiments, exosome release is triggered by activating NMDARs with glycine, as described in the accompanying Examples. For example, in some embodiments exosome release is triggered treating neurons with a magnesium-free glycine solution. In some embodiments, the solution comprises 1 uM to 100 mM glycine.

In some embodiments, provided herein are methods comprising providing to a subject an engineered exosome described herein. For example, an engineered exosome comprising a therapeutic agent for treatment of a neurological disease or condition can be provided to a subject having or suspected of having the neurological disease or condition. For example, anticancer agents for cancers of the central nervous system (e.g. brain cancer) may be particularly well-suited for use in the exosomes described herein, wherein delivery of the anticancer agent selectively to neurons would achieve a localized delivery of the anticancer agent to the target tissue and thus avoid unwanted side effects associated with systemic delivery to the subject. As another example, diseases of the central nervous system are often difficult to treat due to the inability of therapeutic agents for the disease to cross the blood-brain barrier (BBB). In contrast, exosomes expressing a ligand binding portion of a Notch receptor, as described herein, are particularly useful due to their ability to cross the BBB and selectively deliver a therapeutic cargo to neurons. For example, the exosomes described herein may be particularly useful for treatment of neurological diseases including lysosomal storage diseases such as glucocerebrosidase for Gaucher disease, Parkinson's disease, Alzheimer's disease, amyotrophic lateral sclerosis (ALS), multiple sclerosis (MS), epilepsy, Guillain-Barré syndrome (GBS), and the like.

The engineered exosomes herein may be provided to the subject by any suitable administration route. Administration may be systemically/peripherally or at the site of desired action, including but not limited to, oral (e.g., by ingestion); topical (including e.g. transdermal, intranasal, ocular, buccal, and sublingual); pulmonary (e.g., by inhalation or insufflation therapy using, e.g., an aerosol, e.g., through mouth or nose); rectal; vaginal; parenteral (e.g., by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal injection); or by implant of a depot, for example, subcutaneously or intramuscularly.

In some embodiments, the engineered exosome is comprised in a composition (e.g. a pharmaceutical composition) comprising a pharmaceutically acceptable carrier or excipient. Reference to providing or administering an engineered exosome to the subject is inclusive of providing or administering a composition (e.g. a pharmaceutical composition) comprising the engineered exosome to the subject. The phrase “pharmaceutically acceptable,” as used in connection with compositions of the present disclosure, refers to molecular entities and other ingredients of such compositions that are physiologically tolerable and do not typically produce undesirable reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. The pharmaceutically acceptable carrier should also be compatible with the active ingredient of the composition (e.g., the engineered exosome). Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The dose of the engineered exosome provided to the subject may depend on the severity of the condition, the individual patient parameters including age, physical condition, size, gender and weight, the duration of the treatment, the nature of concurrent therapy (if any), the specific route of administration, the precise therapeutic cargo used, and like factors within the knowledge and expertise of the health practitioner. In some embodiments, the effective amount is sufficient to treat a disease or condition (e.g. the neurological disease, the central nervous system injury, the central nervous system cancer) in the subject. For example, in some embodiments the effective amount alleviates, relieves, ameliorates, improves, reduces the symptoms, or delays the progression of the disease or condition in the subject.

It will be appreciated that appropriate dosages can vary from patient to patient. Determining the optimal dosage will generally involve the balancing of the level of therapeutic benefit against any risk or deleterious side effects of the treatments of the present disclosure. The amount and route of administration will ultimately be at the discretion of the physician, although generally the dosage will be to achieve local concentrations at the site of action which achieve the desired effect without causing substantial harmful or deleterious side-effects.

The engineered exosome may be provided to the subject in be in a single dose or in multiple doses throughout the course of treatment. Methods of determining the most effective means and dosage of administration are well known to those of skill in the art and will vary with the exact therapeutic cargo and route of administration used for therapy, the severity of the disease or condition, and the subject being treated. Single or multiple administrations can be carried out with the dose level and pattern being selected by the treating physician.

In some embodiments, a given dose is provided to the subject continuously or intermittently over the course of a suitable dosing window. For example, the dosing window may be 10 minutes to 6 hours, 20 minutes to 5 hours, 30 minutes to 4 hours, or about 1 to 3 hours. In some embodiments, the engineered exosome is provided to the subject once per day. In some embodiments, the engineered exosome is provided to the subject multiple times per day. In some embodiments, the engineered exosome is provided to the subject every other day, every 3 days, every 4 days, every 5 days, every 6 days, every 7 days, every 8 days, every 9 days, every 10 days, every 2 weeks, every 3 weeks, monthly, every 2 months, every 3 months, every 4 months, every 5 months, every 6 months, annually, etc. The engineered exosome may be administered until a desired reduction of symptoms is achieved.

In some embodiments, the engineered exosome is provided to the subject in combination with other therapies for the disease or condition (e.g. with other therapies for the neurological disease, other therapies for the cancer). Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

### EXAMPLES

### Example 1

Extracellular vesicles (EVs) facilitate intercellular communication by transferring cargo between cells in a variety of tissues. However, how EVs achieve cell type-specific intercellular communication is still largely unknown. Herein it is demonstrated that Notch1 and Notch2 proteins are expressed on the surface of neuronal EVs that have been generated in response to neuronal excitatory synaptic activity. Notch ligands bind these EVs on the neuronal plasma membrane, trigger their internalization, activate the Notch signaling pathway, and drive the expression of Notch target genes. The generation of these neuronal EVs requires the ESCRT-associated protein Alix. Adult Alix conditional knockout mice have reduced hippocampal Notch signaling activation and glutamatergic synaptic protein expression. Thus, EVs facilitate neuron to neuron communication via the Notch receptor-ligand system in the brain.

### Results

### NMDAR Activity-Induced EVs Contain Notch1 and Notch2 Proteins

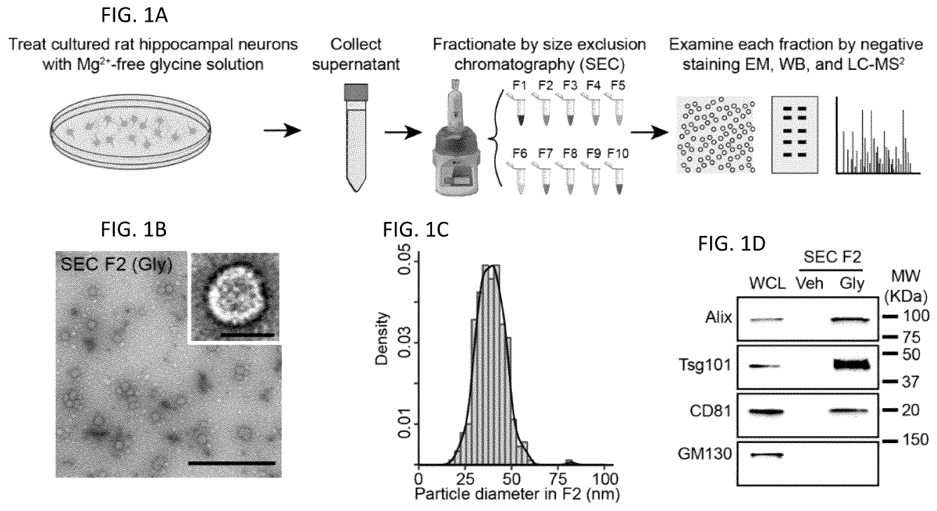

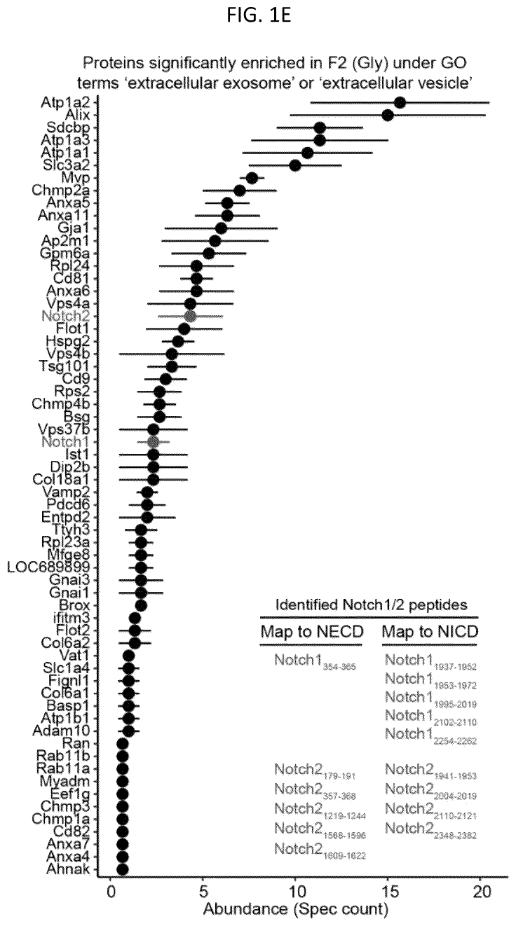

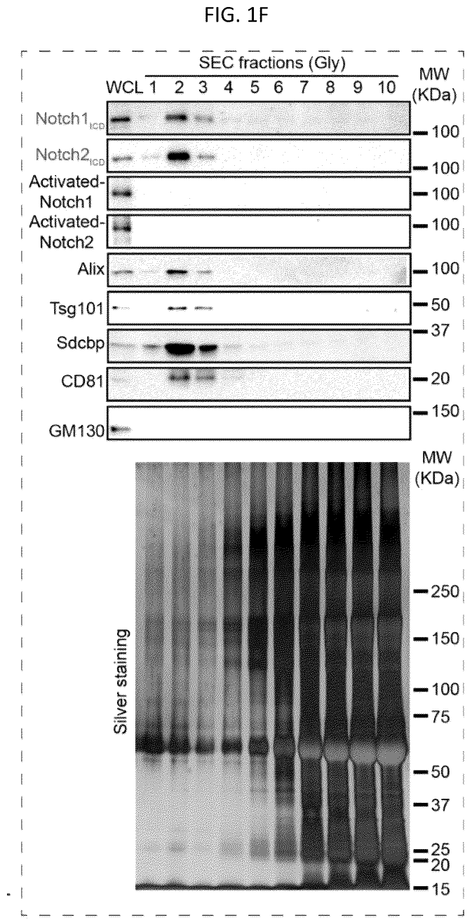

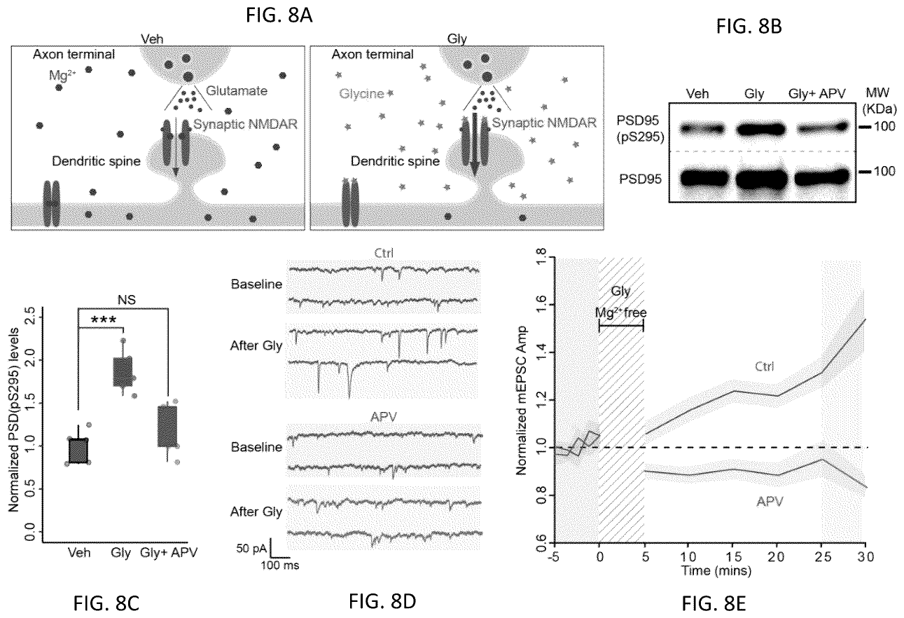

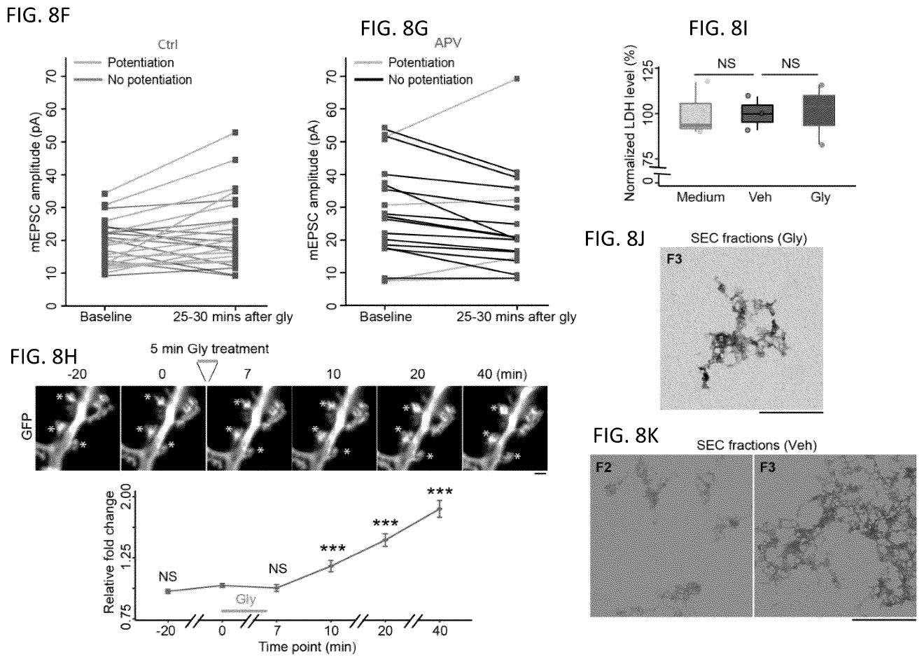

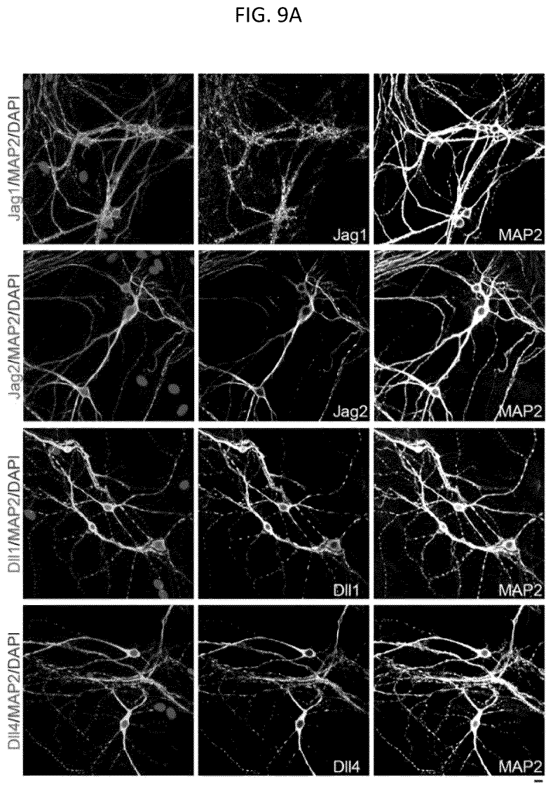

Experiments were conducted to identify proteins responsible for mediating cell type-specific EV communication using cultured rodent neurons. Neuronal EV biogenesis and release was triggered by activating synaptic N-methyl-D-aspartate receptors (NMDARs) with a Mg²⁺-free glycine solution¹⁶ (FIGS. 1A and 8A-I). Neuronal EVs were isolated using a previously developed isolation strategy based on size exclusion chromatography (SEC) 17 (FIG. 1A). Negative staining electron microscopy (EM) analysis revealed abundant EV-like particles in the second SEC fraction isolated from the Mg²⁺-free glycine solution used to stimulate the neurons (FIGS. 1B & C, and 8J & K). Western blot (WB) analysis confirmed that this fraction was enriched for three EV markers Alix, Tsg101, and CD81 but not for GM130, a protein located in the Golgi apparatus (FIG. 1D).

All ten SEC fractions were individually analyzed with liquid chromatography tandem mass spectrometry (LC-MS/MS)-based proteomics (FIG. 1E). 114 proteins with significantly higher levels in EV-enriched fraction, relative to the other fractions were identified. Gene ontology enrichment analysis revealed robust enrichment of proteins associated with EVs, including 60 canonical EV markers (e.g., ESCRT complex proteins and tetraspanins). Among the additional 54 proteins, Notch1 and Notch2 stood out as prominent candidates for specifying EV binding, because they are transmembrane proteins that are captured by high affinity ligands. Notably, the identified Notch1 and Notch 2 peptides mapped to both the extra- and intra-cellular domains (ECD or ICD, respectively). Thus Notch1 and Notch2 in these EVs have not been activated by proteolytic cleavage (FIG. 1E). WB analysis of SEC fractions confirmed the presence of Notch1_{ICD} and Notch2_{ICD} in EVs but failed to detect activated Notch1, activated Notch2, or the Notch ligands Jag1, Jag2, Dll1 and Dll4 (FIG. 1F).

### Neurons Internalize EVs Using Notch Ligands

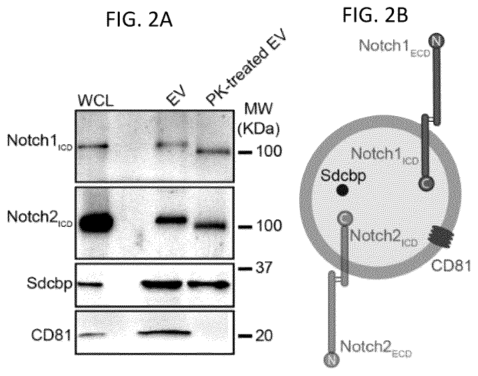

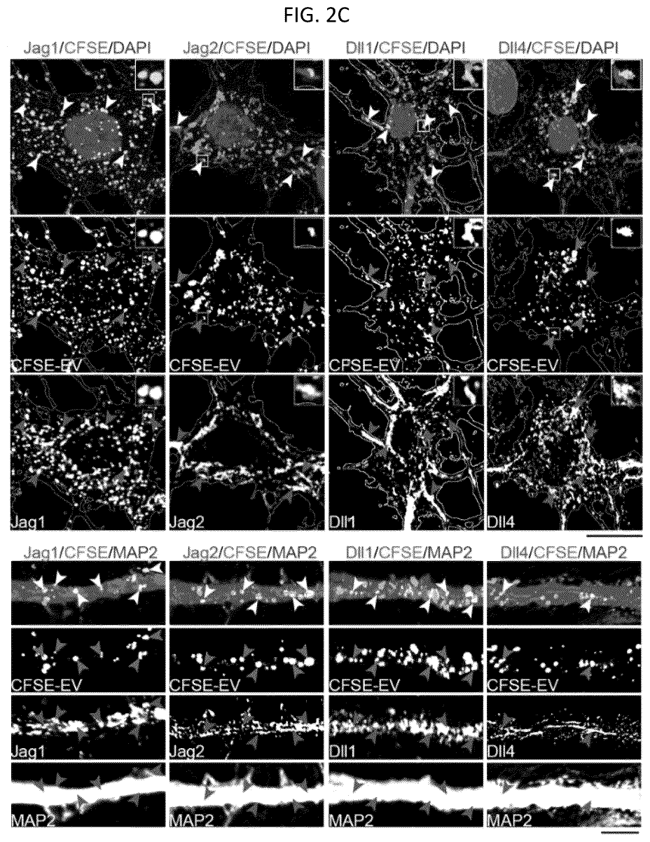

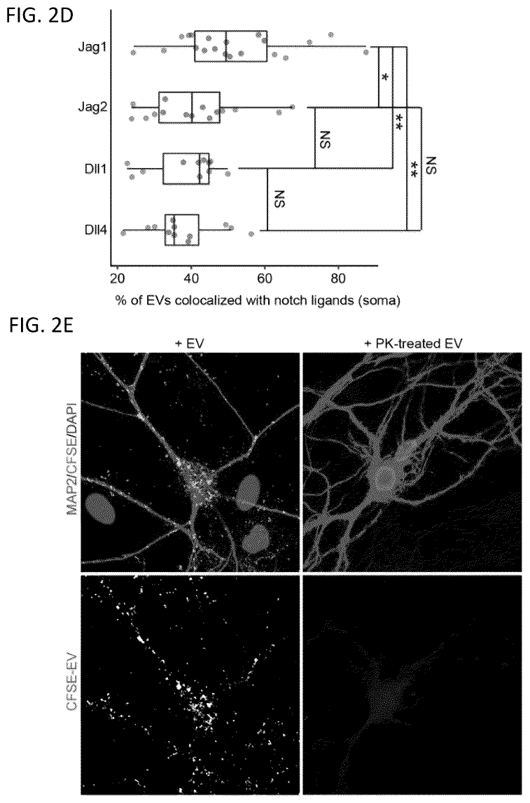

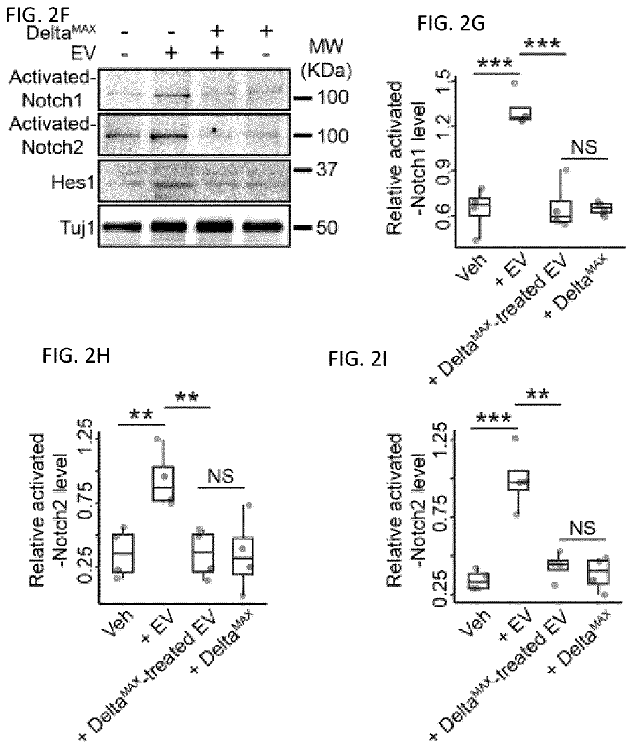

The canonical Notch signaling pathway begins at the cell surface by the binding of Notch_{ECD} to transmembrane ligands. Likewise, if Notch_{ECD} is present on the EV surface it could also bind Notch ligands expressed on the neuronal plasma membrane to activate signaling. To investigate how Notch is orientated within the EV membrane, purified EVs were incubated with proteinase K (PK) to digest peptide bonds accessible on the EV surface. Notch1_{ICD} and Notch2_{ICD} antibodies and WB analysis were used to determine which Notch protein domains are protected from digestion within the EV lumen. Notably, PK treatment reduced both Notch1 and Notch2 molecular weight by ˜10 kDa, consistent with the removal of the extracellular domain of Notch_{ICD} which anchors the Notch_{ECD} (FIG. 2A). Thus, Notch_{ECD} is present on the EV surface (FIG. 2B). By contrast, PK readily digested the multi-pass transmembrane EV protein CD81, but did not digest the EV luminal protein Sdcbp.

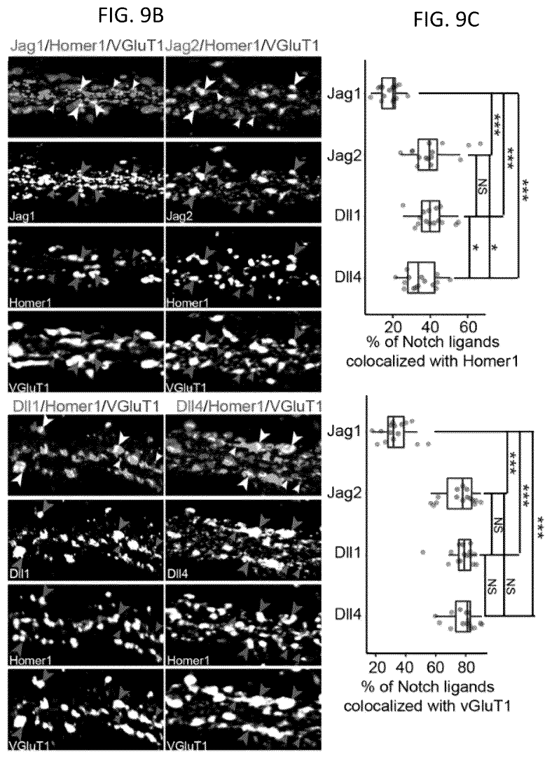

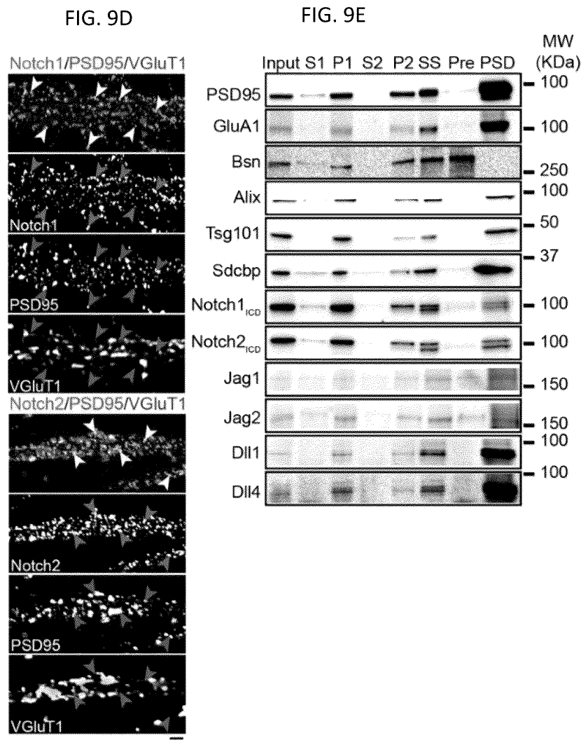

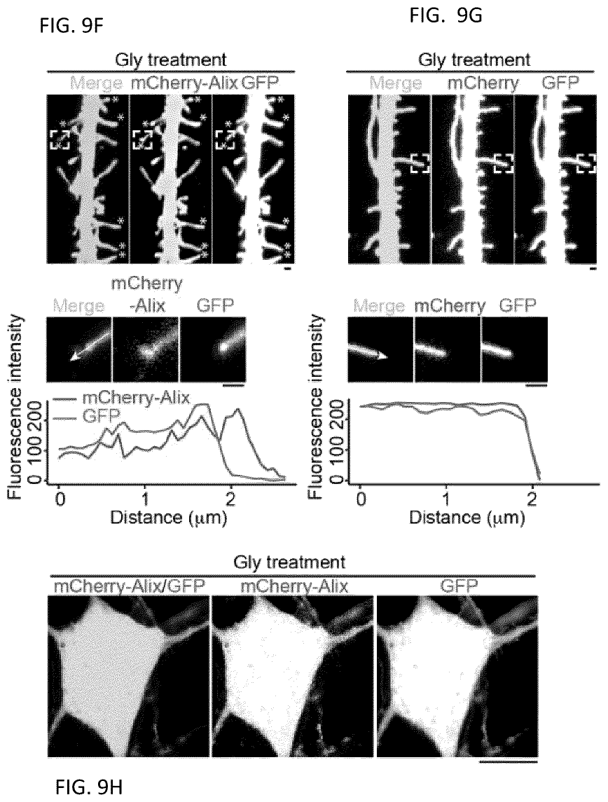

The binding of Notch_{ECD} to Notch ligands leads to the internalization of the ligand-receptor complex into the acceptor cell. Thus, it was hypothesized that EVs containing Notch_{ECD} on their surface may also be internalized. If so, internalized EVs should remain punctate after internalization and Notch ligands should colocalize with the internalized EVs. To test this hypothesis, neuronal EVs were labeled with the protein dye carboxyfluorescein succinimidyl ester (CFSE) to track internalized EVs. Visualization of the CFSE signals in neurons revealed that internalized EV proteins remain concentrated in puncta, which co-localize with Notch ligands, especially Jag 1, in neuronal soma and dendrites (FIGS. 2C & D). It was also found that Jag 1, Jag2, Dll1 and Dll4 are predominantly expressed by cultured rat hippocampal neurons (FIG. 9A). Immunocytochemistry (ICC) analysis indicates that Notch ligands partially colocalize with VGluT1 and Homer1, glutamatergic presynaptic and postsynaptic markers respectively (FIGS. 9B & C). Furthermore, Notch1 and Notch2 proteins also partially colocalize with VGluT1 and the excitatory postsynaptic marker PSD95 (FIG. 9D). However, the ability to discern pre- or postsynaptic colocalization is limited and even VGluT1 and Homer1 signals exhibit some colocalization (FIG. 9B). To further investigate the synaptic localization of Notch1 and Notch2 proteins, subcellular biochemical fractions of mouse cortex were examined by WB analysis (FIG. 9E). Notch 1_{ICD}, Notch 2_{ICD}, Dll1, and Dll4, are enriched within the postsynaptic density (PSD) and are nearly absent in the presynaptic (Pre) fractions. Notably, Jag1 and Jag2 are detected in both the Pre and PSD fractions. Thus, glutamatergic axon terminals and dendritic spines, which are hotspots of endocytosis, may represent active sites for EV internalization. The canonical EV proteins, tsg101, Alix, and Sdcbp, were also detected in the PSD fraction but not in the Pre fraction. This finding strongly suggests that dendritic spines are the primary sites for EV release. Live cell imaging was used to confirm that EVs were predominantly released from dendritic spines (FIG. 9F-H).

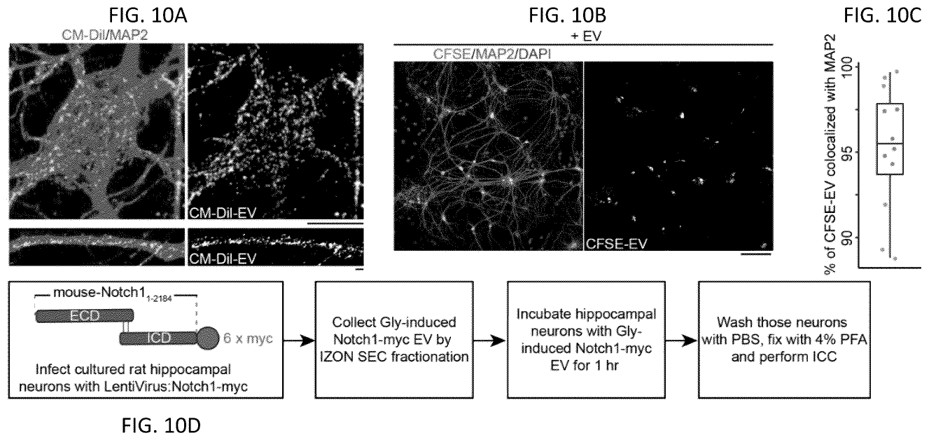

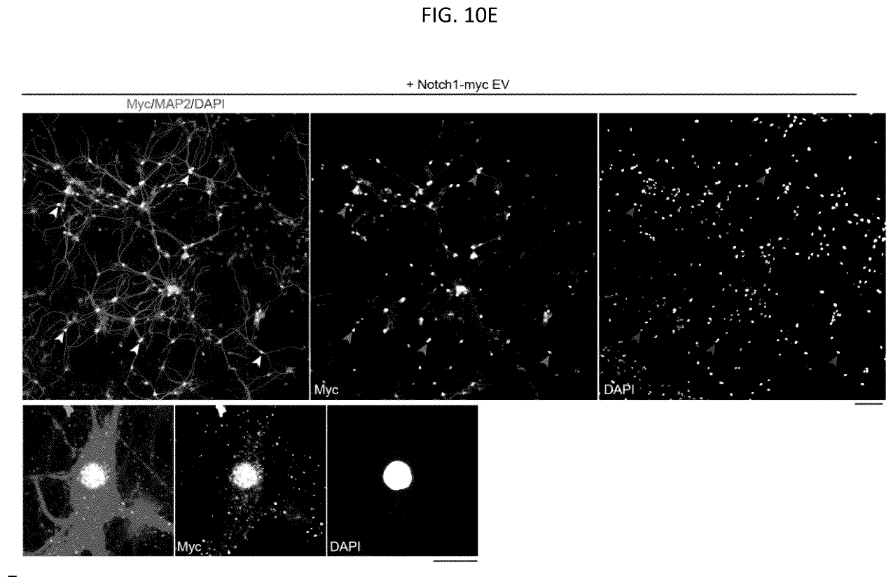

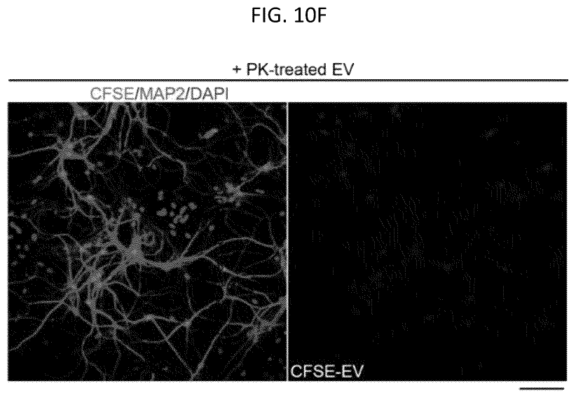

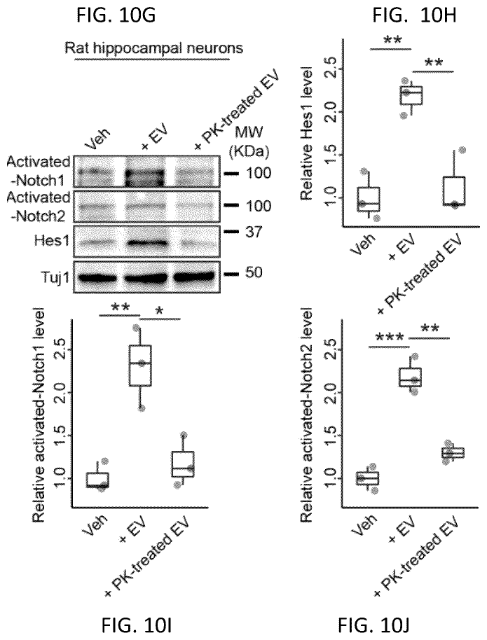

To investigate whether EVs directly fuse with the neuronal plasma membrane, which does not require endocytosis, EVs were labeled with the lipophilic dye CM-Dil. The CM-Dil labelled EVs were observed in a punctate pattern after internalization (FIG. 10A). This suggests that EV membrane integrity is maintained after internalization, rather than directly fusing with neuronal plasma membrane. Furthermore, CFSE labeled EVs are selectively internalized by MAP2 positive neurons (FIGS. 10B & C). To corroborate this finding, a Notch1 expression construct containing a C-terminal myc epitope tag²⁵ was packaged into lentiviruses. Neurons were infected with the lentivirus and glycine induced EVs were harvested. Next, additional non-infected neurons were treated with EVs containing C-terminal myc-tagged Notch1 (FIG. 10D). Consistently, the majority of myc signal was observed within MAP2-positive neurons, some myc signal was punctate, while other myc signal was highly enriched in the nucleus (FIG. 10E). This observation provides evidence that Notch_{ICD} from EVs can translocate into the nucleus of recipient neurons. Notably, EVs incubated with PK were not detected in neurons, which further supports a role for NECD-ligand trans interaction in mediating target specificity (FIGS. 2E & 10F).

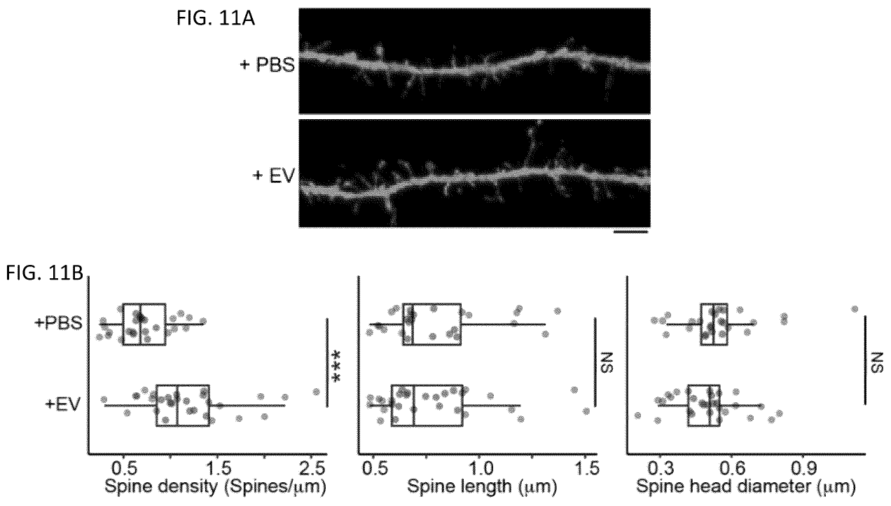

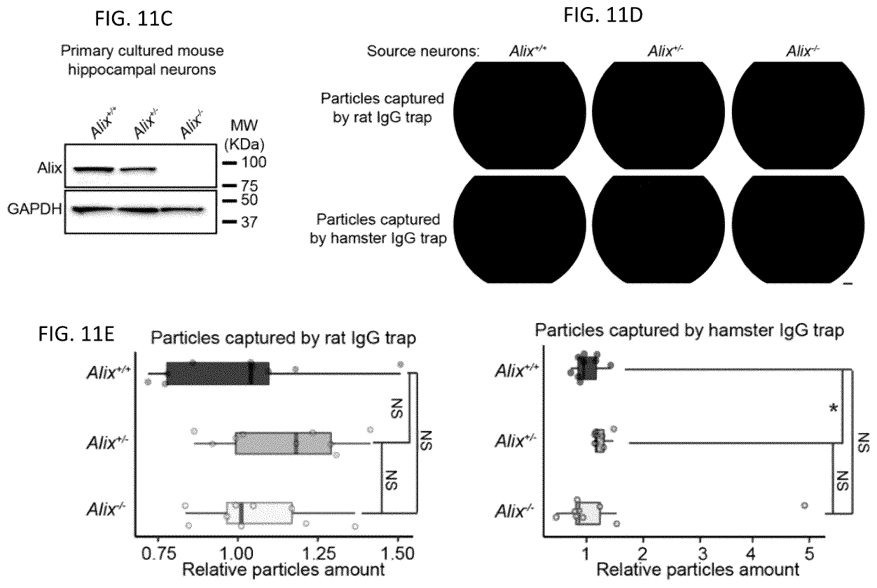

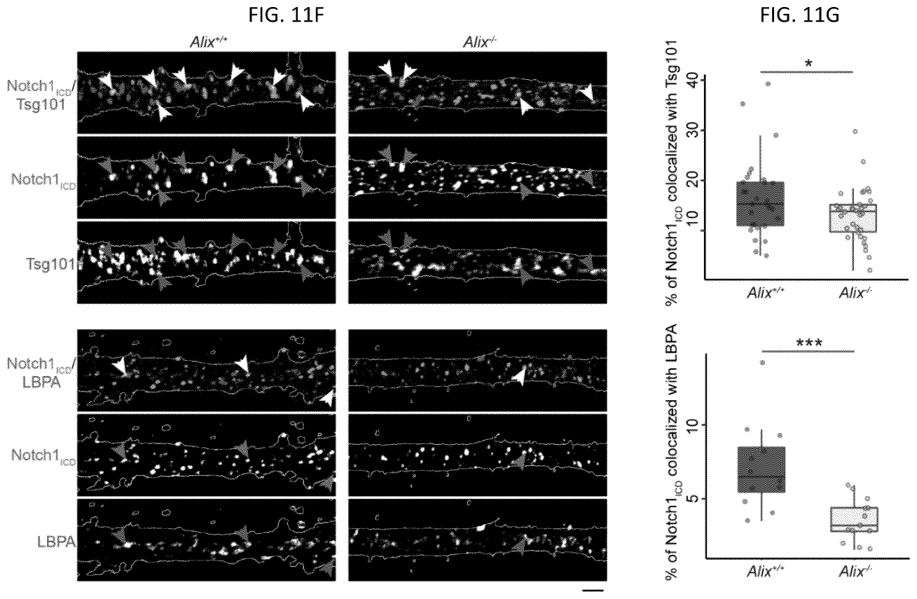

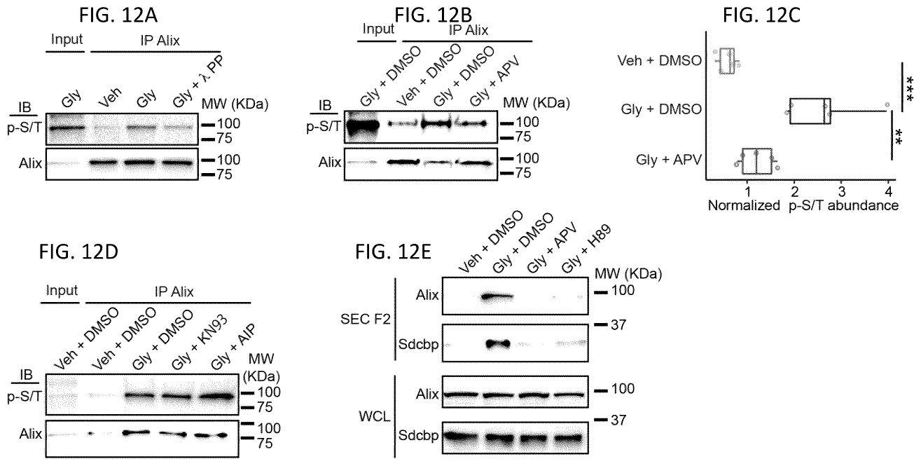

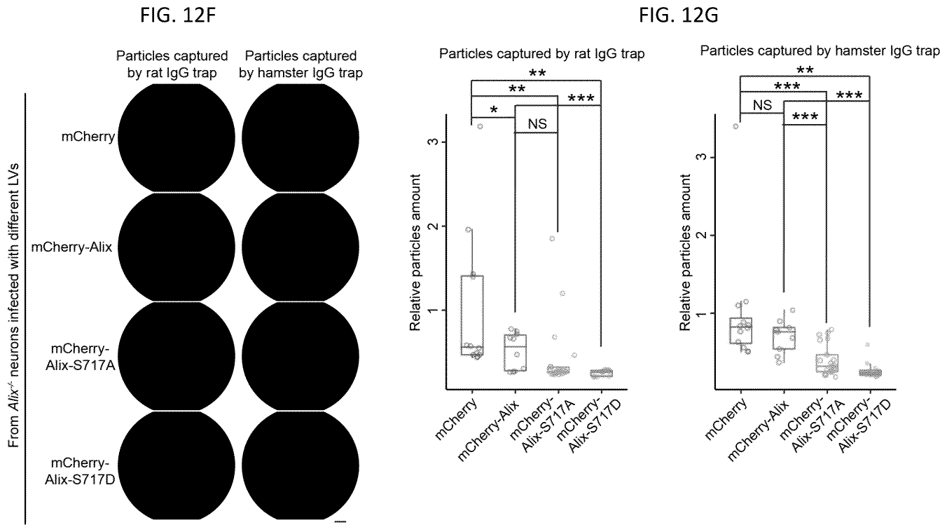

In light of the aforementioned observations, it was hypothesized that the internalization of EVs alone has the potential to activate the expression of Notch target genes, such as Hes1. To test whether Notch_{ICD} within EVs is functional, neuronal cultures were spiked with these EVs and the activation of the Notch pathway was examined by WB. In support of this hypothesis, the levels of activated-Notch1, -Notch2, and Hes1 proteins were all significantly elevated in neurons incubated with the EVs (FIGS. 2F-I and 10G-J). In contrast, EVs pretreated with PK failed to activate the Notch signaling pathway. To confirm that EV Notch proteins are responsible for activation of Notch signaling in recipient neurons, Notch EVs were incubated with the synthetic peptide Delta^{MAX26}. Delta^{MAX} that has very high binding affinity for murine Notch ECD and can be used to interfere with ligand-receptor biding. EVs preincubated with Delta^{MAX} were unable to initiate the Notch signaling pathway (FIG. 2F-I). To investigate if Notch EVs impact neuronal morphology, cultured neurons were incubated with the EVs for one hour. Significantly increased spine density was observed following culture (FIGS. 11A & B), suggesting EVs play a role in regulating the morphology of dendritic spines. Taken together, Notch_{ICD} within EVs can be further processed and drive Hes1 gene expression.

### Activation of Synaptic NMDARs Leads to Notch Target Gene Expression

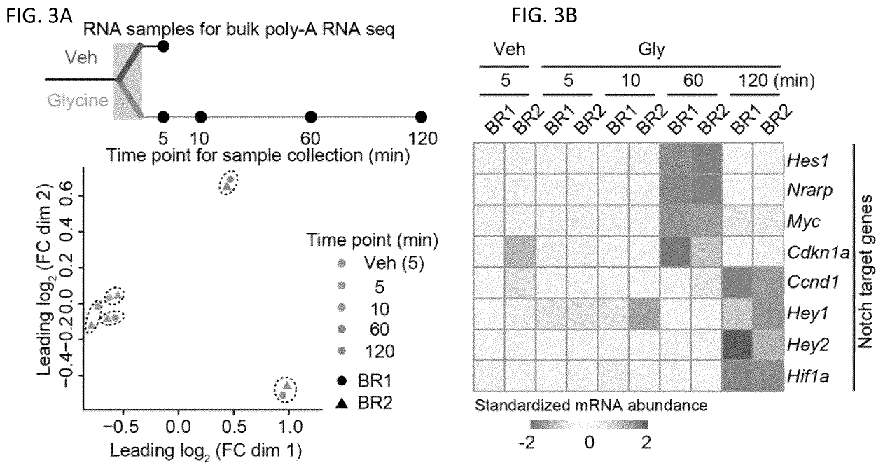

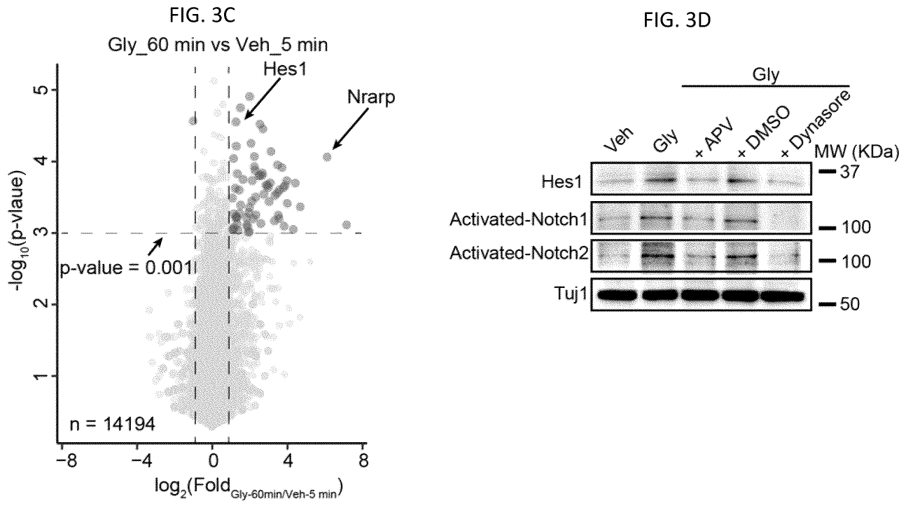

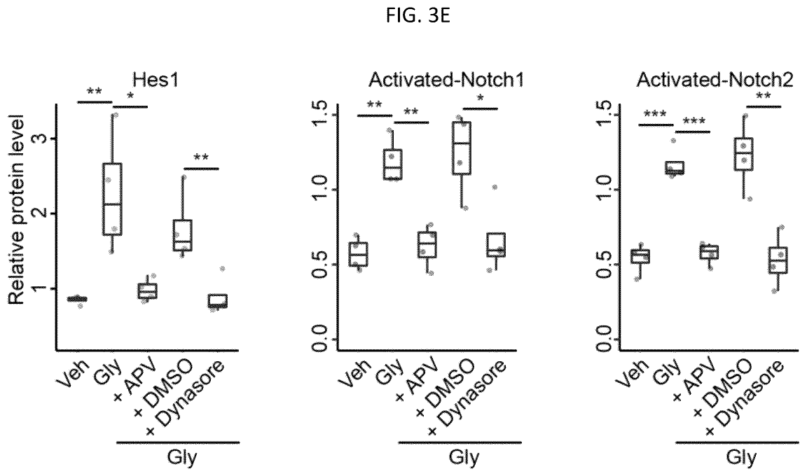

It was next investigated whether endogenous neuronal EV-mediated communication is sufficient to trigger Notch signaling in nearby neurons. Rat hippocampal neurons were stimulated with vehicle (Veh) or Mg²⁺-free glycine. In this experiment, instead of purifying EVs, neuronal RNA was harvested at multiple time points after treatment and bulk poly-A RNA sequencing was performed to examine changes in gene expression (FIG. 3A). Interestingly, synaptic NMDAR activation significantly induced expression of multiple Notch target genes (e.g., Hes1 and Nrarp) 60 mins after treatment (FIGS. 3B & C). Additionally, this stimulation also increased the level of activated-Notch1, -Notch 2, and Hes1 proteins (FIGS. 3D & E). This effect was inhibited by the NMDAR antagonist D-2-amino-5-phosphonovalerate (APV). Thus, at least in this system, activation of the Notch signaling pathway is NMDAR-dependent. Furthermore, inhibiting endocytosis with Dynasore blocked elevation of activated-Notch1, -Notch2, and Hes1 protein levels, suggesting internalization of EVs is required for Notch signaling pathway activation (FIGS. 3D & E). Altogether, this data indicate that endogenously released neuronal EVs are sufficient to activate the Notch signaling pathway.

### Synaptic NMDAR Activation of the EV Notch Signaling Pathway Requires Alix Expression

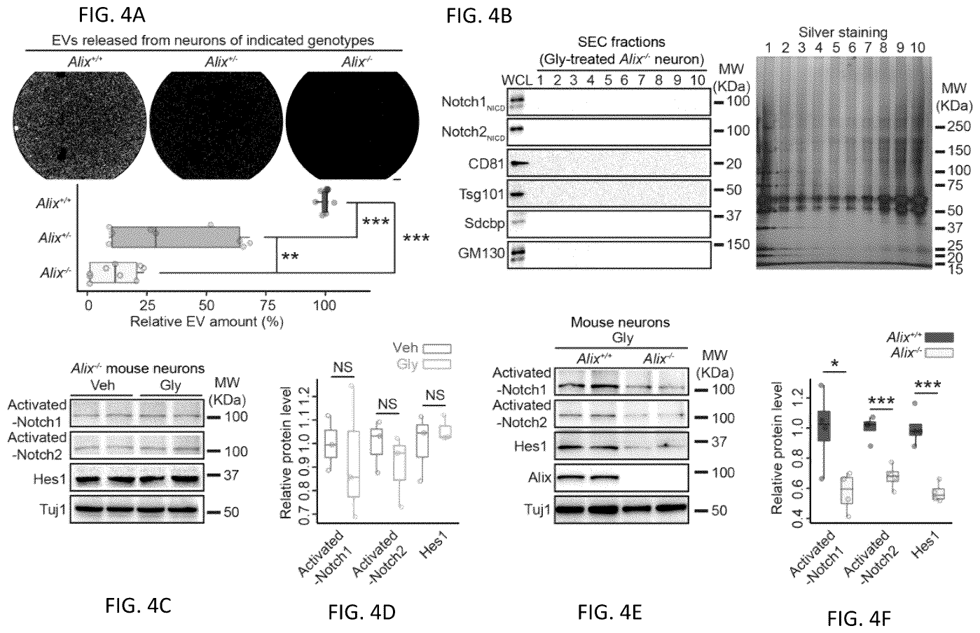

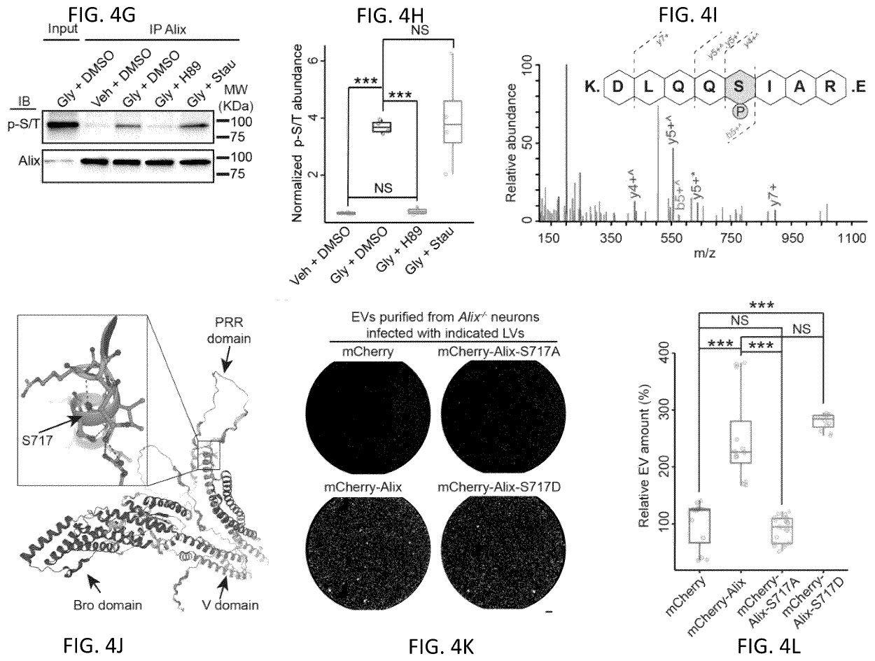

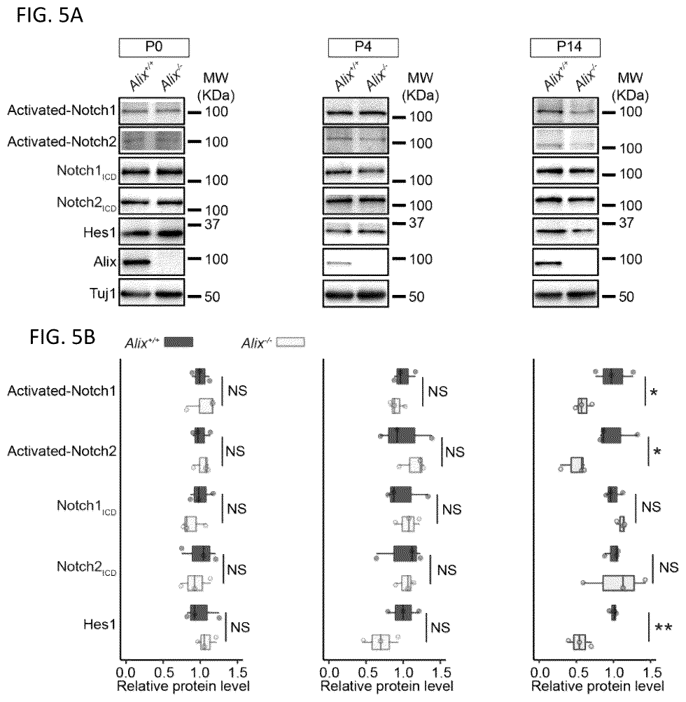

It was hypothesized that neurons with impaired EV biogenesis/release machinery are unable to activate the NMDAR-based EV signaling pathway. Alix (encoded by Pdcd6ip gene) functions in the ESCRT (endosomal sorting complex required for transport) pathway, is involved in EV biogenesis and release in multiple cell lines, thus it was also hypothesized that Alix may also play a role in neuronal EV release. Consistent with these hypotheses, activation of synaptic NMDARs induced significantly fewer EVs to be released from Alix^{+/-} and Alix^{-/-} neurons (FIG. 4A and FIG. 11C-E). No Notch proteins or known EV protein markers were detected in the SEC fractions by WB (FIG. 4B). These results show that Alix is required for synaptic NMDAR activation induced EV release. Moreover, as hypothesized, Mg²⁺-free glycine stimulation failed to increase activated-Notch1, -Notch2 or Hes1 protein levels in Alix^{-/-} neurons (FIG. 4C-F). Besides regulating EV release, Alix also contributes to protein cargo transport from early endosomes to multivesicular bodies (MVBs). Therefore, it was next examined whether the lack of Notch signaling pathway activation in Alix^{-/-} neurons is due to disruption of intracellular Notch protein trafficking. Alix deletion only slightly reduced Notch1 levels at Tsg101 and lysobisphosphatidic acid positive puncta (i.e., MVBs and late endosomes respectively) (FIGS. 11F & G). These mild changes suggest Notch protein trafficking is only marginally altered in Alix^{-/-} neurons. This alteration may be a mechanism underlying the abnormal embryonic brain development in Alix^{-/-} mice, which results from neuronal precursor apoptosis.

### Synaptic NMDAR Activation Triggers Alix Phosphorylation to Facilitate EV Release

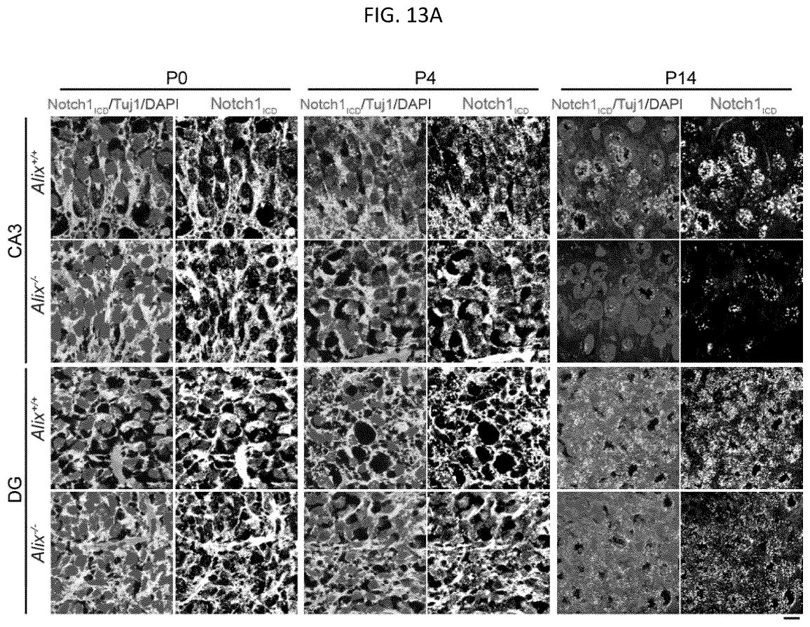

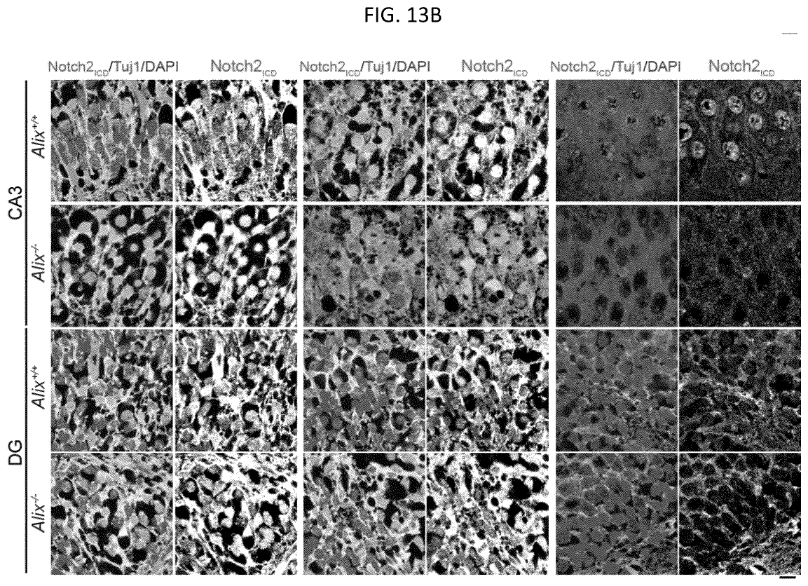

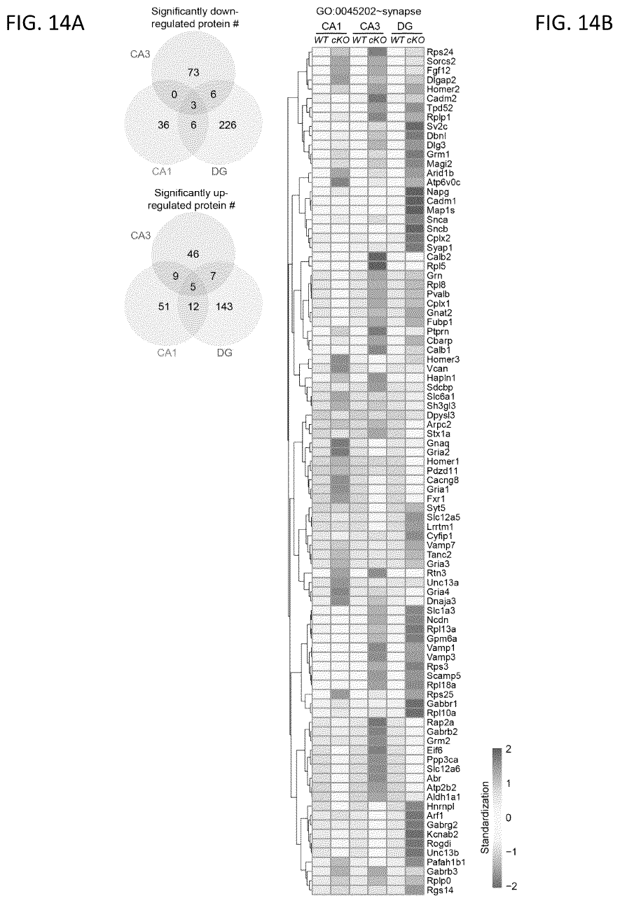

It was next investigated how activation of synaptic NMDARs promotes EV release and engages the Notch signaling pathway. Under basal conditions, Alix activity is suppressed by conformational autoinhibition through the interaction of proline-rich region (PRR) and BRO-domain containing protein (BRO) domains. In tumor cells, phosphorylation of Alix disrupts the PRR-BRO interaction and facilitates cytokinetic abscission and retroviral budding. Alix phosphorylation may also be required for EV release induced by synaptic activity. NMDAR activation led to Alix phosphorylation and was blocked by APV (FIG. 13A-C). Protein kinase A (PKA), protein kinase C (PKC), and Ca²⁺/calmodulin-dependent protein kinase II (Camk2) are engaged after activation of synaptic NMDARs. Thus, four kinase inhibitors were tested, and it was found only the PKA inhibitor H89 can significantly inhibit Alix phosphorylation (FIGS. 4G & H, 13D). Additionally, both APV and H89 can suppress NMDAR activity-induced EV release (FIG. 13E), which suggests both NMDAR and PKA are required for Mg²⁺-free glycine stimulation-induced EV release. To identify candidate Alix phosphorylation site(s) required for EV release, LC-MS/MS analysis of neurons treated with Mg²⁺-free glycine or vehicle solutions was performed. Alix is phosphorylated at serine 717 (S717) (FIG. 4I). No phosphorylated Alix peptides were identified in vehicle-treated neurons. S717 localizes to the junction between the V-domain and the PRR domain (FIG. 4J), which is evolutionarily conserved among animals. To test whether phosphorylation of S717 contributes to NMDAR activity-induced EV release, the serine was mutated to aspartic acid (S717D) to mimic constitutive phosphorylation. A phosphorylation-dead mutant was constructed by replacing the serine with alanine (S717A). These two constructs were packaged into lentiviruses and used to infect Alix^{-/-} hippocampal neurons. Overexpression of mCherry-Alix or mCherry-Alix-S717D, but not mCherry or mCherry-Alix-S717A, successfully rescued NMDAR activity-induced EV release (FIGS. 4 K & L, 13F & G). Altogether, this data demonstrates that Alix phosphorylation plays a role in synaptic NMDAR activation-induced EV release.

### Notch Signaling is Reduced in Adult Alix^{-/-} Mouse Hippocampus

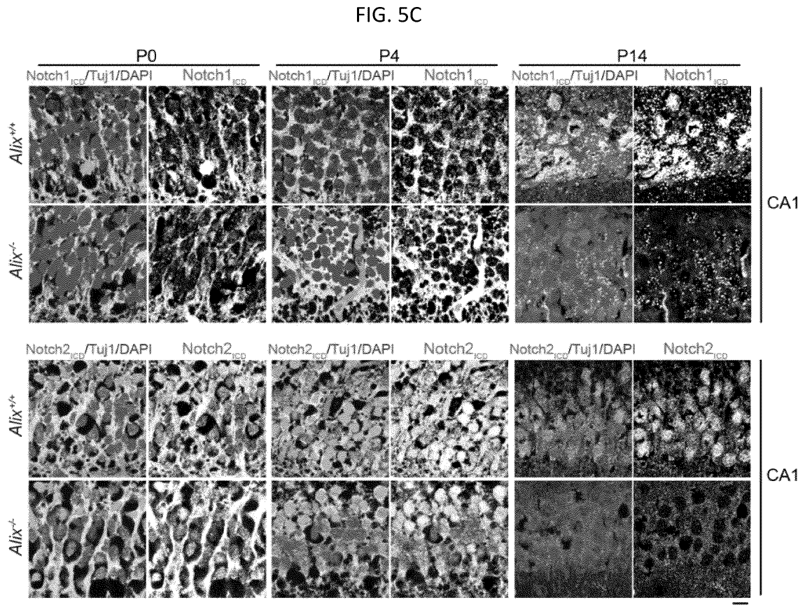

Alix is a multifunctional protein that is associated with the ESCRT machinery and is involved in several membrane centric processes including membrane repair, MVB and exosome biogenesis, and neuronal activity-dependent bulk endocytosis. Since bulk endocytosis facilitates EV uptake, Alix^{-/-} neurons may also have hampered EV internalization. Therefore, Alix^{-/-} mice have severely hampered EV processes, and it was hypothesized that the EV Notch signaling pathway is also impaired. During the very early stages of postnatal brain development (i.e., P0-P4), there is nearly no excitatory synaptic transmission in the brain. Consistently the level of Notch1_{ICD}, Notch2_{ICD}, activated-Notch1, -Notch2 and Hes1 proteins are very similar in Alix^{+/+} and Alix^{-/-} hippocampal extracts (FIGS. 5A & B). In contrast, at P14, when excitatory synaptic transmission is established, the levels of activated-Notch1, -Notch2 and Hes1 proteins in Alix^{+/+} hippocampus are significantly higher than in Alix^{-/-} hippocampus (FIGS. 5A & B). However, the levels of Notch1_{ICD} and Notch2_{ICD} remain similar.

Notch_{ICD} translocates to the nucleus to induce expression of Notch target genes. Thus, the degree of Notch signaling pathway activation is correlated with the abundance of nuclear Notch_{ICD}. At P0 and P4, the expression patterns of Notch1_{ICD} and Notch2_{ICD} are very similar in Alix^{+/+} and Alix^{-/-} hippocampi (FIGS. 5C & S6). However, at P14, there is less nuclear Notch1_{ICD} and Notch2_{ICD} in Alix^{-/-} hippocampal CA1 and CA3 regions compared to Alix^{+/+} hippocampus. There was also less Notch1_{ICD} in P14 Alix^{-/-} dentate gyrus (DG). Notably, the expression patterns of Notch2_{ICD} are very similar in Alix^{+/+} and Alix^{-/-} DG (FIGS. 5C & S6B). Therefore, this data strongly suggest that Notch pathway activation is associated with the developmental onset of excitatory synaptic transmission and that impaired neuronal EV-based signaling decreases Notch pathway activation in the mouse hippocampus.

### Alix is Required for Activation of the Notch Signaling Pathway in the Adult Mouse Hippocampus

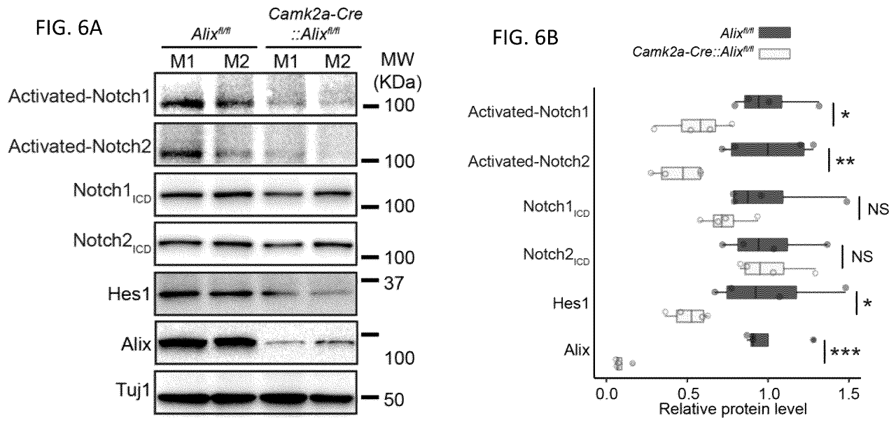

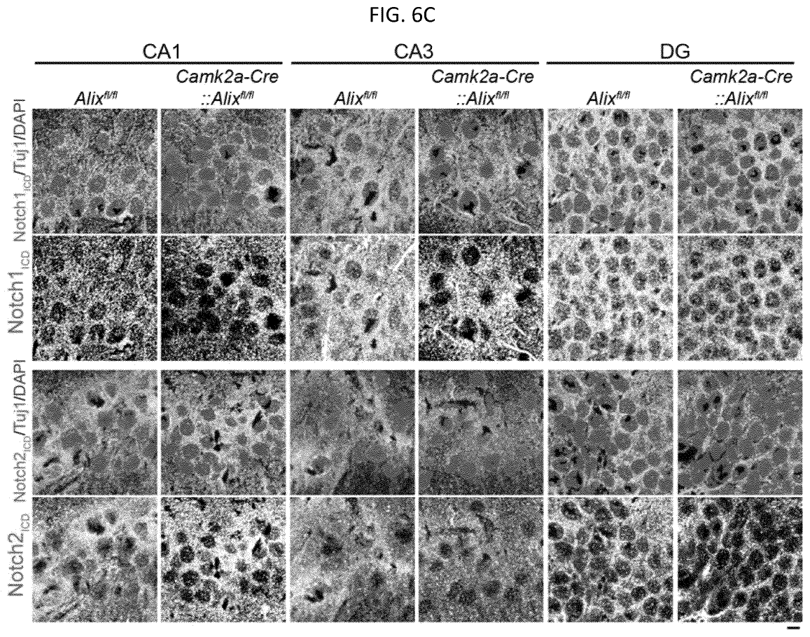

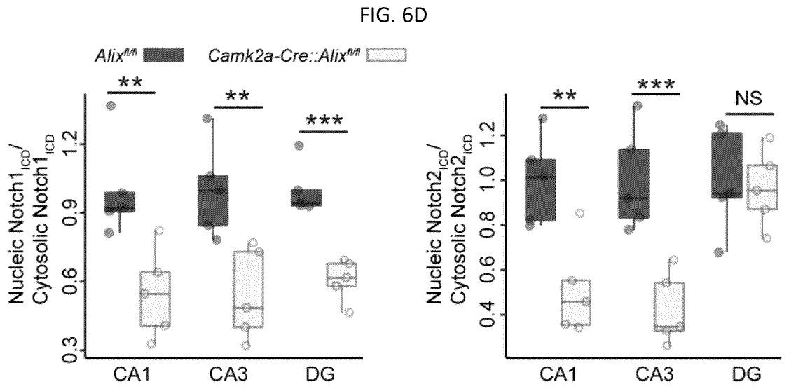

Alix is involved in brain development and Alix^{-/-} mouse brains are significantly smaller compared to wild type. To avoid the defects in embryonic development, Alix-floxed (Alix^{fl/fl}) mice were crossed with Camk2a-cre mice to obtain Camk2a-cre:: Alix^{fl/fl}. Camk2a-cre is highly expressed in pyramidal neurons of adult mouse hippocampus and turns on around P30. At P60, Alix protein levels in Camk2a-cre:: Alix^{fl/fl} hippocampus were reduced to ˜5% of the level in Alix^{fl/fl} hippocampus (FIGS. 6A & B). The level of activated-Notch1, -Notch2 and Hes1 proteins were also significantly reduced in Camk2a-cre:: Alix^{fl/fl} hippocampus compared to Alix^{fl/fl} hippocampus. However, Notch1_{ICD} and Notch2_{ICD} protein levels remained similar, suggesting the reduction is not due to downregulation of Notch protein expression.

The expression patterns of Notch1_{ICD} and Notch2_{ICD} in Alix^{fl/fl} and Camk2a-cre:: Alix^{fl/fl} hippocampi (FIGS. 6C & D) were further examined. Compared to Alix^{fl/fl} hippocampus, the amount of nuclear Notch1_{ICD} was significantly reduced in Alix conditional knockout (cKO) CA1, CA3 and DG regions. By contrast, conditional knockout of Alix significantly reduced the amount of nuclear Notch2_{ICD} in CA1 and CA3, but not DG. This region-specific alteration of Notch1_{ICD} and Notch2_{ICD} expression patterns suggests Notch1 and Notch2 may exist in separate populations of neuronal EVs and are released by different neurons. Collectively, these data indicate that hampered neuronal EV release limits activation of the Notch pathway in the adult brain.

### Conditional Deletion of Alix in the Adult Mouse Brain Leads to Hippocampal Subregion-Specific Proteomic Changes

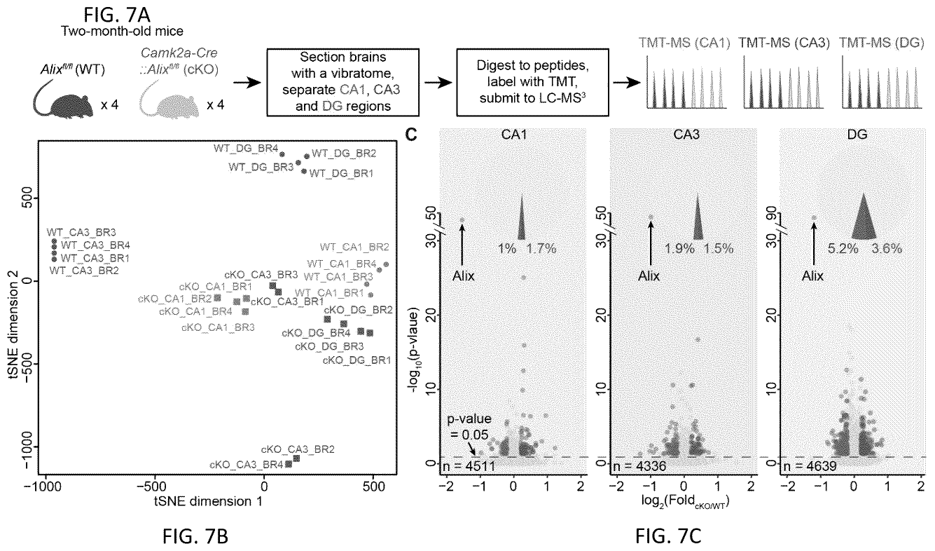

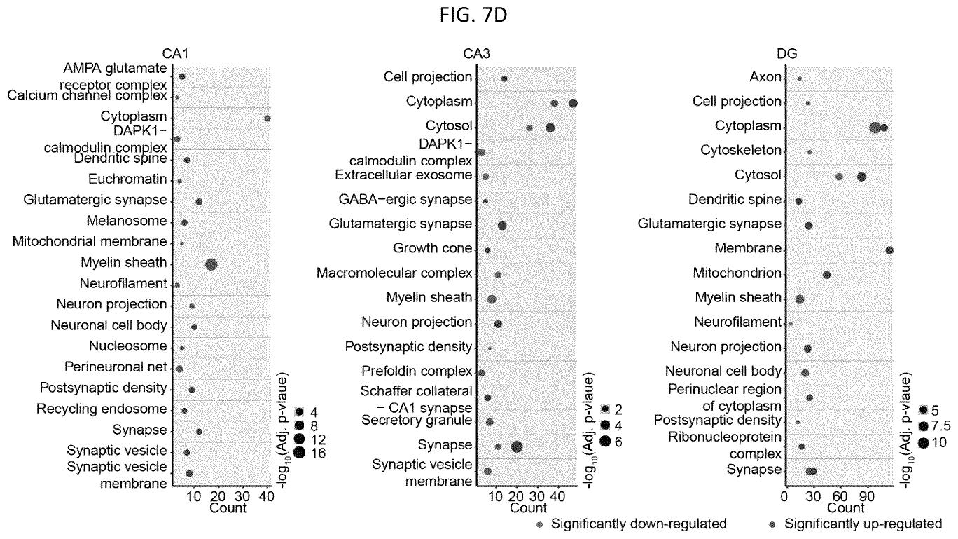

The Notch signaling pathway plays important roles in synaptic function. To investigate the consequences of reduced Notch signaling activity in the mouse hippocampus, Tandem Mass Tag (TMT)-based proteomic analysis was used to quantitatively compare the Alix^{fl/fl} and Camk2a-cre:: Alix^{fl/fl} hippocampal CA1, CA3 and DG proteomes respectively (FIGS. 7 & 14). The conditional deletion of Alix leads to distinct proteomic changes in CA1, CA3, and DG (FIGS. 7B & C). This finding aligns with the region-specific changes in Notch 1_{ICD} and Notch 2_{ICD} levels within the hippocampus of Camk2a-cre:: Alix^{fl/fl} brain (FIGS. 6C & D). Notably, the DG proteome showed the most robust changes (FIGS. 7C & 14A), which suggests that EV-mediated Notch1 activation may play an important role in DG. The Database for Annotation, Visualization and Integrated Discovery analysis of the downregulated proteins revealed a significant enrichment of the term “glutamatergic synapses” in the datasets from all three regions (top 2 in CA1, top 3 in CA3, top 7 in DG, FIG. 7D). This finding is consistent with a recent study showing that Alix^{-/-} hippocampal neurons have shorter postsynaptic densities and smaller spine head size compared to WT controls 44. This data indicates that EV-mediated neural communication is required for normal glutamatergic synaptic protein expression in the hippocampus.

### DISCUSSION

Herein it is demonstrated that synaptic NMDARs trigger hippocampal neurons to release Notch 1 and Notch2 proteins in EVs. These EVs facilitate short range, irreversible, cell type-specific Notch signaling selectively in neurons. This EV-based Notch signaling pathway requires the ESCRT-associated protein Alix, and adult Alix cKO mice have reduced hippocampal Notch signaling. Thus, EVs mediate neuron-to-neuron communication, in which the Notch1 and Notch2 proteins play a neurotransmitter-like role, and the Notch ligands are the receptors for EV signaling.

These results indicate Notch receptor-ligand binding facilitates EV capture on the neuronal plasma membrane and subsequent endocytosis. In general most EVs have intact membranes after internalization (FIGS. 2C & E, and 10A). Endocytosis occurs at many locations spread across the neuronal plasma membrane but glutamatergic presynaptic sites are particularly active sites of endocytosis. Presynaptic Jag1 and Jag2 may regulate the EV uptake in axon terminals. Interestingly, a small portion of the Jag1 and Jag2 pool was also detected in the PSD fraction (FIGS. 9B, C & E). In contrast, synaptic Dll1 and Dll4 are exclusively detected within the postsynaptic compartment. Dendrites and spines are also endocytosis hot spots. Therefore, postsynaptic Jag 1, Jag2, Dll1 and Dll4 may contribute to the EV uptake in dendritic spines. After internalization, EVs are transported to the soma through retrograde trafficking. Once activated, Notch_{ICD} is proteolytically processed, enters the nucleus and initiates Notch target gene expression. However, the mechanisms governing the translocation of EV-associated Notch receptors into the nucleus remains unclear.

These findings linking the Notch signaling pathway to EVs provide a new perspective on more than one hundred years of research on this evolutionarily conserved cell-to-cell communication pathway. For example, in CNS neurons, Notch1 and Notch2 have been found to be expressed at dendritic spines, and Jag1 are mainly present at presynaptic sites. Thus, in this arrangement, binding of the postsynaptic receptors and the presynaptic ligands in trans would result in endocytosis of the Notch receptor-ligand complex and constitutive activation of the Notch signaling pathway in the presynaptic neurons. However, in addition to the plasma membrane and cycling endosomes, Notch proteins also localize to intralumenal vesicles (ILVs) within MVBs. In hippocampal neurons, MVBs are mainly distributed in cell bodies and dendrites, suggesting that the postsynaptic Notch proteins may be primarily localized to dendritic ILVs rather than to the dendritic spine plasma membrane. Notably, MVBs are sites of EV biogenesis and storage, including exosomes, and Notch proteins have been observed in MVBs that were previously thought to be en route to lysosomal degradation. However, the results presented herein show that Notch-containing dendritic ILVs are also be secreted as EVs in an Alix-dependent manner in the mammalian neurons (FIG. 4). Other parallel mechanisms exist for EV biosynthesis and release, including non-canonical ESCRT-dependent pathways and ESCRT-independent route. Notably, Alix is not essential for these EV biosynthesis or release pathways.

In the adult brain, Notch signaling activity contributes to synaptic plasticity, learning, and memory. Furthermore, genetic inactivation of the Notch target gene Hes1 expression in excitatory neurons resulted in abnormal fear and anxiety-related behavior in adult mice. In the context of the findings herein, EV-based Notch signaling may underlie these observations in the adult brain. In vitro EVs were found to require at least one hour to induce gene expression after stimulation of synaptic NMDARs (FIG. 3B). Therefore, EVs are unlikely to regulate short term synaptic plasticity and are more likely to play a key role in regulating long-term synaptic processes such as late-phase long-term potentiation and memory consolidation. Notably, since EVs also commonly contain nucleic acids (i.e., miRNA and mRNA), EV-based neuronal communication likely to also regulate gene expression beyond Notch target genes. In summary, the data presented herein demonstrate that EVs facilitate cell type specific communication between neurons through the Notch ligand-receptor system.

### Key Resources Table

| | | |
|---|---|---|
| REAGENT or | | |
| RESOURCE | SOURCE | IDENTIFIER |

Antibodies

| | | |
|---|---|---|
| chicken anti-MAP2 | Millipore Sigma | Cat#AB5543, RRID: AB_571049 |
| mouse anti-Jag1 | Santa Cruz | Cat# sc-390177, RRID: AB_2892141 |
| | Biotechnology | |
| goat anti-Jag2 | Thermo Fisher | Cat# PA5-47188, RRID: AB_2576459 |
| | Scientific | |
| rabbit anti-Jag2 | Cell Signaling | Cat# 2210, RRID: AB_823553 |
| | Technology | |
| goat anti-Dll1 | Abcam | Cat# ab85346, RRID: AB_1860332 |
| rabbit anti-Dll1 | Cell Signaling | Cat# 2588, RRID: AB_2292961 |
| | Technology | |
| goat anti-Dll4 | Thermo Fisher | Cat# PA5-46974, RRID: AB_2577158 |
| | Scientific | |
| rabbit anti-Dll4 | Cell Signaling | Cat# 96406, RRID: AB_2800263 |
| | Technology | |
| guinea pig anti-VGluT1 | Millipore Sigma | Cat# AB5905, RRID: AB_2301751 |
| rabbit anti-Homer1 | Synaptic Systems | Cat# 160 003, RRID: AB_887730 |
| mouse anti-Tsg101 | Santa Cruz | Cat# sc-7964, RRID: AB_671392 |
| | Biotechnology | |
| rabbit anti-Notch1_{ICD} | Abcam | Cat# ab52627, RRID: AB_881725 |
| rabbit anti- | Abcam | Cat# ab52301, RRID: AB_881726 |
| activated Notch1 | | |
| rabbit anti- | Cell Signaling | Cat# 5732, RRID: AB_10693319 |
| Notch2_{ICD} | Technology | |
| rabbit anti- | MyBioSource | Cat# MBS9410510 |
| activated Notch2 | | |
| mouse anti-PSD95 | Thermo Fisher | Cat# MA1-046, RRID: AB_2092361 |
| | Scientific | |
| rabbit anti- | Abcam | Cat# ab76108, RRID: AB_1310621 |
| PSD95(pS295) | | |
| mouse anti-Alix | Cell Signaling | Cat# 2171, RRID: AB_2299455 |
| | Technology | |
| rabbit anti-Alix | Cell Signaling | Cat# 18269, RRID: AB_2798796 |
| | Technology | |
| mouse anti-CD81 | Santa Cruz | Cat# sc-166029, RRID: AB_2275892 |
| | Biotechnology | |
| rabbit anti-Sdcbp | Abcam | Cat# ab19903, RRID: AB_445200 |
| rabbit anti-Sdcbp | Cell Signaling | Cat # 27964 |
| | Technology | |
| mouse anti-LBPA | Millipore Sigma | Cat # MABT837 |
| rabbit anti-GM130 | Abcam | Cat# ab52649, RRID: AB_880266 |
| rabbit anti-Hes1 | Cell Signaling | Cat# 11988, RRID: AB_2728766 |
| | Technology | |
| rabbit anti-Hes1 | Abcam | Cat# ab71559, RRID: AB_1209570 |
| chicken anti-Tuj1 | Novus | Cat# NB100-1612, RRID: AB_10000548 |
| | Biologicals | |
| rabbit anti-p-S/T | Cell Signaling | Cat# 9624, RRID: AB_331817 |
| | Technology | |
| rabbit anti-GAPDH | Cell Signaling | Cat# 2118, RRID: AB_561053 |
| | Technology | |
| chicken anti-GFP | Abcam | Cat# ab13970, RRID: AB_300798 |
| mouse anti-myc | Santa Cruz | Cat# sc-40, RRID: AB_627268 |
| | Biotechnology | |
| rabbit anti-GluA1 | EMD Millipore | Cat# PC246, RRID: AB_564636 |
| guinea pig anti- | Synaptic | Cat# 141 318, RRID: AB_2927388 |
| Bassoon | Systems | |
| Goat anti-Mouse | Thermo | Cat# A-11029, RRID: AB_2534088 |
| IgG (H + L) Highly | Scientific | |
| Cross-Adsorbed | | |
| Secondary | | |
| Antibody, Alexa | | |
| Fluor 488 | | |
| Donkey anti-Goat | Thermo | Cat# A-11057, RRID: AB_2534104 |
| IgG (H + L) Cross- | Scientific | |
| Adsorbed Secondary | | |
| Antibody, Alexa | | |
| Fluor 568 | | |
| Donkey anti- | Thermo | Cat# A10037, RRID: AB_2534013 |
| Mouse IgG (H + L) | Scientific | |
| Highly Cross- | | |
| Adsorbed Secondary | | |
| Antibody, Alexa | | |
| Fluor 568 | | |
| Goat anti-Rabbit | Thermo | Cat# A-11011 (also A11011), RRID: AB_143157 |
| IgG (H + L) Cross- | Scientific | |
| Adsorbed Secondary | | |
| Antibody, Alexa | | |
| Fluor 568 | | |
| Donkey anti-Goat | Thermo | Cat# A-11055 (also A11055), RRID: AB_2534102 |
| IgG (H + L) Cross- | Scientific | |
| Adsorbed Secondary | | |
| Antibody, Alexa | | |
| Fluor 488 | | |
| Goat anti-Chicken | Thermo | Cat# A-21437, RRID: AB_2535858 |
| IgY (H + L) | Scientific | |
| Secondary | | |
| Antibody, Alexa | | |
| Fluor 555 | | |
| Goat anti-Rabbit | Thermo | Cat# A-11034 (also A11034), RRID: AB_2576217 |
| IgG (H + L) Highly | Scientific | |
| Cross-Adsorbed | | |
| Secondary | | |
| Antibody, Alexa | | |
| Fluor 488 | | |
| Goat Anti-Guinea | Abcam | Cat# ab150187, RRID: AB_2827756 |
| pig IgG H&L | | |
| (Alexa Fluor 647) | | |
| Goat anti-Rabbit | Thermo | Cat# 32260, RRID: AB_1965959 |
| IgG (H + L) Poly- | Scientific | |
| HRP Secondary | | |
| Antibody, HRP | | |
| Goat anti-Mouse | Thermo | Cat# 32230, RRID: AB_1965958 |
| IgG (H + L) Poly- | Scientific | |
| HRP Secondary | | |
| Antibody, HRP | | |
| Goat anti-Chicken | Abcam | Cat# ab97135, RRID: AB_10680105 |
| IgY H&L (HRP) | | |
| secondary antibody | | |
| IgG(H + L) Goat | Fisher Scientific | Cat# OB6090-05 |
| anti-Guinea Pig, | | |
| HRP, Polyclonal, | | |
| Southern Biotech | | |

Bacterial and virus strains

| | | |
|---|---|---|
| pz196-mCherry | Dr. Evangelos | N/A |
| | Kiskinis lab | |
| pz196-mCherry- | Dr. Evangelos | N/A |
| Alix | Kiskinis lab | |
| pz196-mCherry- | Dr. Evangelos | N/A |
| Alix-S717A | Kiskinis lab | |
| pz196-mCherry- | Dr. Evangelos | N/A |
| Alix-S717D | Kiskinis lab | |
| pz196-Notch1- | Dr. Evangelos | N/A |
| myc | Kiskinis lab | |

Chemicals, peptides, and recombinant proteins

| | | |
|---|---|---|
| poly-D-lysine | Fisher Scientific | Cat# CB-40210 |
| laminin | Fisher Scientific | Cat# 23-017-015 |
| SM1 | STEMCELL | Cat# 05711 |
| | Technologies | |
| 5-fluoro-2- | Sigma-Aldrich | Cat# F0503 |
| deoxyuridine | | |
| uridine | Sigma-Aldrich | Cat# U6381 |
| NaCl | Sigma-Aldrich | Cat# S7653 |
| KCl | Sigma-Aldrich | Cat# P9333 |
| NaH₂PO₄ | Sigma-Aldrich | Cat# 71505 |
| NaHCO₃ | Sigma-Aldrich | Cat# S6014 |
| glucose | Sigma-Aldrich | Cat# G7021 |
| CaCl₂ | Sigma-Aldrich | Cat# C1016 |
| MgCl₂ | Sigma-Aldrich | Cat# M8266 |
| CsF | Sigma-Aldrich | Cat# 198323 |
| CsCl | Sigma-Aldrich | Cat# 289329 |
| HEPES | Sigma-Aldrich | Cat# H3375 |
| EGTA | DOT Scientific Inc | Cat# DSE57060 |
| Mg-ATP | Sigma-Aldrich | Cat# A9187 |
| QX-314 | Sigma-Aldrich | Cat# 552233 |
| TEA-Cl | Sigma-Aldrich | Cat# 86614 |
| 4-AP | Sigma-Aldrich | Cat# 275875 |
| CsOH | Sigma-Aldrich | Cat# C8518 |
| TTX | Fisher Scientific | Cat# NC0738940 |
| picrotoxin | Sigma-Aldrich | Cat# P1675 |
| bicuculline | Sigma-Aldrich | Cat# 14340 |
| glycine | Sigma-Aldrich | Cat# G5417 |
| strychnine | Sigma-Aldrich | Cat# S0532 |
| CFSE | Thermo Fisher | Cat# C34554 |
| | Scientific | |
| CM-Dil | Thermo Fisher | Cat# C7000 |
| | Scientific | |
| DMSO | Sigma-Aldrich | Cat# D2650 |
| Proteinase k | Fisher Scientific | Cat# NC0547027 |
| PFA | Fisher Scientific | Cat# AAA1131336 |
| Triton X-100 | Sigma-Aldrich | Cat# X100 |
| DAPI | Sigma-Aldrich | Cat# D9542 |
| Fluoromount-G | Southern Biotech | Cat# 0100-01 |
| dynasore | Sigma-Aldrich | Cat# SML0340 |
| H89 | Sigma-Aldrich | Cat# B1427 |
| staurosporine | Tocris | Cat# 1285 |
| APV | Sigma-Aldrich | Cat# A5282 |
| KN93 | Sigma-Aldrich | Cat# K1385 |
| AIP | Anaspec | Cat# AS-64929 |
| Trizma base | Sigma-Aldrich | Cat# RDD008 |
| SDS | DOT Scientific Inc | Cat# DSL22040 |
| EDTA | DOT Scientific Inc | Cat# DS170184 |
| sodium deoxycholate | Sigma-Aldrich | Cat# D6750 |
| protease inhibitor | Thermo Fisher | Cat# 78443 |
| cocktail | Scientific | |
| phosphatase | Thermo Fisher | Cat# 78420 |
| inhibitor | Scientific | |
| BSA | Jackson | Cat# 001-000-162 |
| | immunoResearch | |
| | laboratories, | |
| Tween 20 | DOT Scientific Inc | Cat# DSP20370-4 |
| Tris-Cl | Sigma-Aldrich | Cat# T5941 |
| NaF | Sigma-Aldrich | Cat# S7920 |
| β-glycerophosphate | Sigma-Aldrich | Cat# G9422 |
| sodium orthovanadate | Sigma-Aldrich | Cat# 450243 |
| N-ethylmaleimide | Sigma-Aldrich | Cat# E3876 |
| Protein G Sepharose | Thermo Fisher | Cat# 45-000-116 |
| 4 Fast Flow | | |
| IgG | Sigma-Aldrich | Cat# 15381 |
| dithiothreitol | DOT Scientific Inc | Cat# DSD11000 |
| MnCl₂ | Sigma-Aldrich | Cat# M8266 |
| lambda | New England | Cat# P0753 |
| phosphatase | Biolabs | |
| embedding medium | Sakura | Cat# 4583 |
| ProLong Diamond | Thermo Fisher | Cat# P36970 |
| Antifade Mountant | Scientific | |
| Trichloroacetic acid | Sigma-Aldrich | Cat# T0699 |
| urea | Thermo Fisher | Cat# 29700 |
| | Scientific | |
| NH₄HCO₃ | Fluka | Cat# 09830 |
| iodoacetamide | Sigma-Aldrich | Cat# I1149 |
| Lys-C | Thermo Fisher | Cat# 90307_3668048707 |
| | Scientific | |
| Trypsin | Promega | Cat# V5280 |
| trifluoroacetic acid | Fisher Scientific | Cat# O4902-100 |
| TMT | Thermo Fisher | Cat# 44520 |
| | Scientific | |
| acetonitrile | Fisher Scientific | Cat# A955-212 |
| hydroxylamine | Sigma-Aldrich | Cat# 467804 |
| Delta^{MAX} | GenScript & Dr. | Custom synthesis |
| | Vincent Luca lab | |

Experimental models: Organisms/strains

| | | |
|---|---|---|
| Wistar rat | Charles River | 273 |
| Alix^{+/-} mice | Dr. Alessandra | N/A |
| | d'Azzo lab | |
| Alix^{fl/fl} mice | Dr. Rémy Sadoul | N/A |
| | and Dr. Frank | |
| | Kirchhoff lab | |
| Camk2a-Cre mice | The Jackson | 005359 |
| | Laboratory | |

Recombinant DNA

| | | |
|---|---|---|
| mCherry-Alix | Addgene | Cat# 21504 |
| GFP | Dr. Peter Penzes | N/A |
| | lab | |
| pz196 | Dr. Evangelos | N/A |
| | Kiskinis lab | |
| Notch1-myc | Addgene | Cat# 41728 |

Software and algorithms

| | | |
|---|---|---|
| Spyder | MIT | spyder-ide.org/ |
| RStudio | Posit PBC | posit.co/products/open-source/rstudio/ |
| DAVID | NIH | david.ncifcrf.gov |
| COBALT | NIH | ncbi.nlm.nih.gov/tools/cobalt/re_cobalt.cgi |
| NCBI Multiple | NIH | ncbi.nlm.nih.gov/projects/msaviewer/ |
| Sequence | | |
| Alignment Viewer | | |
| IP2 | Bruker | integratedproteomics.com |
| Fiji | NIH | fiji.sc/ |
| Exo View Analyzer | Nanoview | accela.eu/nanoview-biosciences |
| 3.1.4 | Bioscience | |
| pClamp 10.7 | Axon | support.moleculardevices.com/s/ |
| | Instruments | |
| miniAnalysis | Bluecell | bluecell.co.kr/theme/theme05/product/product_02_01.php |
| FastQC | QUBES | qubeshub.org/resources/fastqc |
| Trimmomatic | THE USADEL | usadellab.org/cms/?page=trimmomatic |
| | LAB | |
| TopHat | Transcriptomics | ccb.jhu.edu/software/tophat/index.shtml |
| | technologies | |
| HTseq | Dr. Simon | htseq.readthedocs.io/en/latest/ |
| | Anders lab | |
| edgeR | Bioconductor | bioconductor.org/packages/release/bioc/html/edgeR.html |
| Imaris 10.0.0 | Oxford Instruments | imaris.oxinst.com/ |

### Experimental Model and Study Participant Details

All procedures were approved by Northwestern University's Animal Care and Use Committee (IS00009900, IS00010858 and IS00001789) in compliance with US National Institutes of Health standards. The Alix^{+/-} mice were generated by d'Azzo lab³³. The Alix^{fl/fl} mice were generated by Sadoul lab³⁹. The Camk2a-Cre mice were purchased from The Jackson Laboratory (B6.Cg-Tg(Camk2a-cre)T29-1Stl/J, Strain #:005359). Timed pregnant female Wistar rats were purchased from Charles River. We first crossed Alix^{fl/fl} mice with homozygous Camk2a-Cre mice to generate Camk2a-Cre:: Alix^{fl/WT} mice, which were further used as breeders to generate Camk2a-Cre:: Alix^{fl/fl} mice. In all of the experiments, both male and female mice were used.

### Method Details

### Primary Culture of Hippocampal Neurons

Brains of postnatal day 0 Wistar rat or mouse pups were removed rapidly and placed in ice-cold Hank's Balanced Salt Solution (Thermo Fisher Scientific, Cat #14170112). Hippocampi were dissected and incubated with papain for 20 min at 37° C., followed by trituration with fire-polished glass pipettes, and plated in poly-D-lysine (Fisher Scientific, Cat #CB-40210)/laminin (Fisher Scientific, Cat #23-017-015)-coated 60-mm culture dishes (2×10⁶ cells per dish), in 6-well plate (cover glasses, Carolina, Cat #633037, 1.5×10⁶ cells per well), in 12-well plate (5×10⁵ cells per well), 24-well plates (cover glasses, Carolina, Cat #633029, 1.5×10⁵ cells per well). Notably, the Alix^{+/-} mice were used as breeders. Therefore, mouse hippocampal neurons from each P0 pup were cultured individually. The tail of the corresponding mouse pup was used for the genotyping (Transnetyx). Neurons were cultured with Neurobasal medium supplemented with SM1 (STEMCELL Technologies, Cat #05711) and maintained at 37° C. in a humidified 5% CO₂ atmosphere incubator. Cultures were fed three times a week with changing 50% of the medium. Glial cell division was suppressed by addition of 5-fluoro-2-deoxyuridine (20 g/ml; Sigma-Aldrich, Cat #F0503) and uridine (20 μg/ml; Sigma-Aldrich, Cat #U6381) at day four after plating (DIV 4). For EV enrichment, DIV 28 neurons were used. For immunostaining and WB, DIV 21-28 neurons were used. For electrophysiological recording and live cell imaging, DIV 15-18 neurons were used.

### Plasmids and Lentivirus (LV)

The mCherry-Alix plasmid and Notch1-myc were purchased from Addgene (Cat #21504, and Cat #41728). The pz196 plasmid was acquired from Dr. Evangelos Kiskinis lab. The GFP and mCheery plasmids were acquired from Dr. Peter Penzes lab. The mCherry-Alix-S717A and mCherry-Alix-S717D constructs were generated by QuikChange method with following oligoes⁶².

AlixS717A_For:
(SEQ ID NO: 25)
acttgcaacaaGCAattgccagagaacctagtgct
AlixS717A_Rev:
(SEQ ID NO: 26)
gttctctggcaatTGCttgttgcaagtcctttaag
AlixS717D_For:
(SEQ ID NO: 27)
acttgcaacaaGATattgccagagaacctagtgct
AlixS717D_Rev:
(SEQ ID NO: 28)
gttctctggcaatATCttgttgcaagtcctttaag

The mCherry, mCherry-Alix, mCherry-Alix-S717A and mCherry-Alix-S717D LVs were packaged by System Biosciences. The Notch1-myc lentiviruses (LV) were packaged in the Savas lab. LVs were added into primary cultured Alix¹ mouse or rat hippocampal neurons at DIV 4.

### Electrophysiological Recording

14-16 days after plating, coverslips were transferred to a recording chamber positioned under an upright microscope equipped with a 40× objective. They were continuously super fused (1 ml/min) with oxygenated sodium ACSF containing 125 mM NaCl, 2.4 mM KCl, 1.2 mM NaH₂PO₄, 25 mM NaHCO₃, 25 mM glucose, 2 mM CaCl₂), and 1 mM MgCl₂ (pH 7.4; osmolarity between 325 and 335 mosmol-1) and maintained at near physiological temperature (32° C.) on a heated stage. Whole-cell patch clamp recordings were performed using a Multiclamp700B amplifier (Axon Instruments Inc.). Borosilicate glass recording electrodes with resistances of 4-6 MΩ were filled with the following internal solution: 95 mM CsF, 25 mM CsCl, 10 mM Cs-HEPES, 10 mM Cs-EGTA, 2 mM NaCl, 2 mM Mg-ATP, 10 mM QX-314, 5 mM TEA-Cl, 5 mM 4-AP, (pH adjusted to 7.3 with CsOH). Series resistance was continuously monitored using hyperpolarizing voltage steps generated by pClamp 10.7 software (Axon Instruments), and recordings were discarded if there was a >15% change during the course of the experiment. No electronic compensation for series resistance was applied. Recordings were filtered at 2 kHz. All mEPSC recordings were made in the presence of TTX (0.5 μM) and GABAA receptor antagonists, picrotoxin (50 μM) and bicuculline (10 μM). For cLTP induction, perfusion was switched for 5 min to a Magnesium-free ACSF containing Glycine (200 μM). mEPSC were analyzed using miniAnalysis (Bluecell) with a threshold set at 5 pA.

### Neuronal EV Release Induction

Synaptic NMDARs were activated as previously described^{16,63}. Neurons were washed three times with warm Mg²⁺-free glycine solution (pH 7.4, 150 mM NaCl, 5 mM KCl, 200 mM glycine, 2 mM CaCl₂), 10 mM glucose, 10 mM HEPES, 1 μM Strychnine, 20 μM Bicuculline. The osmolarity was adjusted to 300-330 mOsm/l). The neurons were kept in Mg²⁺-free glycine solution for 30 mins at 37° C. in a humidified 5% CO₂ atmosphere incubator. The EV-containing supernatant was then carefully collected to enrich for EVs. As a negative control experiment, we did a treatment of neurons with the Veh solution (pH 7.4, 150 mM NaCl, 5 mM KCl, 1 mM MgCl₂, 2 mM CaCl₂), 10 mM glucose, 10 mM HEPES, 1 μM Strychnine, 20 μM Bicuculline. The osmolarity was adjusted to 300-330 mOsm/l). The Veh solution with which the neurons had been treated was also collected for further analysis.

### SEC-Based EV Enrichment

The EV-containing supernatant was centrifuged at 3000×g for 30 mins at 4° C. to remove residual neurons and large debris. Then, the supernatant was concentrated using a molecular weight cutoff (MWCO) filter (10 KDa, EMD Millipore, Cat #UFC801024) to ˜0.5 ml. The concentrated solution was then fractionated into 10 fractions (0.5 ml in EV-free PBS, MEDIATECH INC CA, Cat #21-040-CV) using a qE-Voriginal-35 nm SEC column (Izon Science, Cat #SP5-USD) with an automated fraction collector V1 (Izon Science, Cat #AFC-V1).

### Quantification of EV Size

Purified EV samples were submitted to Center for Advanced Microscopy/Nikon Imaging Center (Northwestern University) for negative staining EM analysis. Images were acquired with a FEI Tecnai Spirit G2 120 kV transmission EM. EM images were imported into Fiji (NIH, https://imagej.nih.gov/ij/). All visible EV-like particles were measured and no particles were omitted. Before measurement, the images were processed with sharpen function for edge-detection.

### Quantification of EV

For each experimental group, EVs released from three 60 mm dishes of mouse hippocampal neurons were pooled and enriched by the SEC method as described above. Then take 10 μl of the enriched EVs from each sample and dilute them with 990 μl EV-free PBS. The samples were centrifuged at 2500×g for 15 mins. Mix 0.5 ml of each sample with 0.5 ml incubation solution. Load 70 μl of each mixture to an ExoView Mouse Tetraspanin chip (Nanoview Bioscience, Cat #EV-TETRA-M2) and incubate overnight at RT in dark. The next day, unbound EVs were washed off the chip by five 3-min washes with the solution A. After incubating with the block solution for 1 hr (RT), the chips were incubated with anti-tetraspanin mixture (Included in the kit, anti-CD81, CF® 555, anti-CD9, CF® 488A and anti-CD63, CF® 647) for 1 hr on an orbital shaker. The chips were washed with the solution A twice (3 mins each) and the solution B 5 times (3 min each). The chips were then rinsed in MilliQ water to remove excess solution B. Air dry the chips. A NanoView R100 equipment (Nanoview Bioscience) was used to scan the chips. The data was analyzed by ExoView Analyzer 3.1.4 (Nanoview Bioscience).

### EV Labeling

EVs were labeled with the protein dye CFSE or the lipid dye CM-Dil as described previously⁶⁴. EV-containing solution was collected from twelve 60 mm culture dishes (DIV 28, 2×10⁶ cells per dish) as described above. The solution was then concentrated to ˜0.5 ml using a 10 KDa MWCO filter. CFSE (Thermo Fisher Scientific, Cat #C34554) and CM-Dil (Thermo Fisher Scientific, Cat #C7000) dyes were resuspended with DMSO (Sigma-Aldrich, Cat #D2650) and adjusted to 5 mM and 7.5 μM respectively. 8 μl CFSE or 10 μl CM-Dil was added to each 0.5 mL of concentrated EV-containing solution. The mixture was incubated for 1 hour in the dark in a 37° C. water bath. Then, as described above, we enriched labeled EVs and removed excess dyes by SEC fractionation (as above). Labelled EVs exist in SEC F2, which was concentrated to ˜20 μl using a MWCO filter (30 KDa, Millipore Sigma, Cat #MRCF0R030).

### Proteinase k (PK) Treatment of EV

EV-containing solution was collected from twelve 60 mm culture dishes (DIV 28, 2×10⁶ cells per dish) as described above. The solution was then concentrated to ˜0.5 ml using a 10 KDa MWCO filter and divided into two equal parts (˜0.25 ml each). PK (Fisher Scientific, Cat #NC0547027) was added into one part and made final concentration to 100 g/ml. Both parts were incubated for 1 hour in the dark in a 37° C. water bath. Then deactivate PK by heating at 95° C. for 10 mins. Volume each part to 0.5 ml with PBS. Then, label the EVs with CFSE dye, and enrich the labelled EVs by SEC fractionation as shown above. The volume of each part was reduced to ˜20 μl using a 30 KDa MWCO filter.

### Delta^{MAX} Treatment of EV

EVs were collected from twelve 60 mm culture dishes (DIV 28, 2×10⁶ cells per dish) as described above. The solution was then concentrated to ˜0.5 ml using a 10 KDa MWCO filter and divided into two equal parts (˜0.25 ml each). Delta^{MAX} was added into one part and made final concentration to 5 μM. Both parts were incubated for 30 mins in the dark with rotation at 4° C. Then the volume was brought up to 0.5 ml total with PBS. Then, the EVs were enriched by SEC fractionation as described above. The volume of each tube was reduced to ˜20 μl using a 30 KDa MWCO filter.

### Neuronal EV Uptake Assay

The labelled EVs were diluted with 300 μl neuronal culture medium and divided into three equal portions. 100 μl of diluted labelled EVs were added into one well of a 24-well plate of primary cultured hippocampal neurons (DIV 21-28, on cover glasses, 1.5×10⁶ cells per well). The plate was incubated at 37° C. in a humidified 5% CO₂ atmosphere incubator for 30 mins. Then wash three times in D-PBS (Fisher Scientific, Cat #AAJ67802K2) to remove non-absorbed EVs. After being fixed with 4% PFA (in D-PBS, 4% sucrose) for 15 minutes at RT with gentle agitation, immunocytochemistry was performed on the samples.

### Lactate Dehydrogenase (LDH) Assay

After collection of the culture medium, the neurons were treated with either Vehicle (Veh) or Mg²⁺-free glycine solution for 30 mins at 37° C. in a humidified 5% CO₂ atmosphere incubator. A LDH assay kit (Thermo Fisher Scientific, Cat #88953) was then used to measure LDH levels in the culture medium, Veh, or Mg²⁺-free glycine solution with which the neurons were treated. An aliquot of each sample (100 l) was transferred from the culture wells to the wells of a 96-well plate and mixed with 100 μl of the reaction solution provided by the kit. Optical density was measured at 492 nm 45 min later using a Synergy HTX multi-mode microplate reader (Biotek). Background absorbance at 620 nm was subtracted. The maximal releasable LDH in each well was then obtained by a 15 min incubation with 1% Triton X-100 at the end of each experiment.

### Live Cell Imaging

DIV 18 rat hippocampal neurons in 6-well plate (cover glasses, Carolina, Cat #633037, 1.5×10⁶ cells per well) were used for this assay. At DIV 16, neurons were co-transfected with either 1 μg mCherry+GFP or 1 μg mCherry-Alix+GFP plasmids using HilyMax (Dojindo Cat #H357-15). Two days later, the neurons were used for live cell imaging analysis. Neurons (put in a magnetic imaging chamber, Warner Instruments, Cat #QR-40L) were rinsed three times with warm (37° C.) extracellular solution (pH 7.4, 150 mM NaCl, 5 mM KCl, 1 mM MgCl₂, 2 mM CaCl₂), 10 mM glucose, 10 mM HEPES). Then the neurons were placed into a heating stage (37° C.) supplied with a humidified 5% CO₂ atmosphere and were incubated with this solution for 20 mins in a humidified 5% CO₂ atmosphere incubator at 37° C. After washing twice with warm Mg²⁺-free glycine solution, the neurons were incubated with Mg²⁺-free glycine solution for five mins. Then the extracellular solution was put back. Images were captured using a Nikon A1R+ confocal laser microscope and a 100× oil objective lens (also was kept at 37° C. during recording). To minimize bleaching, we only sampled the signals at indicated time points in FIG. 8. The data was imported to Fiji (NIH) for further analysis.

### Immunocytochemistry (ICC)

Neurons (cultured on cover glasses) were washed three times with D-PBS (Fisher Scientific, Cat #AAJ67802K2). Neurons were then fixed with 4% PFA (in D-PBS, 4% sucrose) for 15 minutes at RT with gentle agitation. PFA was quenched by three 5-min washes in PBS (1 M glycine). Three subsequent 5-minute washes with PBS removed glycine. After blocking (10% horse serum, 0.1% Triton X-100 in PBS) for 1 hour, neurons were incubated with primary antibodies overnight at 4° C. with gentle agitation. Following primary antibodies were used: chicken anti-MAP2 (1:5000, Millipore Sigma, Cat #AB5543, RRID: AB_571049), mouse anti-Jag1 (1:1000, Santa Cruz Biotechnology, Cat #sc-390177, RRID: AB_2892141), goat anti-Jag2 (1:1000, Thermo Fisher Scientific, Cat #PA5-47188, RRID: AB_2576459), rabbit anti-Jag2 (1:1000, Cell Signaling Technology, Cat #2210, RRID: AB_823553), goat anti-Dll1 (1:1000, Abcam, Cat #ab85346, RRID: AB_1860332), rabbit anti-Dll1 (1:1000, Cell Signaling Technology, Cat #2588, RRID: AB_2292961), goat anti-Dll4 (1:1000, Thermo Fisher Scientific, Cat #PA5-46974, RRID: AB_2577158), rabbit anti-Dll4 (1:1000, Cell Signaling Technology, Cat #96406, RRID: AB_2800263), guinea pig anti-VGluT1 (1:1000, Millipore Sigma, Cat #AB5905, RRID: AB_2301751), rabbit anti-Homer1 (1:1000, Synaptic Systems, Cat #160 003, RRID: AB_887730), mouse anti-Tsg101 (1:1000, Santa Cruz Biotechnology, Cat #sc-7964, RRID: AB_671392), rabbit anti-Notch1_{ICD} (1:1000, Abcam, Cat #ab52627, RRID: AB_881725), mouse anti-LBPA (1:500, Millipore Sigma, Cat #MABT837), rabbit anti-Notch2_{ICD} (1:1000, Cell Signaling Technology, Cat #5732S, RRID: AB_10693319), mouse anti-PSD95 (1:500, Thermo Fisher Scientific, Cat #MA1-046, RRID: AB_2092361), chicken anti-GFP (1:5000, Abeam, Cat #ab13970, RRID: AB_300798) and mouse anti-myc (1:1000, Santa Cruz Biotechnology, Cat #sc-40, RRID: AB_627268). After six 5-min-washes in PBS, neurons were incubated with corresponding Alexa Fluor secondary antibodies (1:1000, Thermo Scientific, Cat #A11057, A11034, A10037, A-11011, A11029, A11055, A-21437 and Abcam Cat #ab150187) overnight at 4° C. with mild agitation. Then, after six 5-min-washes in PBS, neurons were incubated in 4′,6-diamidino-2-phenylindole (1:1000, DAPI, Sigma-Aldrich, Cat #D9542) solution for 5 mins. Neurons were washed one more time with PBS and mounted with Fluoromount-G (Southern Biotech, Ca #0100-01) on microscope slides.

Images were captured using a Nikon A1R+ confocal laser microscope and 20× or 100× objective lens. For each coverslip, area was randomly selected. Images were processed and analyzed by Fiji (NIH) with plugins Ratioplus, Colocalization Finder, and JACOP. Imaris (Oxford Instruments) 10.0.0 was used for spine analysis.

### Using EVs to Treat Neurons

PK-related experiments: enriched EVs from twelve 60 mm culture dishes (DIV 28, 2×10⁶ cells per dish) were divided into two equal parts. One part of EVs were treated with PK as mentioned above. SEC fractionation was used to remove excess PK. Veh (20 μl PBS), EVs (20μ) and PK-treated EVs (20 μl) were each added to three wells of cultured neurons in an 8-well plate, respectively.

Delta^{MAX}-related experiments: enriched EVs from twelve 60 mm culture dishes (DIV 28, 2×10⁶ cells per dish) were divided into two equal parts. One half of the EVs were treated with Delta^{MAX} as mentioned above. SEC fractionation was used to remove excess Delta^{MAX}. Veh (20 μl PBS), EVs (20), Delta^{MAX}-treated EVs (20 μl) and Delta^{MAX} (5 μM in 20 μl PBS) were each added to four wells of cultured neurons in an 8-well plate, respectively.

The plates were incubated at 37° C. in a humidified 5% CO₂ atmosphere incubator for 1 hr. The neurons were harvested for WB analysis.

### Pharmacological Treatments of Neurons

Neurons were pretreated with DMSO (1:1000) or dynasore (80 μM, Sigma-Aldrich, Cat #SML0340) or H89 (20 μM, Sigma-Aldrich, Cat #B1427) or staurosporine (1 μM, Tocris, Cat #1285) or APV (50 μM, Sigma-Aldrich, Cat #A5282) or KN93 (10 μM, Sigma-Aldrich, Cat #K1385) or AIP (1 μM, Anaspec, Cat #AS-64929) for 30 mins at 37° C. in a humidified 5% CO₂ atmosphere incubator. Then, neuron culture medium was carefully removed from dishes. The collected medium was then kept at 37° C. in a humidified 5% CO₂ atmosphere incubator. The neurons were washed three times with Veh or Mg²⁺-free glycine and incubated for 5 min with solutions containing different inhibitors. Then neurons were washed twice with Veh solution, the saved culture medium was returned and maintained in incubator for assigned durations (37° C., 5% CO₂).

### Poly-A RNA Sequencing

Total RNA was extracted from neurons using RNeasy mini Kit (Qiagen, Cat #74104). Ribosomal RNA was depleted with RiboZero kit (Epicenter). Poly(A)-enriched RNA were separated by three rounds of Oligo(dT) magnetic beads (Thermo). For each time point, RNA from two 60-mm dishes of neurons were pooled together. Sequence quality was assessed using FastQC, and quality trimming was done using Trimmomatic. RNA-seq reads were aligned to the rat genome (iGenomes UCSC version m6) using TopHat v2.0.9, and only uniquely mapped reads with at most two mismatches were considered for downstream analysis. Gene count tables were constructed using HTseq and used as input for edgeR 3.0.8.

### Subcellular Fractionation

Adult mouse cortexes were rinsed and dissected in solution A (5 mM HEPES pH 7.4, 1 mM MgCl₂, 0.5 mM CaCl₂), 1 mM DTT, 0.32 M sucrose and protease and phosphatase inhibitor set (Thermo Fisher Scientific, Cat #: 78443_3670527377)) on ice. Then the tissues were homogenized with an electronic homogenizer (Glas-Col, Cat #099C-K54). Spin down the homogenates at 1,400×g for 10 mins (4° C.). Set aside the supernatant. Then we resuspended the pellets in 20 ml solution A. The diluted homogenates were centrifuged at 710×g for 10 mins (4° C.). The pellet is P1. We combined and mixed the supernatant and the saved supernatant as S1. S1 were centrifuged at 13,800×g for 10 mins (4° C.). The supernatant is S2. Then we resuspended the pellets (P2) in solution B (6 mM Tris pH 8.1, 0.32 M sucrose, 1 mM EDTA, 1 mM EGTA, 1 mM DTT with protease and phosphatase inhibitors). Then we layered the homogenized pellets on discontinuous sucrose gradient (1.2 M. 1.0 M and 0.85 M sucrose in 6 mM Tris pH 8.1) and centrifuged it at 82,000×g for two hours (4° C.). Collect the layer between the 1.0 M and 1.2 M interface and dilute it in 2.5× volume with 6 mM Tris pH 8.1. Then centrifuge it at 200,000×g for 30 mins (4° C.). The pellet is synaptosome (SS). Then SS were incubated in ice cold solution C (50 mM HEPES pH 8.1, 2 mM EDTA, 0.5% Triton X) for 15 mins rotation in 4° C. and then centrifuged at 32,000×g for 20 mins (4° C.). The supernatant is presynaptic fraction (Pre). The pellet is resuspended in in ice cold solution C for 15 mins one more time and then centrifuged at 32,000×g for 20 mins (4° C.). The pellet is postsynaptic density (PSD) fraction. BCA (Thermo Fisher Scientific, Cat #23225) all the fraction.

### Western Blotting (WB)

Each SEC fraction was further concentrated to ˜40 μl using a MWCO filter (30 KDa, Millipore Sigma, Cat #MRCF0R030). An equal volume of 2×SDS loading buffer was then added to the sample and mixed. The mixture was heated at 95° C. for 5 mins. Each SEC fraction sample was divided into two equal parts for WB and silver staining analyses.

Neuron samples were collected and resuspended in a RIPA lysis buffer (50 mM Tris, 150 mM NaCl, 0.1% SDS, 1 mM EDTA, 0.5% sodium deoxycholate, 1% Triton X-100, 1× protease inhibitor cocktail (Thermo Fisher Scientific, Cat #78443), 1× phosphatase inhibitor (Thermo Fisher Scientific, Cat #78420), pH 7.4). The resuspended lysates were vortexed for 20 s and then incubated on ice for 30 min and centrifuged at 16,200 g for 15 mins. The supernatants were mixed with 4×SDS loading buffer at a 3:1 ratio. The mixtures were boiled at 95° C. for 5 mins.

Dissected mouse hippocampi were homogenized in RIPA lysis buffer (50 mM Tris, 150 mM NaCl, 0.1% SDS, 1 mM EDTA, 0.5% sodium deoxycholate, 1% Triton X-100, 1× protease inhibitor cocktail (Thermo Fisher Scientific, Cat #78443), 1× phosphatase inhibitor (Thermo Fisher Scientific, Cat #78420), pH 7.4) with an electronic homogenizer (Glas-Col, Cat #099C-K54). The resuspended lysates were vortexed for 20 s and then incubated on ice for 30 min and centrifuged at 16,200 g for 15 mins. Samples were further cleaned up by methanol chloroform precipitation. The protein pellets were resuspended in 1×SDS loading buffer and boiled at 95° C. for 5 mins.

WedgeWell™ Tris-Glycine Mini Protein Gels (Fisher Scientific, Cat #XP04125BOX, XP04120BOX and XV00100PK20) were used for electrophoresis. After electrophoresis, proteins were transferred onto nitrocellulose membranes (Thermo Fisher, Cat #4500002). For WB, the membranes were blocked with 10% bovine serum albumin (BSA, Jackson immunoResearch laboratories, Cat #001-000-162) in TBST solution (Tris-buffered saline, 0.1% Tween 20) for one hour in room temperature (RT). Then membranes were incubated with primary antibodies in incubation solution (3% BSA in TBST) overnight at 4° C. with mild agitation. Following primary antibodies were used: mouse anti-Alix (1:1000, Cell Signaling Technology, Cat #2171S, RRID: AB_2299455), rabbit anti-Alix (1:1000, Cell Signaling Technology, Cat #18269, RRID: AB_2798796), mouse anti-Tsg101 (1:1000, Santa Cruz Biotechnology, Cat #sc-7964, RRID: AB_671392), mouse anti-CD81 (1:1000, Santa Cruz Biotechnology, Cat #sc-166029, RRID: AB_2275892), rabbit anti-Notch1_{ICD} (1:1000, Abcam, Cat #ab52627, RRID: AB_881725), rabbit anti-Notch2_{ICD} (1:1000, Cell Signaling Technology, Cat #5732S, RRID: AB_10693319), rabbit anti-activated Notch1 (1:1000, Abcam, Cat #ab52301, RRID: AB_881726), rabbit anti-activated Notch2 (1:1000, MyBioSource, Cat #MBS9410510), rabbit anti-Sdcbp (1:1000, Abcam, Cat #ab19903, RRID: AB_445200 and Cell Signaling Technology, Cat #27964), rabbit anti-GM130 (1:1000, Abcam, Cat #ab52649, RRID: AB_880266), rabbit anti-Hes1 (1:1000, Cell Signaling Technology, Cat #11988S, RRID: AB_2728766 and Abcam, Cat #ab71559, RRID: AB_1209570), chicken anti-Tuj1 (1:2000, Novus Biologicals, Cat #NB100-1612, RRID: AB_10000548), rabbit anti-p-S/T (1:1000, Cell Signaling Technology, Cat #9624S, RRID: AB_331817), mouse anti-PSD95 (1:2000, Thermo Fisher Scientific, Cat #MA1-046, RRID: AB_2092361), rabbit anti-PSD95(pS295) (1:1000, Abcam, Cat #ab76108, RRID: AB_1310621), rabbit anti-GAPDH (1:2000, Cell Signaling Technology, Cat #2118, RRID: AB_561053), rabbit anti-GluA1 (1:1000, EMD Millipore, Cat #PC246, RRID: AB_564636) and guinea pig anti-Bassoon (1:1000, Synaptic Systems, Cat #141 318, RRID: AB_2927388). After 3×10 mins intense wash in TBST solution, membranes were incubated with corresponding secondary antibodies for one hour at RT with mild agitation. Following secondary antibodies were used: goat-anti-rabbit poly-HRP (1:2000, Thermo Scientific, Cat #32260), goat-anti-mouse poly-HRP (1:2000, Thermo Scientific, Cat #32230), goat-anti-chicken HRP (1:2000, Abcam, Cat #ab97135) and Goat anti-Guinea Pig poly-HRP (1:2000, Fisher Scientific, Cat #OB6090-05). Then membranes were washed 3×10 mins in TBST and developed with SuperSignal West Pico Chemiluminescent Substrate (Thermo Fisher, Cat #34578) and imaged on a Chemidoc XRS system (Bio-Rad). Fiji (NIH) was used for quantification.

### Immunoprecipitation (IP)

Three-five 60 mm-dishes of primary cultured rat hippocampal neurons were collected and re-suspended in IP buffer (20 mM Tris-Cl, pH 7.4, 150 mM NaCl, 1% Triton X-100, 1 mM EDTA, 3 mM NaF, 1 mM β-glycerophosphate, 1 mM sodium orthovanadate, 2 mM N-ethylmaleimide and 10% glycerol) with complete protease and phosphatase inhibitor set (Thermo Fisher, Cat #78443). After incubating on ice for 30 mins, the lysates were centrifuged at 13,000×g for 15 mins (4° C.). Supernatant were collected and precleared with 100 μl Protein G Sepharose® 4 Fast Flow (Thermo Fisher, Cat #45-000-116) for one hour at 4° C. Then samples were incubated with two g mouse anti-Alix (Cell Signaling Technology, Cat #2171) or IgG (Sigma-Aldrich, Cat #15381) overnight at 4° C. The next day, 50 μl Protein G Sepharose® 4 Fast Flow were added to the samples and incubated for 2 hours at 4° C. Then, the beads were washed three times with IP buffer. The immunoprecipitants were eluted with 2×SDS loading buffer.

### Lambda Phosphatase Assay

Alix immunoprecipitates from neurons treated with Veh or Mg²⁺-free glycine solution were resuspended in lambda phosphatase assay buffer (50 mM Tris-HCl pH 7.8, 5 mM dithiothreitol, 2 mM MnCl₂, 100 g/ml BSA) with or without 1600 U of lambda phosphatase (New England Biolabs, Cat #P0753S). After 60 minutes at 30° C., the reactions were stopped by adding 2×SDS loading buffer and processed for WB with the indicated antibodies.

### Silver Staining

We used Pierce Silver Stain Kit (Thermo Fisher, Cat #24612) for silver staining. Gels were washed 2×5 mins in ultrapure water and then fixed with 2×15 mins in 30% ethanol: 10% acetic acid solution. Then the fixed gels were washed 2×5 mins with 10% ethanol and 2×5 mins in ultrapure water. Then the gels were sensitized for 1 min and washed twice 2×1 min with water. Then, the gels were stained for 30 mins and washed 2×20 secs with ultrapure water. The gels were developed for 1-3 mins until bands appear and stopped with 5% acetic acid for 10 mins. A Chemidoc XRS system (Bio-Rad) was used for imaging.

### Immunohistochemistry (IHC) Staining

Mice were anesthetized and transcardially perfused with ice old PBS followed by 4% PFA (in PBS, pH 7.4). Dissected brains were cryoprotected in 30% sucrose solution and then buried in embedding medium (Sakura, Cat #4583). Sagittal sections (35 μm) were obtained using a cryostat microtome. Slices were washed 3×5 mins in glycine solution (1M glycine in PBS, pH 7.4) to block unreacted PFA. Slices were further washed 3×5 mins in PBS to remove glycine. For permeabilization, slices were incubated in Triton X-100 (0.5% in PBS, pH7.4) solution for 1 hr at RT. After blocking (10% horse serum, 0.5% Triton X-100 in PBS, pH7.4) for 1 hr, slides were incubated with primary antibodies at 4° C. for 48 hrs with mild agitation. Following primary antibodies were used: rabbit anti-Notch1_{ICD} (1:500, Abcam, Cat #ab52627), rabbit anti-Notch2_{ICD} (1:500, Cell Signaling Technology, Cat #5732S) and chicken anti-Tuj1 (1:500, Novus Biologicals, Cat #NB100-1612). After 9×5 min washing in PBS, slides were incubated with corresponding Alexa Fluor secondary antibodies (1:1000, Thermo Scientific, Cat #A11034 and Abcam, Cat #ab150187) overnight at 4° C. with mild agitation. Then, after 8×5 mins washing in PBS, slides were incubated in 4′,6-diamidino-2-phenylindole (1:1000, DAPI, Sigma-Aldrich, Cat #D9542) solution for 10 mins. The slides were washed 5 mins with PBS and mounted with ProLong™ Diamond Antifade Mountant (Thermo Fisher Scientific, Cat #P36970) on microscope slides.

Images were captured using a Nikon A1R+ confocal laser microscope and 100× objective lens. For each mouse, brain slices were randomly selected. Images were processed and analyzed by Fiji (NIH).

### Sample Preparation or MS Analysis

Trichloroacetic acid (TCA, Sigma-Aldrich, Cat #T0699) precipitation was used to clean and precipitate proteins from SEC fractionation samples. Proteins from neuron samples and mouse hippocampal samples were extracted and precipitated as described in Western blotting (WB)_section. Protein pellets were resuspended in 8 M urea (Thermo Fisher Scientific, Cat #29700) prepared in 100 mM ammonium bicarbonate solution (Fluka, Cat #09830). Dithiothreitol (DTT, DOT Scientific Inc, Cat #DSD11000) was applied to a final concentration of 5 mM. After incubation at RT for 20 mins, iodoacetamide (IAA, Sigma-Aldrich, Cat #11149) was added to a final concentration of 15 mM and incubated for 20 mins at RT in the dark. Excess IAA was quenched with DTT for 15 mins. Samples were diluted with 100 mM ammonium bicarbonate solution, and digested for three hrs with Lys-C protease (1:100, Thermo Fisher Scientific, Cat #90307_3668048707) at 37° C. Trypsin (1:100, Promega, Cat #V5280) was then added for overnight incubation at 37° C. with intensive agitation (1000 rpm). The next day, reaction was quenched by adding 1% trifluoroacetic acid (TFA, Fisher Scientific, Cat #04902-100). The SEC fraction samples were desalted using ZipTip (Thermo Fisher-Pierce, Cat #87784). The neuron and hippocampal samples were desalted using Peptide Desalting Spin Columns (Thermo Fisher Scientific, Cat #89852). All samples were vacuum centrifuged to dry.

### Enrichment of Phosphorylated Peptides

Neurons were stimulated with Mg²⁺-free glycine solution for 10 mins. Peptides were prepared as described above. The High-Select™ TiO₂ Phosphopeptide Enrichment Kit (Thermo Fisher Scientific, Cat #A32993) was used to enrich phosphorylated peptides following the manufacturer's specifications.

### Tandem Mass Tag (TMT) Labeling

A TMT labeling protocol was developed based upon previously described methods⁶⁸. C18 column-desalted peptides were resuspended with 100 mM HEPES pH 8.5 and the concentrations were measured by micro BCA kit (Fisher Scientific, Cat #PI23235). For each sample, 100 μg of peptide labeled with TMT reagent (0.4 mg, dissolved in 40 μl anhydrous acetonitrile, Thermo Fisher Scientific, Cat #A44520) and made at a final concentration of 30% (v/v) acetonitrile (ACN). Following incubation at RT for 2 hrs with agitation, hydroxylamine (to a final concentration of 0.3% (v/v)) was added to quench the reaction for 15 min. TMT-tagged samples were mixed at a 1:1:1:1:1:1:1:1 ratio. Combined sample was vacuum centrifuged to dryness, resuspended, and subjected to HyperSep C18 Cartridges (ThermoFisher Scientific, Cat #60108-302).

### Peptide Fractionation

A high pH reverse-phase peptide fractionation kit (Thermo Fisher Scientific, Cat #84868) was used to obtain eight fractions (5.0%, 10.0%, 12.5%, 15.0%, 17.5%, 20.0%, 22.5%, 25.0% and 50% of ACN in 0.1% triethylamine solution). The high pH peptide fractions were directly loaded into the autosampler for MS analysis without further desalting.

### Tandem Mass Spectrometry

Three micrograms of each fraction or sample were auto-sampler loaded with an UltiMate 3000 HPLC pump onto a vented Acclaim Pepmap 100, 75 um×2 cm, nanoViper trap column coupled to a nanoViper analytical column (Thermo Fisher Scientific, Cat #: 164570, 3 μm, 100 Å, C18, 0.075 mm, 500 mm) with stainless steel emitter tip assembled on the Nanospray Flex Ion Source with a spray voltage of 2000 V. An Orbitrap Fusion (Thermo Fisher Scientific) was used to acquire all the MS spectral data. Buffer A contained 94.785% H₂O with 5% ACN and 0.125% FA, and buffer B contained 99.875% ACN with 0.125% FA. For TMT-MS experiments, the chromatographic run was for 4 hours in total with the following profile: 0-7% for 7, 10% for 6, 25% for 160, 33% for 40, 50% for 7, 95% for 5 and again 95% for 15 mins receptively. For other MS experiments, the chromatographic run was for 2 hours in total with the following profile: 2-8% for 6, 8-24% for 64, 24-36% for 20, 36-55% for 10, 55-95% for 10, 95% for 10 mins.

A multiNotch MS³-based TMT method was used to analyze all the TMT samples⁶⁸⁻⁷⁰. The scan sequence began with an MS¹ spectrum (Orbitrap analysis, resolution 120,000, 400-1400 Th, AGC target 2×10⁵, maximum injection time 200 ms). MS² analysis, ‘Top speed’ (2 s), Collision-induced dissociation (CID, quadrupole ion trap analysis, AGC 4×10³, NCE 35, maximum injection time 150 ms). MS³ analysis, top ten precursors, fragmented by HCD prior to Orbitrap analysis (NCE 55, max AGC 5×10⁴, maximum injection time 250 ms, isolation specificity 0.5 Th, resolution 60,000).

The CID-MS² method was used for other experiments. Briefly, ion transfer tube temp=300° C., Easy-IC internal mass calibration, default charge state=2 and cycle time=3 s. Detector type set to Orbitrap, with 60K resolution, with wide quad isolation, mass range=normal, scan range=300-1500 μm/z, max injection time=50 ms, AGC target=200,000, microscans=1, S-lens RF level=60, without source fragmentation, and datatype=positive and centroid. MIPS was set as on, included charge states=2-6 (reject unassigned). Dynamic exclusion enabled with n=1 for 30 s and 45 s exclusion duration at 10 ppm for high and low. Precursor selection decision=most intense, top 20, isolation window=1.6, scan range=auto normal, first mass=110, collision energy 30%, CID, Detector type=ion trap, OT resolution=30K, IT scan rate=rapid, max injection time=75 ms, AGC target=10,000, Q=0.25, inject ions for all available parallelizable time.

### MS Data Analysis and Quantification

Protein identification/quantification and analysis were performed with Integrated Proteomics Pipeline—IP2 (Bruker, Madison, WI. http://www.integratedproteomics.com) using ProLuCID^{71,72}, DTASelect2^{73,74}, Census and Quantitative Analysis (For TMT-MS experiments). Spectrum raw files were extracted into MS1, MS2 and MS3 (For TMT experiments) files using RawConverter (http://fields.scripps.edu/downloads.php). The tandem mass spectra were searched against UniProt mouse (downloaded on Oct. 26, 2020) or rat (downloaded on Mar. 25, 2014) protein databases⁷⁵ and matched to sequences using the ProLuCID/SEQUEST algorithm (ProLuCID version 3.1) with 5 ppm peptide mass tolerance for precursor ions and 600 ppm for fragment ions. The search space included all fully and half-tryptic peptide candidates within the mass tolerance wdindow with no-miscleavage constraint, assembled, and filtered with DTASelect2 through IP2. To estimate peptide probabilities and false-discovery rates (FDR) accurately, a target/decoy database containing the reversed sequences of all the proteins appended to the target database was used⁷⁵. Each protein identified was required to have a minimum of one peptide of minimal length of six amino acid residues; however, this peptide had to be an excellent match with an FDR<1% (<5% for TiO₂ column-enriched samples) and at least one excellent peptide match. After the peptide/spectrum matches were filtered, it was estimated that the peptide FDRs were ≤1% for each sample analysis. Resulting protein lists include subset proteins to allow for consideration of all possible protein forms implicated by at least two given peptides identified from the complex protein mixtures. Then, Census and Quantitative Analysis in IP2 was used for protein quantification of TMT-MS experiments and protein quantification was determined by summing all TMT report ion counts. For SEC samples, static modification: 57.02146 C for carbamidomethylation; differential modifications: 15.9949 M for oxidation on M. For neuron samples to detect phosphorylated peptides: static modification: 57.02146 C for carbamidomethylation; differential modifications: 15.9949 M for oxidation on M, 79.9663 STY for phosphorylation on S or T or Y residues. For TMT experiments: static modification: 57.02146 C for carbamidomethylation, 304.2071 for 16-plex TMT tagging; differential modifications: 15.9949 M for oxidation on M, 304.2071 for N-terminal 16-plex TMT tagging, 42.0106 for N-terminal Acetylation. More than one proteoform may be encoded by a single gene. To minimize recalculation of the same peptides into multiple proteoforms, the peptides encoded by the same gene were grouped and quantified and analyzed together.

### Determination of EV Proteins

To determine whether each protein was a true EV protein or a contaminant, the distribution of each protein across all SEC fractions was examined. True EV proteins must meet the following three criteria.
(1) Compared to all other fractions, SEC F2 has significantly higher number of spec counts.
(2) At least detected in two biological replicates in SEC F2.
(3) Should not be detected in SEC F7-10. These fractions contain only small size particles such as protein aggregates and lipid droplets, which are much smaller than EVs.

### Quantification and Statistical Analysis

Spyder (MIT, Python 3.7, libraries, ‘pandas’, ‘numpy’, ‘scipy’, ‘statsmodels’ and ‘bioinfokit’) was used for data statistical analyses. RStudio (version, 1.2.1335, packages, ‘tidyverse’, ‘pheatmap’) was used for data virtualization. The Database for Annotation, Visualization and Integrated Discovery (DAVID) (https://david.ncifcrf.gov/) was used for protein functional annotation analysis. COBALT was used to run the alignment 77. NCBI Multiple Sequence Alignment Viewer (Version 1.15.0) was used for alignment virtualization. The statistical details of experiments can be found in the figure legends, including, the name of statistical test, the number of replicates, and exact p-values. All data points are presented in the figures. Statistical significance was established through appropriate statistical tests and is indicated as follows: ‘NS’ for p-value>0.05, * for p-value<0.05, ** for p-value<0.01 and *** for p-value<0.001.

### REFERENCES

16. Lu, W., Man, H., Ju, W., Trimble, W. S., MacDonald, J.F., and Wang, Y.T. (2001). Activation of synaptic NMDA receptors induces membrane insertion of new AMPA receptors and LTP in cultured hippocampal neurons. Neuron 29, 243-254. 10.1016/s0896-6273(01)00194-5.
17. Boing, A.N., van der Pol, E., Grootemaat, A.E., Coumans, F.A., Sturk, A., and Nieuwland, R. (2014). Single-step isolation of extracellular vesicles by size-exclusion chromatography. J Extracell Vesicles 3. 10.3402/jev.v3.23430.
25. Schroeter, E.H., Kisslinger, J.A., and Kopan, R. (1998). Notch-1 signalling requires ligand-induced proteolytic release of intracellular domain. Nature 393, 382-386. 10.1038/30756.
26. Gonzalez-Perez, D., Das, S., Antfolk, D., Ahsan, H.S., Medina, E., Dundes, C.E., Jokhai, R.T., Egan, E.D., Blacklow, S.C., Loh, K.M., et al. (2023). Affinity-matured DLL4 ligands as broad-spectrum modulators of Notch signaling. Nat Chem Biol 19, 9-17. 10.1038/s41589-022-01113-4.
33. Campos, Y., Qiu, X., Gomero, E., Wakefield, R., Homer, L., Brutkowski, W., Han, Y.G., Solecki, D., Frase, S., Bongiovanni, A., and d'Azzo, A. (2016). Alix-mediated assembly of the actomyosin-tight junction polarity complex preserves epithelial polarity and epithelial barrier.

Nat Commun 7, 11876. 10.1038/ncomms11876.
39. Laporte, M.H., Chatellard, C., Vauchez, V., Hemming, F.J., Deloulme, J.C., Vossier, F., Blot, B., Fraboulet, S., and Sadoul, R. (2017). Alix is required during development for normal growth of the mouse brain. Sci Rep 7, 44767. 10.1038/srep44767.
44. Laporte, M.H., Chi, K.I., Caudal, L.C., Zhao, N., Schwarz, Y., Rolland, M., Martinez-Hernandez, J., Martineau, M., Chatellard, C., Denarier, E., et al. (2022). Alix is required for activity-dependent bulk endocytosis at brain synapses. PLoS Biol 20, e3001659. 10.1371/journal.pbio.3001659.
63. Araki, Y., Zeng, M., Zhang, M., and Huganir, R.L. (2015). Rapid dispersion of SynGAP from synaptic spines triggers AMPA receptor insertion and spine enlargement during LTP.

Neuron 85, 173-189. 10.1016/j.neuron.2014.12.023.
68. Weekes, M.P., Tomasec, P., Huttlin, E.L., Fielding, C.A., Nusinow, D., Stanton, R.J., Wang, E.C., Aicheler, R., Murrell, I., Wilkinson, G.W., et al. (2014). Quantitative temporal viromics: an approach to investigate host-pathogen interaction. Cell 157, 1460-1472. 10.1016/j.cell.2014.04.028.
69. McAlister, G.C., Nusinow, D.P., Jedrychowski, M.P., Wuhr, M., Huttlin, E.L., Erickson, B.K., Rad, R., Haas, W., and Gygi, S.P. (2014). MultiNotch MS3 enables accurate, sensitive, and multiplexed detection of differential expression across cancer cell line proteomes. Anal Chem 86, 7150-7158. 10.1021/ac502040v.
70. Ting, L., Rad, R., Gygi, S.P., and Haas, W. (2011). MS3 eliminates ratio distortion in isobaric multiplexed quantitative proteomics. Nat Methods 8, 937-940. 10.1038/nmeth.1714.
71. Eng, J.K., McCormack, A.L., and Yates, J.R. (1994). An approach to correlate tandem mass spectral data of peptides with amino acid sequences in a protein database. J Am Soc Mass Spectrom 5, 976-989. 10.1016/1044-0305(94)80016-2.
72. Xu, T., Park, S.K., Venable, J.D., Wohlschlegel, J.A., Diedrich, J.K., Cociorva, D., Lu, B., Liao, L., Hewel, J., Han, X., et al. (2015). ProLuCID: An improved SEQUEST-like algorithm with enhanced sensitivity and specificity. J Proteomics 129, 16-24. 10.1016/j.jprot.2015.07.001.
73. Cociorva, D., D, L.T., and Yates, J.R. (2007). Validation of tandem mass spectrometry database search results using DTASelect. Curr Protoc Bioinformatics Chapter 13, Unit 13 14. 10.1002/0471250953.bi1304s16.
74. Tabb, D. L., McDonald, W.H., and Yates, J.R., 3rd (2002). DTASelect and Contrast: tools for assembling and comparing protein identifications from shotgun proteomics. J Proteome Res 1, 21-26.
75. UniProt, C. (2015). UniProt: a hub for protein information. Nucleic Acids Res 43, D204-212. 10.1093/nar/gku989.

### Example 2

This Example demonstrates that glycine-induced extracellular vesicles (Gly-EVs) can be used to deliver mRNA cargo to recipient cells, where mRNA is translated into functional proteins.

### Methods

Neuronal extracellular vesicle release was induced by three different mechanisms: forskolin, DHPG, or glycine.

### Forskolin-Induced Neuronal EV Release Induction

Neurons were washed three times with warm Mg²⁺-free forskolin solution (pH 7.4, 150 mM NaCl, 5 mM KCl, 20 μM forskolin, 2 mM CaCl₂), 10 mM glucose, 10 mM HEPES. The osmolarity was adjusted to 300-330 mOsm/l). The neurons were kept in Mg²⁺-free forskolin solution for 30 mins at 37° C. in a humidified 5% CO₂ atmosphere incubator. The EV-containing supernatant was then carefully collected to enrich for EVs.

### DHPG-Induced Neuronal EV Release Induction

Neurons were washed three times with warm DHPG solution (pH 7.4, 150 mM NaCl, 5 mM KCl, 100 M μDHPG, 2 mM CaCl₂), 2 mM MgCl₂, 10 mM glucose, 10 mM HEPES. The osmolarity was adjusted to 300-330 mOsm/l). The neurons were incubated in DHPG solution for 30 mins at 37° C. in a humidified 5% CO₂ atmosphere incubator. The EV-containing supernatant was then carefully collected and used to enrich the EVs.

### Neuronal EV Release Induction

Neurons were washed three times with warm Mg²⁺-free glycine solution (pH 7.4, 150 mM NaCl, 5 mM KCl, 200 mM glycine, 2 mM CaCl₂), 10 mM glucose, 10 mM HEPES, 1 μM Strychnine, 20 μM Bicuculline. The osmolarity was adjusted to 300-330 mOsm/l). The neurons were kept in Mg²⁺-free glycine solution for 30 mins at 37° C. in a humidified 5% CO₂ atmosphere incubator. The EV-containing supernatant was then carefully collected to enrich for EVs.

### SEC-Based EV Enrichment

The EV-containing supernatant was centrifuged at 3000×g for 30 mins at 4° C. to remove residual neurons and large debris. Then, the supernatant was concentrated using a molecular weight cutoff (MWCO) filter (10 KDa, EMD Millipore, Cat #UFC801024) to ˜0.5 ml. The concentrated solution was then fractionated into 10 fractions (0.5 ml in EV-free PBS, MEDIATECH INC CA, Cat #21-040-CV) using a qE-Voriginal-35 nm SEC column (Izon Science, Cat #SP5-USD) with an automated fraction collector V1 (Izon Science, Cat #AFC-V1). Fraction 2 was collected as EVs. Fraction 10 was used as negative control.

### Using Different Doses of EVs to Treat Neurons

Enriched Gly-EVs from 72 culture dishes (60 mm, DIV 28, 2×10⁶ cells per dish) were divided into four doses in a 1:2:4:8 ratio. Each dose was then split into three equal parts. One part from each EV dose was used to treat a corresponding well of cultured neurons in a 6-well plate (DIV 21, 1.5×10⁶ cells per dish). The plates were incubated at 37° C. in a humidified 5% CO₂ atmosphere incubator for 1 hr. The neurons were harvested for TMT-MS analysis.

### EV Dose-Response Relationship Modeling

The Hill dose-response model was applied to evaluate protein synthesis in recipient neurons induced by Gly-EV mRNAs. Five relative EV doses were tested: 0, 1, 2, 4, and 8. Protein levels were quantified using TMT-MS, and the mean protein levels from three biological replicates were used for model fitting. Outliers were identified using Grubb's test. The model was constructed with the Python packages NumPy and scipy.optimize. R-squared values were used for evaluating the fitting.

### Results

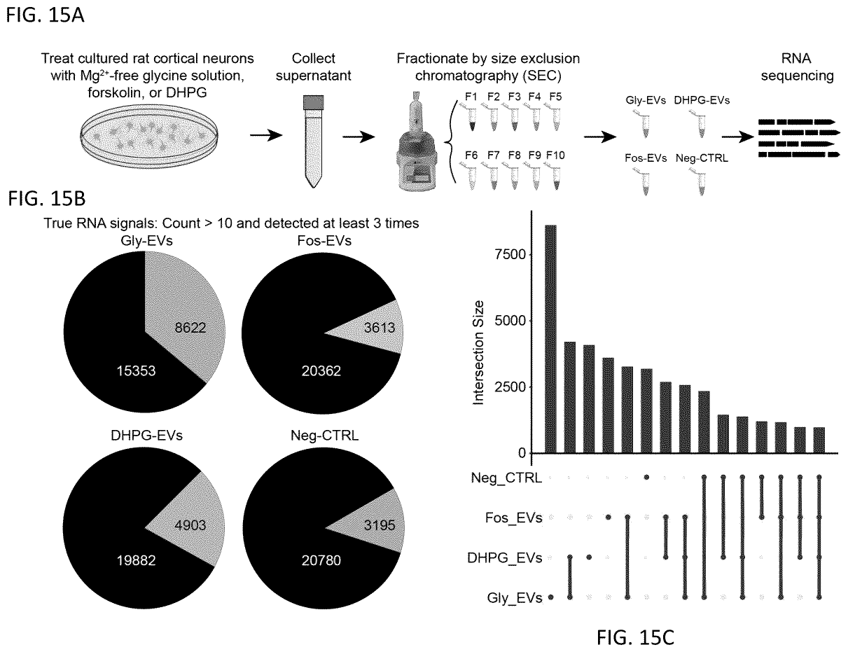

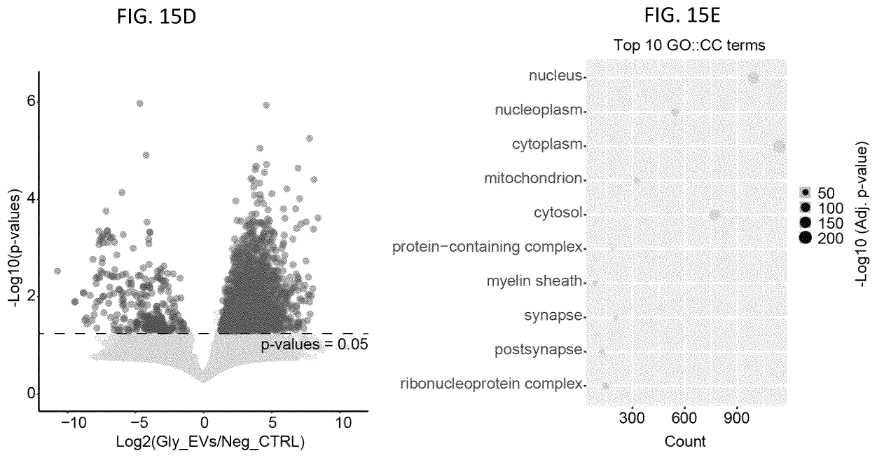

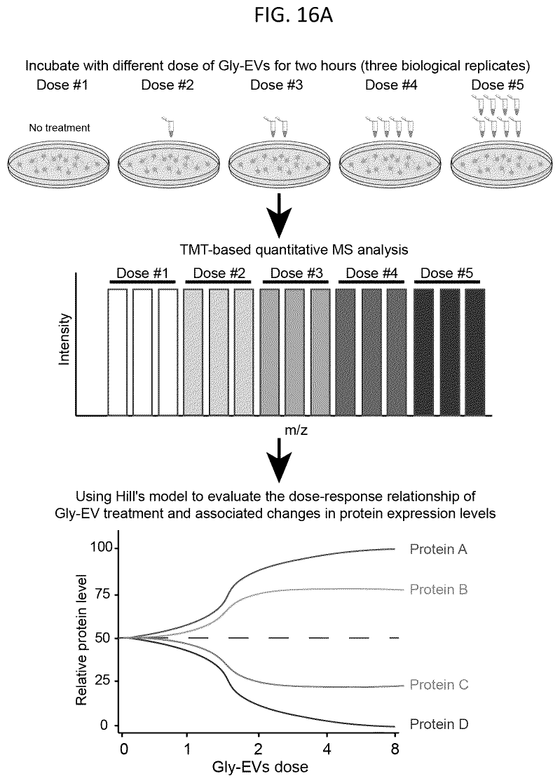

To characterize the mRNA cargo in Gly-EVs, Gly-EVs were isolated and used for bulk RNA sequencing. For comparison, forskolin-induced EVs (Fos-EVs) and DHPG-induced EVs (DHPG-EVs) were also prepared (FIG. 15A). Notably, Gly-EVs exhibited a significantly greater diversity of mRNA species compared to both the other EV types and the negative control (FIG. 15B-D). Gene annotation analysis of the mRNAs enriched in Gly-EVs revealed that a substantial proportion encode synaptic proteins.

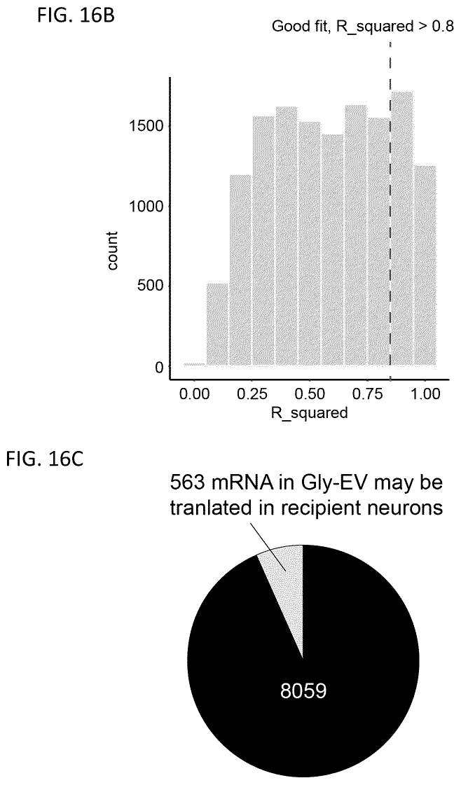

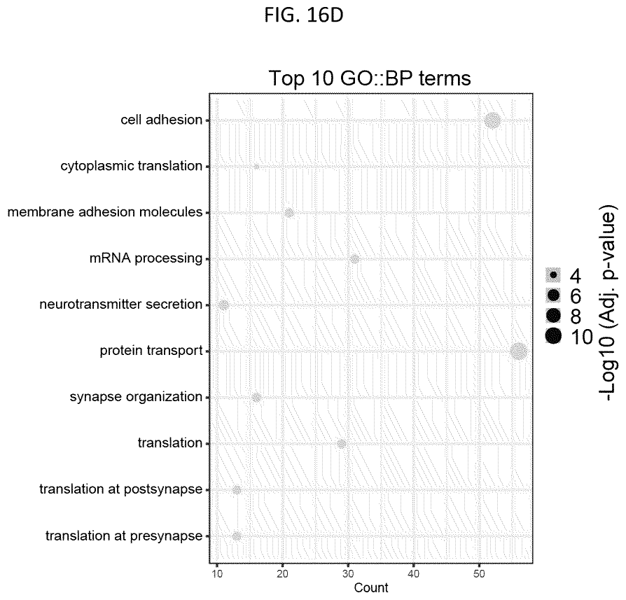

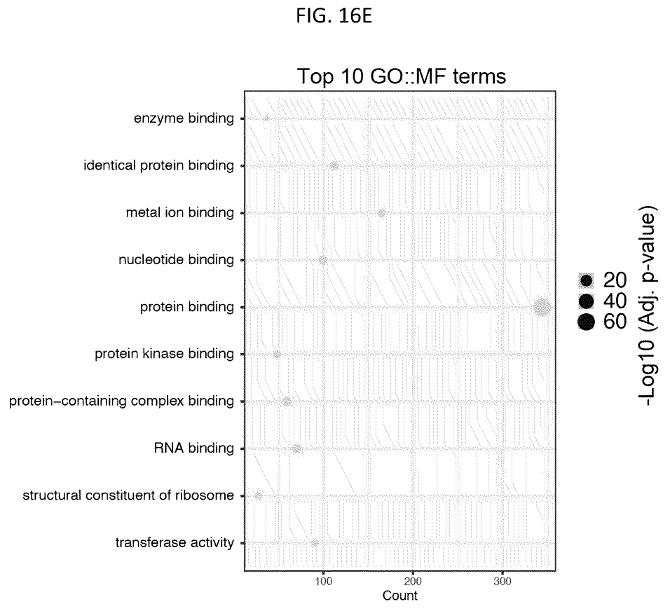

Next, it was investigated whether Gly-EVs can deliver mRNAs to recipient cells (i.e., neurons) and if these mRNAs can be translated within the recipient cells (FIG. 16A). Neurons were treated with a series of Gly-EV doses, and quantitative mass spectrometry was performed (i.e., TMT-MS) was used to quantify the resulting changes in protein expression. Hill dose-response modeling was then applied to assess protein synthesis induced by Gly-EV mRNAs (FIG. 16B). This analysis identified 563 mRNAs from Gly-EVs that led to the synthesis of corresponding proteins (FIG. 16C). Gene annotation analysis revealed that these mRNAs regulate various neuronal functions, including synaptic activity (FIG. 16D, E). These demonstrate that Gly-EVs can deliver mRNAs to recipient neurons, where they are translated into functional proteins. This demonstrates that Gly-EVs can be used as carriers for delivering both protein-based therapeutics (e.g., antibodies) and RNA-based therapeutics (e.g., mRNA, sgRNA, siRNA, shRNA, and ASOs).

What is claimed is:

1. An engineered exosome comprising:
a) a Notch ligand binding domain, wherein the Notch ligand binding domain is expressed on an extracellular surface of the engineered exosome; and
b) a therapeutic cargo,
wherein binding of a Notch ligand to the Notch ligand binding domain induces internalization of the engineered exosome into cells expressing the Notch ligand.

2. The engineered exosome of claim 1, wherein the Notch ligand binding domain comprises a polypeptide having at least 80% sequence identity with SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24.

3. The engineered exosome of claim 3, wherein the Notch ligand binding domain comprises SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24.

4. The engineered exosome of claim 1, wherein the engineered exosome comprises at least a portion of an extracellular domain of a Notch receptor, wherein the extracellular domain includes the Notch ligand binding domain.

5. The engineered exosome of claim 4, wherein the Notch receptor is human Notch1, human Notch2, human Notch3, or human Notch4.

6. The engineered exosome of claim 4, wherein the at least a portion of the extracellular domain of the Notch receptor comprises a polypeptide having at least 80% sequence identity with SEQ ID NO: 5, SEQ ID NO: 6, SEQ ID NO: 7, or SEQ ID NO: 8.

7. The engineered exosome of claim 6, wherein the engineered exosome comprises SEQ ID NO: 5, SEQ ID NO: 6, SEQ ID NO: 7, or SEQ ID NO: 8.

8. The engineered exosome of claim 4, wherein the engineered exosome comprises the extracellular domain and the transmembrane domain of SEQ ID NO: 1, SEQ ID NO: 2, SEQ ID NO: 3, or SEQ ID NO: 4.

9. The engineered exosome of claim 1, wherein the engineered exosome natively expresses a wildtype Notch receptor, such that the Notch ligand binding domain is natively expressed on the extracellular surface of the engineered exosome.

10. The engineered exosome of claim 1, wherein the exosome natively expresses a polypeptide having at least 80% identity with SEQ ID NO: 1, SEQ ID NO: 2, SEQ ID NO: 3, or SEQ ID NO: 4.

11. The engineered exosome of claim 10, wherein the exosome natively expresses the Notch receptor of SEQ ID NO: 1, SEQ ID NO: 2, SEQ ID NO: 3, or SEQ ID NO: 4

12. The engineered exosome of claim 1, wherein the cell is a neuron.

13. The engineered exosome of claim 1, wherein the therapeutic cargo is an agent for the treatment of a neurological disease or condition, a central nervous system cancer, or a central nervous system injury.

14. The engineered exosome of claim 13, wherein the therapeutic cargo is a nucleic acid, a peptide, a protein, an antibody, an aptamer, or a small molecule.

15. The engineered exosome of claim 14, wherein the therapeutic cargo is an mRNA.

16. A method comprising providing to a subject the engineered exosome of claim 1.

17. The method of claim 16, wherein the subject has or is suspected of having a neurological disease or condition, a central nervous system cancer, or a central nervous system injury.

18. A method of treating a neurological disease or condition, a central nervous cancer, or a central nervous system injury in a subject, the method comprising providing to the subject the engineered exosome of claim 13.

19. The method of claim 18, wherein the therapeutic cargo is selectively delivered to neurons in the subject after internalization of the engineered exosome into neurons expressing a Notch ligand.


SEQUENCE LISTING (28 sequences)

SEQ ID NO: 1 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 2 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 3 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 4 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 5 - protein, 1717 aa, Homo sapiens
ARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLH

SEQ ID NO: 6 - protein, 1652 aa, Homo sapiens
LQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQ

SEQ ID NO: 7 - protein, 1603 aa, Homo sapiens
PPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPL

SEQ ID NO: 8 - protein, 1424 aa, Homo sapiens
RGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPW

SEQ ID NO: 9 - protein, 125 aa, Homo sapiens
DVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLG

SEQ ID NO: 10 - protein, 33 aa, Homo sapiens
ANPCEHAGKCINTLGSFECQCLQGYTGPRCEID

SEQ ID NO: 11 - protein, 74 aa, Homo sapiens
VNECVSNPCQNDATCLDQIGEFQCICMPGYEGVHCEVNTDECASSPCLHNGRCLDKINEFQCECPTGFTGHLCQ

SEQ ID NO: 12 - protein, 232 aa, Homo sapiens
DVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQ

SEQ ID NO: 13 - protein, 117 aa, Homo sapiens
DVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTE

SEQ ID NO: 14 - protein, 78 aa, Homo sapiens
DVDECAMANSNPCEHAGKCVNTDGAFHCECLKGYAGPRCEMDINECHSDPCQNDATCLDKIGGFTCLCMPGFKGVHCE

SEQ ID NO: 15 - protein, 38 aa, Homo sapiens
LEINECQSNPCVNNGQCVDKVNRFQCLCPPGFTGPVCQ

SEQ ID NO: 16 - protein, 233 aa, Homo sapiens
DVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQ

SEQ ID NO: 17 - protein, 117 aa, Homo sapiens
NIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCET

SEQ ID NO: 18 - protein, 75 aa, Homo sapiens
DVNECLSGPCRNQATCLDRIGQFTCICMAGFTGTYCEVDIDECQSSPCVNGGVCKDRVNGFSCTCPSGFSGSTCQ

SEQ ID NO: 19 - protein, 38 aa, Homo sapiens
LDVDECASTPCRNGAKCVDQPDGYECRCAEGFEGTLCD

SEQ ID NO: 20 - protein, 230 aa, Homo sapiens
NIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCD

SEQ ID NO: 21 - protein, 118 aa, Homo sapiens
DVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQ

SEQ ID NO: 22 - protein, 80 aa, Homo sapiens
DLDECLMAQQGPSPCEHGGSCLNTPGSFNCLCPPGYTGSRCEADHNECLSQPCHPGSTCLDLLATFHCLCPPGLEGQLCE

SEQ ID NO: 23 - protein, 38 aa, Homo sapiens
VETNECASAPCLNHADCHDLLNGFQCICLPGFSGTRCE

SEQ ID NO: 24 - protein, 236 aa, Homo sapiens
DVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCE

SEQ ID NO: 25 - DNA, 35 bp, Synthetic construct
  source 1..35: mol_type = other DNA
acttgcaacaagcaattgccagagaacctagtgct

SEQ ID NO: 26 - DNA, 35 bp, Synthetic construct
  source 1..35: mol_type = other DNA
gttctctggcaattgcttgttgcaagtcctttaag

SEQ ID NO: 27 - DNA, 35 bp, Synthetic construct
  source 1..35: mol_type = other DNA
acttgcaacaagatattgccagagaacctagtgct

SEQ ID NO: 28 - DNA, 35 bp, Synthetic construct
  source 1..35: mol_type = other DNA
gttctctggcaatatcttgttgcaagtcctttaag